(19) United States
(12) Patent Application Publication
(10) Pub. No.: US 2015/0183737 A1
(43) Pub. Date: Jul. 2, 2015

(54) COMPOSITIONS AND METHODS FOR SUBSTRATE-SELECTIVE INHIBITION OF ENDOCANNABINOID OXYGENATION

(71) Applicant: Vanderbilt University, Nashville, TN (US)

(72) Inventors: Lawrence J. Marnett, Nashville, TN (US); Sachin Patel, Nashville, TN (US); Daniel Hermanson, Cincinnati, OH (US); Matthew A. Windsor, Nashville, TN (US)

(21) Appl. No.: 14/415,977

(22) PCT Filed: Jul. 22, 2013
(86) PCT No.: PCT/US2013/051523

§ 371 Date: Jan. 20, 2015

Related U.S. Application Data
(60) Provisional application No. 61/673,807, filed on Jul. 20, 2012.

(51) Int. Cl. C07D 209/14; C07C 233/27; C07C 62/38; C07C 57/62; C07C 59/72; C07C 215/76; C07C 233/25

(52) U.S. Cl. CPC C07D 209/14; C07C 215/76; C07C 233/27; C07C 233/25; C07C 57/62; C07C 59/72; C07C 62/38

(57) ABSTRACT

Methods for selectively inhibiting endocannabinoid oxygenation but not arachidonic acid oxygenation. In some embodiments, the methods include contacting a COX-2 polypeptide with an effective amount of a substrate-selective COX-2 inhibitor. Also provided are methods for elevating a local endogenous cannabinoid concentrations; methods of reducing depletion of an endogenous cannabinoid; methods for inducing analgesia; methods of providing anxiolytic therapy; methods for providing anti-depressant therapy; and compositions for performing the disclosed methods.

### CROSS REFERENCE TO RELATED APPLICATION

The presently disclosed subject matter claims the benefit of U.S. Provisional Patent Application Ser. No. 61/673,807, filed Jul. 20, 2012; the disclosure of which is incorporated herein by reference in its entirety.

### GOVERNMENT INTEREST

This invention was made with government support under Grant Nos. CA89450, DA02014, DA022873, DA022873, DA031572, ES07028, GM15431, HL109199, HL96967, MH063232, MH090412, MH100096, NS064278, NS078291, R13DA016280, T32-MH065215, 5P41RR015301-10, and 8P41GM103403-10 awarded by the National Institutes of Health of the United States. The Government has certain rights in the invention.

### TECHNICAL FIELD

The presently disclosed subject matter relates to compositions for inhibiting a biological activity of a cyclooxygenase (COX) polypeptide. In some embodiments, the compositions comprise derivatives of non-steroidal anti-inflammatory drugs (NSAIDs) that are COX-1-specific, COX-2-specific, or are non-specific COX inhibitors. In some embodiments, the methods comprise administering a composition comprising an inhibitor of the presently disclosed subject matter to a subject in order to modulate a COX-2 biological activity. In some embodiments, the methods comprise administering a composition comprising an inhibitor of the presently disclosed subject matter to a subject in order to modulate a subset of COX-2 biological activities including, but not limited to endocannabinoid oxygenation catalyzed by COX-2. In some embodiments, the modulation of the subset of COX-2 biological activities does not substantially affect other COX-2 biological activities including, but not limited to arachidonic acid oxygenation.

### BACKGROUND

Non-steroidal anti-inflammatory drugs (NSAIDs) are a class of therapeutic agents that are widely used for their anti-inflammatory and anti-pyretic properties to treat human distress and disease. Exemplary NSAIDs include aspirin, ibuprofen, acetaminophen, indomethacin, naproxen, and others.

The anti-inflammatory and anti-pyretic activities of NSAIDs derive from the ability of these compounds to bind to and inhibit the actions of the cyclooxygenase (COX) enzymes. COX activity originates from two distinct and independently regulated enzymes, termed cyclooxygenase-1 (COX-1) and cyclooxygenase-2 (COX-2; see DeWitt & Smith, 1988; Yokoyama & Tanabe, 1989; Hla & Neilson, 1992). COX-1 is a constitutive isoform and is mainly responsible for the synthesis of cytoprotective prostaglandins in the gastrointestinal (GI) tract and for the synthesis of thromboxane, which triggers aggregation of blood platelets (Allison et al., 1992). On the other hand, COX-2 is inducible and short-lived. Its expression is stimulated in response to endotoxins, cytokines, and mitogens (Kujubu et al., 1991; Lee et al., 1992; O'Sullivan et al., 1993). NSAIDs exhibit varying selectivity for COX-1 and COX-2 but, in general, most display inhibitory activity towards both enzymes (Meade et al., 1993).

The COX enzymes are homodimers of 70 kiloDalton (kDa) subunits that are comprised of membrane-binding and catalytic domains (Garavito et al., 2003). The cyclooxygenase active site is located deep inside the catalytic domain separated by a gate from a channel that leads through the membrane-binding domain to the exterior of the protein. The two monomers of each COX enzyme are functionally interdependent and binding of a substrate or inhibitor at one active site alters the properties of the other active site (Yuan et al., 2006). The communication between subunits occurs through the dimer interface (Yuan et al., 2009).

COX enzymes oxygenate polyunsaturated fatty acids to prostaglandin endoperoxides in the first step of a metabolic cascade that leads to the generation of prostaglandins and thromboxanes. Inhibition of COX enzymes, especially COX-2, is a major contributor to the pharmacological effects of NSAIDs. COX-2 oxygenates a range of fatty acyl substrates including fatty acids, esters, and amides. Arachidonic acid (AA) and 2-arachidonoylglycerol (2-AG) are the best acid and ester substrates and display comparable k_{cat}/Kₘ's for oxygenation (Kozak et al., 2000).

2-AG and arachidonoylethanolamide (AEA) are endocannabinoids that exert anxiolytic, analgesic, and anti-inflammatory effects through their actions at the cannabinoid receptors, CB1 and CB2 (Piomelli, 2003; Di Marzo et al., 2005; Kogan & Mechoulam, 2006). They are also substrates for the fatty acid oxygenases, lipoxygenases, and cyclooxygenases (COXs), as well as for certain cytochromes of the P450 family, which convert them to bioactive, oxygenated metabolites (Ueda et al., 1995; Yu et al., 1997; Kozak et al., 2000; Chen et al., 2008; Snider et al., 2010). 2-AG and AEA are oxygenated efficiently to prostaglandin glycerol esters (PG-Gs) and prostaglandin ethanolamides (PG-EAs), respectively, by COX-2, but much less so than by COX-1. PG-Gs and PG-EAs activate calcium mobilization in macrophages and tumor cells, enhance miniature excitatory and inhibitory postsynaptic currents in neurons, induce mechanical allodynia, and stimulate thermal hyperalgesia (Nirodi et al., 2004; Sang et al., 2006; Snag et al., 2007; Hu et al., 2008; Richie-Jannetta et al., 2010). 2-AG and AEA are rapidly hydrolyzed by monoacylglycerol lipase (MAGL) and fatty acid amide hydrolase (FAAH), respectively, to arachidonic acid (AA), which terminates endocannabinoid signaling but produces a fatty acid that is converted to leukotrienes and prostaglandins, among others (Cravatt et al., 1996; Dinh et al., 2004). Thus, 2-AG and AEA are at the nexus of a complex network of bioactive lipid production, inactivation, and signaling (see FIG. 1).

The importance of endocannabinoids as naturally occurring analgesic agents provides a potential mechanism for inhibition of neuropathic pain: that is, through the development of agents that prevent endocannabinoid metabolism at sites of neuroinflammation (Piomelli et al., 2000). FAAH inhibitors seem to be promising candidates in this regard, and MAGL inhibitors are also potential leads, although their broader range of cannabimimetic effects in animal models potentially limit their utility (Cravatt & Lichtman, 2003; Long et al., 2009a). What is needed, then, are additional methods and compositions for inhibiting a biological activity of a cyclooxygenase (COX) polypeptide, such as but not limited to a subset of COX-2 biological activitites including, but not limited to endocannabinoid oxygenation catalyzed by COX-2. Such methods and compositions represent a long-felt and continuing need in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides in some embodiments methods for selectively inhibiting endocannabinoid oxygenation but not arachidonic acid oxygenation. The presently disclosed subject matter also provides in some embodiments methods for elevating a local endogenous cannabinoid concentration in a tissue, cell, organ, and/or structure in a subject.

The presently disclosed subject matter also provides in some embodiments methods for reducing depletion of an endogenous cannabinoid in a tissue, cell, organ, and/or structure in a subject.

The presently disclosed subject matter also provides in some embodiments methods for inducing analgesia in a subject.

The presently disclosed subject matter also provides in some embodiments methods for providing an anxiolytic and antidepressant therapy to a subject. In some embodiments, the presently disclosed methods comprise contacting a COX-2 polypeptide with an effective amount of a substrate-selective COX-2 inhibitor (referred to herein as an “SSCI”).

In some embodiments of the presently disclosed methods, the substrate-selective inhibitor of COX-2 (SSCI) comprises a substantially pure (R)-profen or a derivative thereof, optionally wherein the derivative thereof does not stereoisomerize in vivo to an (S)-profen. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

In some embodiments of the presently disclosed subject matter, the COX-2 polypeptide is present in the tissue, cell, organ, and/or structure in the subject and/or is present in a distant location in the subject that under normal conditions provides an endogenous cannabinoid to the tissue, cell, organ, and/or structure in the subject. In some embodiments, the COX-2 polypeptide is present in a region of inflammation in the subject.

The presently disclosed subject matter also provides compounds. In some embodiments, the compound is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, and an (R)-enantiomer of Compound 6e.

In some embodiments, the presently disclosed subject matter provides pharmaceutical compositions comprising, consisting essentially of, or consisting of a substrate-selective inhibitor of COX-2 and a pharmaceutically acceptable carrier or excipient, optionally wherein the pharmaceutically acceptable carrier or excipient is acceptable for use in a human. In some embodiments, the substrate-selective inhibitor of COX-2 comprises a substantially pure (R)-profen or a derivative thereof, optionally wherein the derivative thereof does not stereoisomerize in vivo to an (S)-profen. In some embodiments, the (R)-profen or a derivative thereof is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, Compounds acetaminophen (APAP), 4-aminophenol, and N-(4-hydroxyphenyl)arachidonoylamide (AM404), and combinations thereof.

Thus, it is an object of the presently disclosed subject matter to provide compositions and methods to selectively inhibit endocannabinoid oxygenation but not arachidonic acid oxygenation by cyclooxygenases. An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE DRAWINGS

FIG. 1 is a schematic diagram of the endocannabinoid metabolism. The pathways for the release of 2-AG, AA, and AEA, their oxygenation by COX-2, and the hydrolysis of 2-AG by MAGL and of AEA by FAAH are illustrated.

FIG. 2 is a schematic diagram showing oxygenation catalyzed by COX-2. Substrates and products are shown underneath.

FIG. 3 is a schematic diagram of an exemplary synthesis scheme for producing achiral Profens. In the Scheme, the reagents and conditions are as follows: (i) Ph₃P═CHOMe, t-BuOK, THF, 0° C., 45 min, rt, 1 hr. (ii) 5:2 THF:5 N HCl, reflux, 1 hr. (iii) 2,3-methylbutene, KH₂PO₄, NaClO₂, 1:1 t-BuOH:H₂O, 40 min, rt. (iv) H₂SO₄, MeOH, reflux, 2 hr. (v) LDA, THF, 30 min, −78° C., HMPA, 30 min, 0° C., 1,2-dibromoethane, 30 min, rt. (vi) KOTMS, THF, reflux, 2 hr. (vii) LDA, THF, 30 min, −78° C., HMPA, 30 min, 0° C., iodomethane, 30 min, rt. (viii) CHIRALCEL® AD column, 90:10 hexane:IPA, 0.1% TFA.

FIGS. 4B and 4C depict analysis of dorsal root ganglion cells (DRGs).

FIG. 4A depicts western blot analysis of basal versus stimulated DRGs comparing enzymes involved in endocannabinoid metabolism and prostaglandin synthesis.

FIG. 4B is a series of LC-MS chromatographic peaks and selected reaction monitoring transitions for prostaglandins derived from arachidonic acid, AEA, and 2-AG isolated from DRGs

FIG. 5 is a series of graphs summarizing inhibition studies of eicosanoid synthesis in stimulated DRGs by (R)-flurbiprofen, (R)-naproxen and (R)-ibuprofen.

Product formation was monitored following the oxygenation of arachidonic acid, 2-AG and AEA by COX-2 to form prostaglandins (solid lines), PG-Gs (dashed lines), and PG-EAs (dotted lines) in DRGs. IC₅₀ values were calculated using a nonlinear regression. The data points represent percent inhibition with respect to a control consisting of two sets of three DRG culture plates from two independent DRG preparations for each (R)-profen. The data points (n=6) represent the mean+s.e.m.

FIG. 6 is a graph showing the time-dependence of inhibition of COX-2-mediated 2-AG metabolism by (R)-profens. Filled circles: 4 μzM (R)-flurbiprofen; open circles: 2-μM (R)-flurbiprofen; triangles: 20 μM Naproxen.

FIG. 7 is a graph summarizing a comparison of the concentration dependence of (R)-flurbiprofen inhibition of 2-AG oxygenation between wild type (diamonds) and mutant variants of mCOX-2 (R120Q in circles, Y335F in squares, E524L in triangles, and S530A in upside down triangles). Inhibition assays were performed as described herein below in the Materials and Methods section of the EXAMPLES. The data points (n=3) represent the mean±s.e.m.

FIG. 8 is a series of Western blot analyses of basal (left lane) versus stimulated (right lane) DRG assays.

FIG. 9 is a comparison of MS/MS fragmentation patterns of the compounds eluting at the positions of PGF_{2α}-EA and PGE-EA isolated from DRGs to standards.

FIG. 10 presents a series of bar graphs summarizing comparisons of the effects of (R)-flurbiprofen, (R)-naproxen, and (R)-ibuprofen on substrate concentrations in basal versus stimulated DRGs. The data points represent the amount of AEA (first bar of each set of three), 2-AG (second bar of each set of three), and arachidonic acid (third bar of each set of three) from two sets of three DRG culture plates from two independent DRG preparations for each (R)-profen.

The fatty acid concentrations (n=6) are plotted as mean+s.e.m. and statistical significance was determined using a one-way ANOVA analysis. Statistically significant increases (P<0.05) in both AEA and 2-AG are indicated by overhead brackets.

FIG. 11 is a series of plots showing inhibition of human MAGL in vitro by (R)-flurbiprofen, (R)-naproxen, (R)-ibuprofen, and the known MAGL Inhibitor JZL 184. Each compound was pre-incubated with human recombinant MAGL for 5 minutes at 37° C. before addition of 50 μM 2-AG for 5 minutes. The reactions were quenched and conversion of 2-AG to AA was quantified using SRM LC-MS-MS.

Silver-associated fatty acid ions were monitored using the following transitions: 2-AG 485→411, 2-AG-d8 493→419, AA 519→409, AA-d8 527→417. Minimal inhibition of MAGL by (R)-profens was observed while the IC₅₀ for JZL-184 was 9 nM.

FIG. 12 is a series of plots showing inhibition of humanized rat FAAH by (R)-profens in vitro. The inhibition of FAAH by (R)-flurbiprofen, (R)-naproxen, and (R)-ibuprofen was measured and compared to FAAH inhibition by URB 597, a known FAAH inhibitor. The inhibitors were pre-incubated with humanized rat FAAH for 5 minutes at 37° C. prior to the addition of 50 μM AEA. The reaction was quenched after 5 minutes, and conversion of AEA to AA was quantified by SRM LC-MS/MS. The levels of AEA and AA were monitored using the following transitions: AEA 456→438, AEA-d8 464→446, AA 519→409, and AAd8 527→417 for silver-associated ions. The IC₅₀ value for URB597 was determined to be 2 nM but no inhibition of FAAH by (R)-profens was observed at concentrations up to 1 mM.

FIG. 13 is a series of plots showing inhibition of human 15-lipoxygenase-1 by (R)-flurbiprofen, (R)-naproxen, and (R)-ibuprofen in vitro. The extent of inhibition was assessed by pre-incubating the inhibitors with enzyme for 5 minutes at 37° C. followed by the addition of 50 μM of either AA or 2-AG for 30 seconds. The reactions were then quenched and conversion of AA or 2-AG to 15-HETE (i) or 15-HETE-G (o) was detected and quantified using HPLC and UV absorbance at 236 nm. The 15-HETE and 15-HETE-G were distinguished based on retention time and a standard curve was used to quantify the peak areas.

FIG. 14 is a series of plots showing analysis of (R)-flurbiprofen-, (R)-naproxen-, and (R)-ibuprofen-treated DRG extracts using chiral HPLC and fluorescence. A Daicel chiralpak chiral column was used in conjunction with a normal-phase HPLC gradient to separate the profen enantiomers. Conversion of (R)-flurbiprofen, (R)-naproxen, and (R)-ibuprofen to (S)-flurbiprofen, (S)-naproxen, and (S)-ibuprofen did not occur over the time course employed in the experiments summarized in the Figure. An analysis of standard mixtures of flurbiprofen, naproxen, and ibuprofen enantiomers showed the chromatography and separation of the two enantiomers.

FIG. 15 depicts a proposed mechanism of COX-2 substrate-selective inhibition of endocannabinoid oxygenation by rapid, reversible inhibitors. Inhibitor binding in one subunit of the homodimer induces a conformational change in the second subunit that blocks 2-AG and AEA oxygenation but not arachidonic acid oxygenation. To inhibit oxygenation of arachidonic acid, another molecule of inhibitor must bind in the second subunit. For slow, tight-binding inhibitors, the conformational changes induced by binding a single inhibitor molecule are sufficient to inhibit the oxygenation of all substrates. I, inhibitor; AA, arachidonic acid; PG, prostaglandin.

FIG. 16 depicts a structure of Compound A, an exemplary substrate-selective inhibitor of the presently disclosed subject matter. FIG. 17 is a schematic diagram of α-substituents of desmethyl (Compounds 3a-e), dimethyl (Compounds 4a-e), cyclopropyl (Compounds 5a-e), racemic (Compounds 6a-e) and (R)-(Compounds 7a-e) profens. Aryl scaffolds of flurbiprofen (a), naproxen (b), ibuprofen (c), fenoprofen (d), and ketoprofen (e) are also depicted. FIG. 18 is two plots of spectra of Compound 7d (top) and Compound 7e (bottom) at 260 nm.

FIGS. 19A-19D are a series of plots showing oxygenation of 2-AG and AA vs. inhibitor concentration in RAW 264.7 cells. The dotted lines describe the percent conversion of AA to PGE2/PGD2 and the solid lines describe the percent conversion of 2-AG to PGE2-G/PGD2-G. FIG. 19A) shows the results with Compound 3a. 2-AG IC₅₀=0.6 μM, 60% AA inhibition at 5 μM Compound 3a.

FIG. 19B shows the results with Compound 4a. 2-AG IC₅₀=5.2 μM, 40% AA inhibition at 25 μM Compound 4a. FIG. 19C shows the results with Compound 5a. 2-AG IC₅₀=10.2 μM, 55% AA inhibition at 25 μM Compound 5a. FIG. 19D shows the results with Compound 7a. 2-AG IC₅₀=1.3 μM, 0% AA inhibition at 50 μM Compound 7a.

FIGS. 20A-20J depict molecular determinants of substrate-selective pharmacology. FIG. 20A is a graph showing indomethacin inhibition of AA (squares), 2-AG (circles), and AEA (triangles) oxygenation by WT mCOX-2. FIG. 20B is a graph showing indomethacin inhibition of 2-AG (circles) but not AA (squares) oxygenation by R120Q COX-2. FIG. 20C is a graph showing indomethacin inhibition of 2-AG (circles) but not AA (squares) oxygenation by Y355F COX-2. FIG. 20D is a schematic depiction of an exemplary reaction that can be used for the conversion of indomethacin to Compound A, an SSIC. FIG. 20E is a graph showing Compound A inhibition of AEA (triangles) and 2-AG (circles), but not AA (squares), oxygenation by wild type mCOX-2. FIG. 20F is a graph showing inhibition of 2-AG (circles), but not AA (squares), oxygenation by COX-2 in stimulated RAW 264.7 macrophages by Compound A. FIG. 20G is a plot showing levels of 2-AG (first bar of each pair) and AA (second bar of each pair) in stimulated RAW 264.7 macrophages in response to increasing concentrations of Compound A. Compound A significantly increased 2-AG levels at 1.5 μM (p=0.024) and 3 μM (p=0.007). Data shown are mean±S.E.M with n=3 for each point. Significance was determined using a one-way ANOVA followed by Holm-Sidak's multiple comparisons post-test. FIG. 20H is a graph showing the effects of Compound A (circles), PF-3845 (triangles), and URB597 (squares) on FAAH activity. FIG. 20I is a graph showing the effects of Compound A (top plot) and JZL-184 (lower plot) on MAGL activity. FIG. 20J is a graph showing the effects of Compound A (circles) and THL (squares) on DAGL activity.

FIGS. 21a-21n are a series of graphs demonstrating that Compound A (also referred to herein as “LM-4131”) is an in vivo bioactive substrate-selective COX-2 inhibitor (SSCI). FIGS. 21a-21d are a series of bar graphs show the effects of increasing doses of Compound A on AEA, 2-AG, AA and PG, respectively, in brain 2 hours after i.p. injection. FIGS. 21e and 21f are plots of combined data from multiple cohorts of mice showing the average magnitude of Compound A effects on brain AEA and 2-AG levels as % vehicle treatment. FIGS. 21g-21j are a series of bar graphs showing the effects of Compound A, indomethancin, NS-398, and SC-560 on brain. FIG. 21g: AEA; FIG. 21h: 2-AG; FIG. 21i: AA; and FIG. 21j:

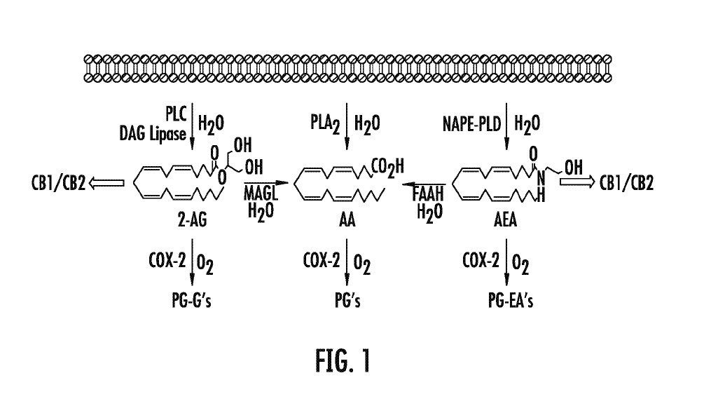

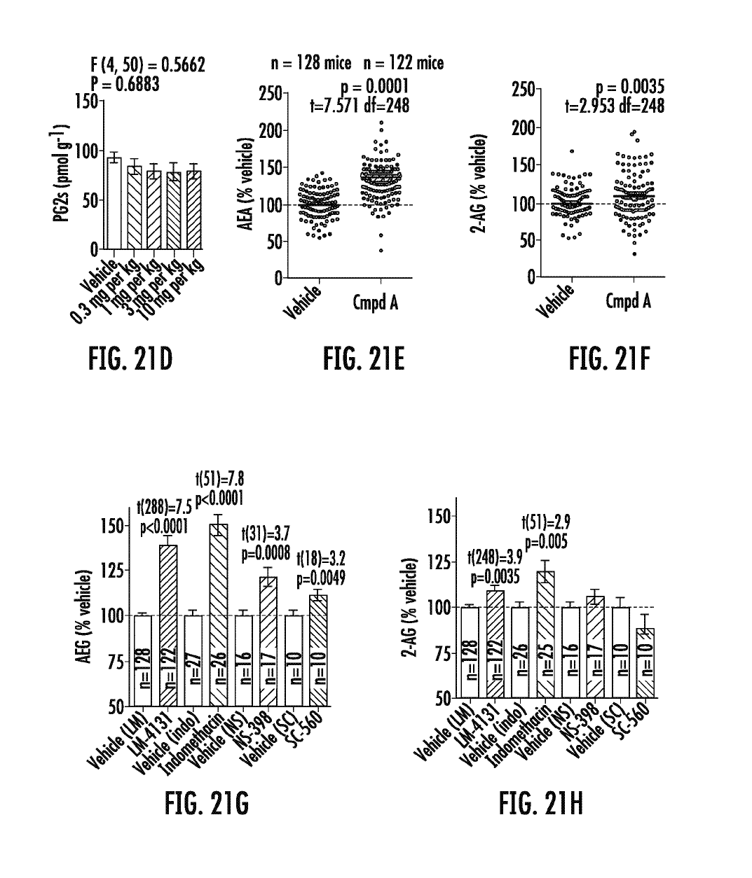

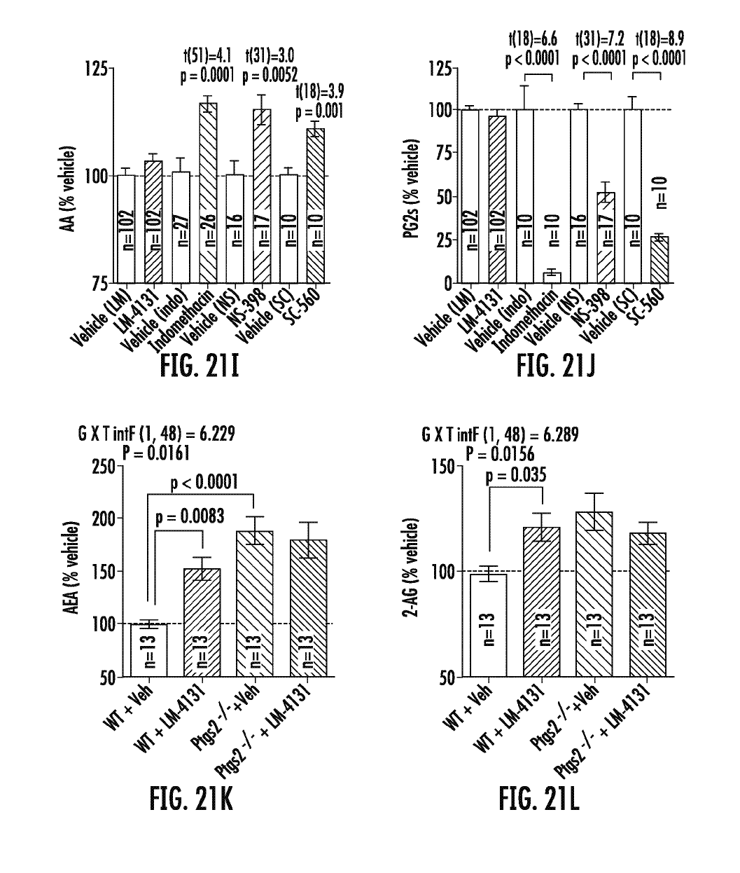

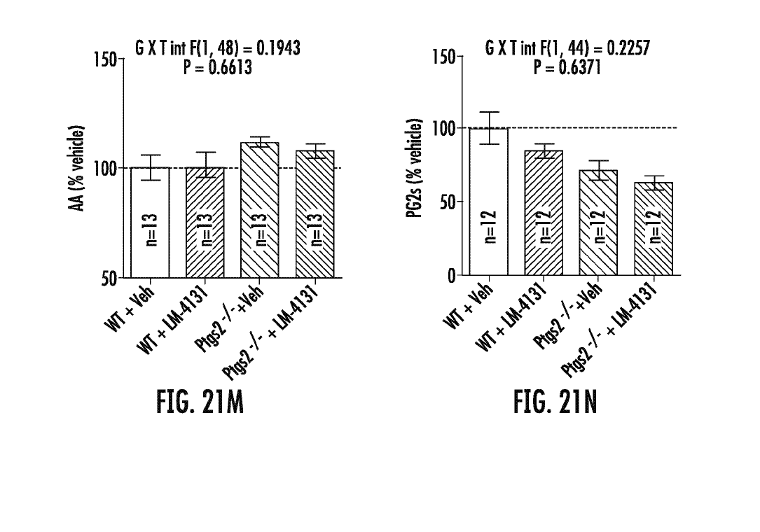

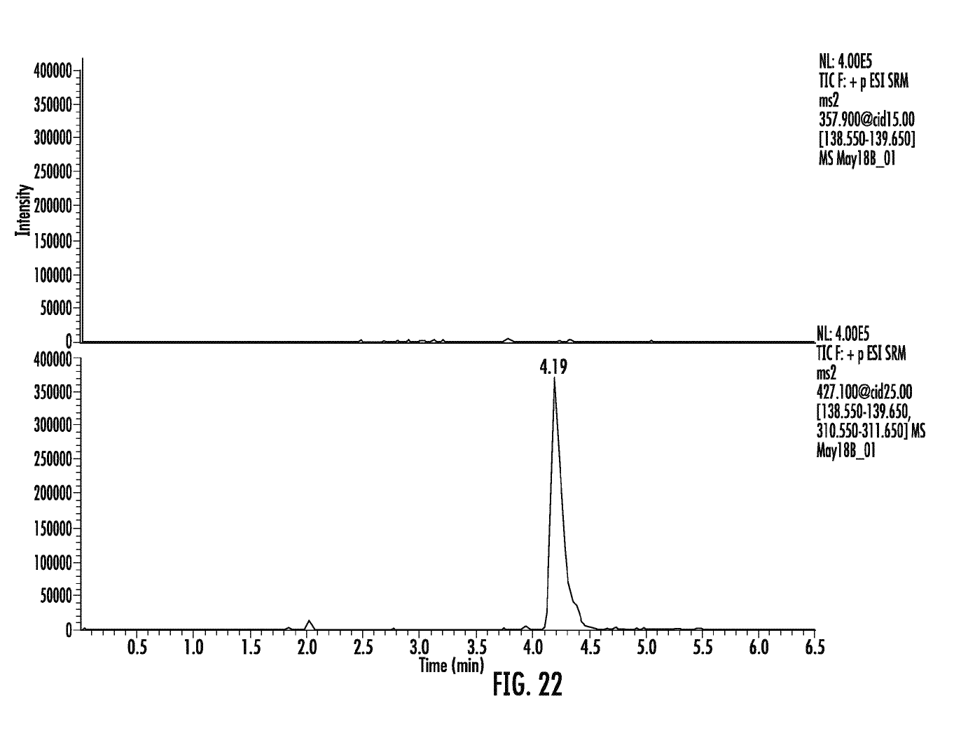

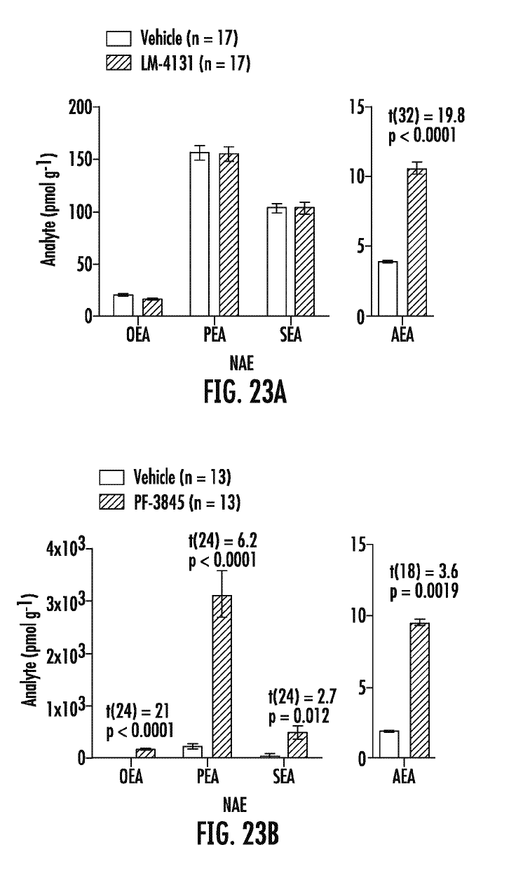

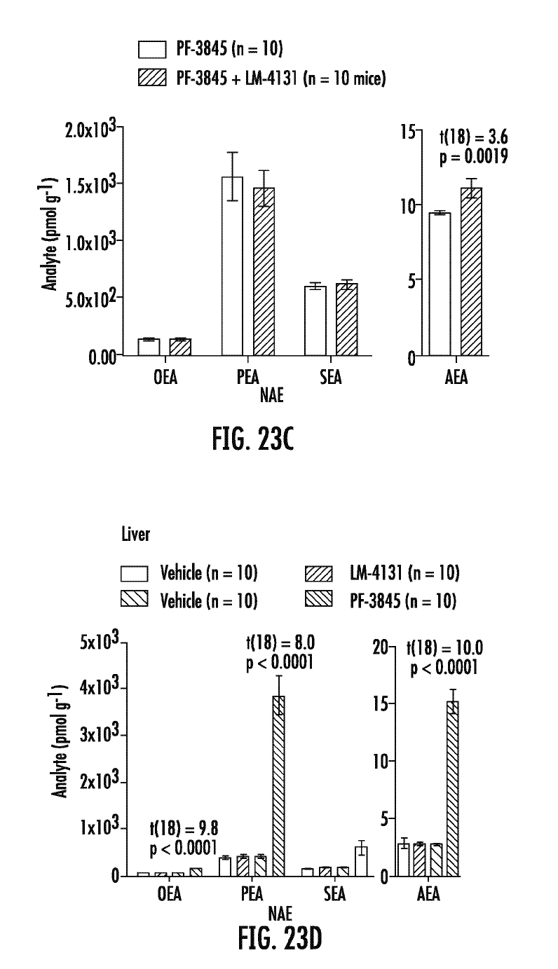

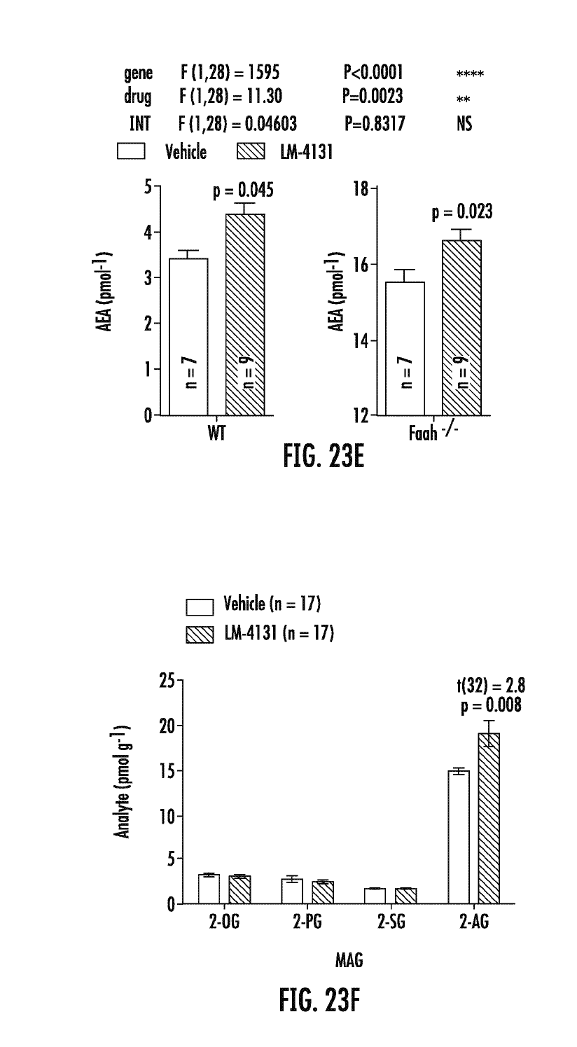

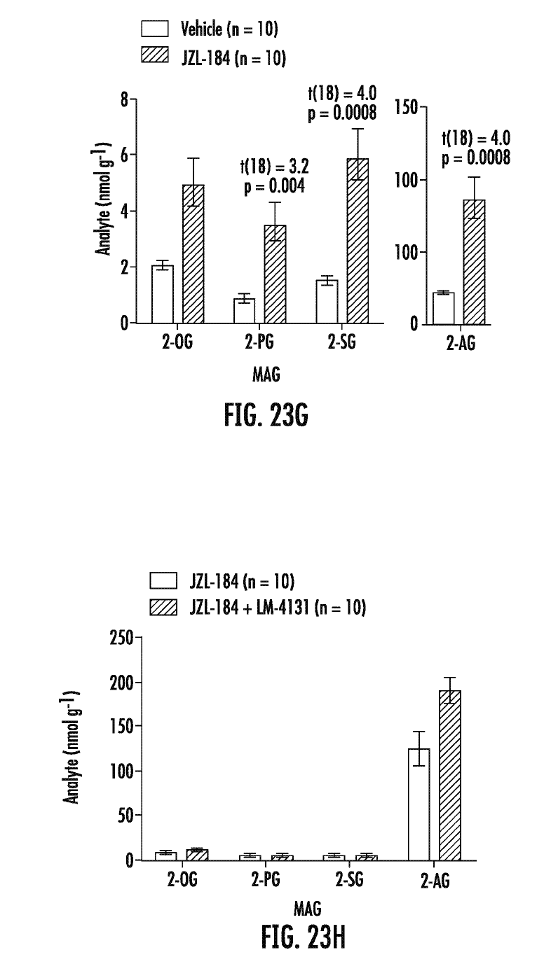

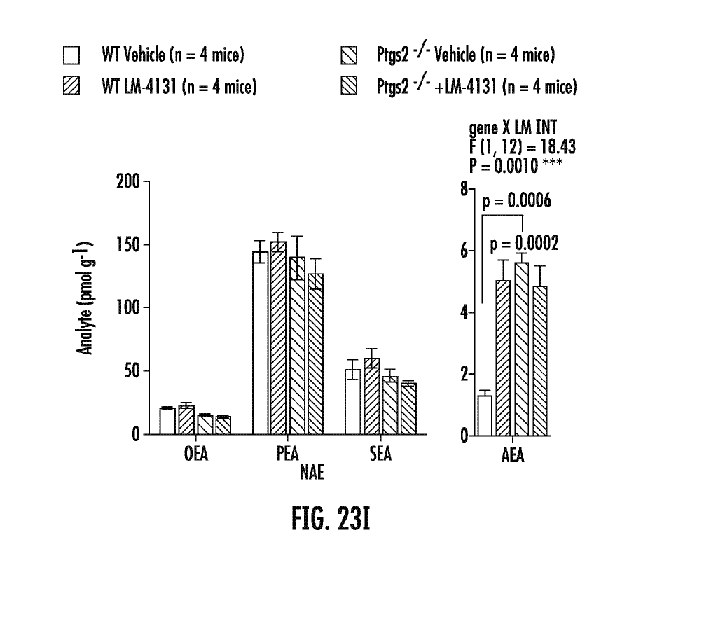

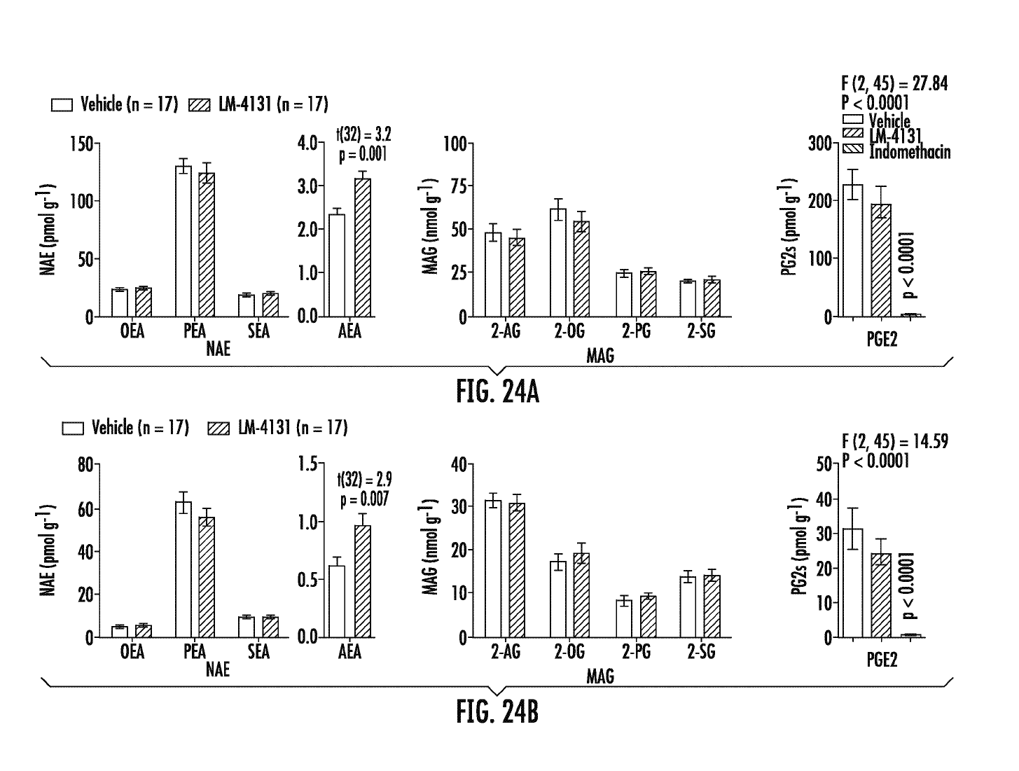

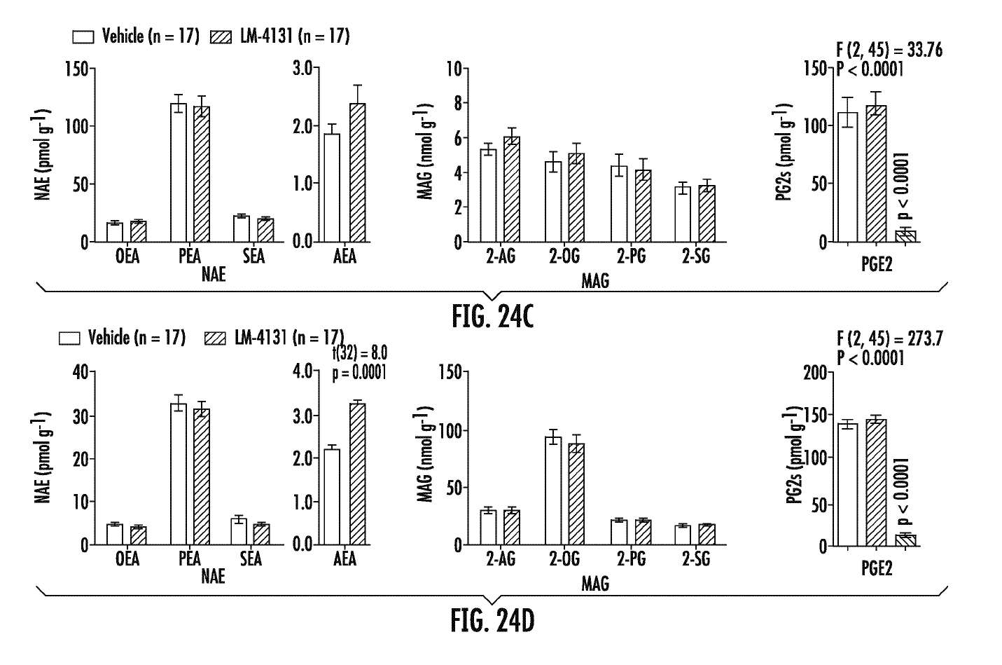

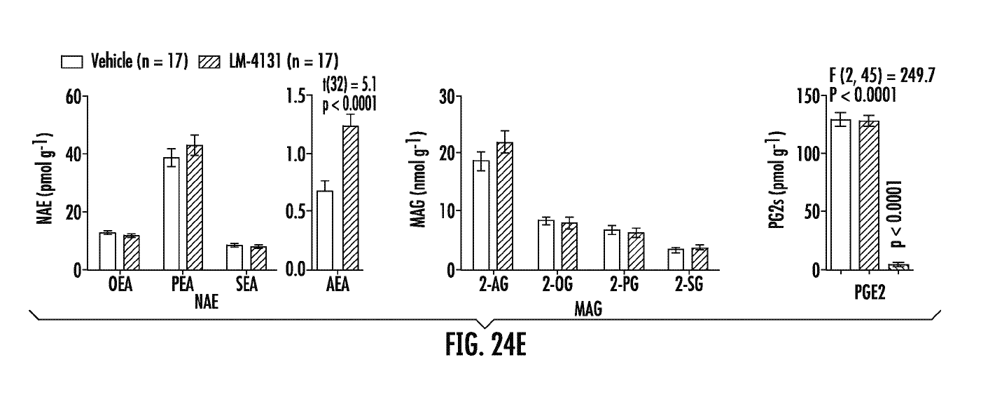

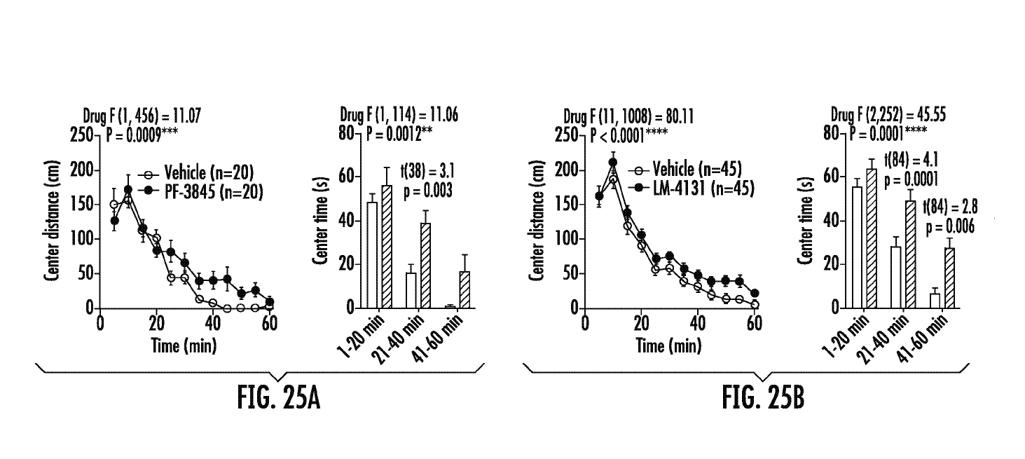

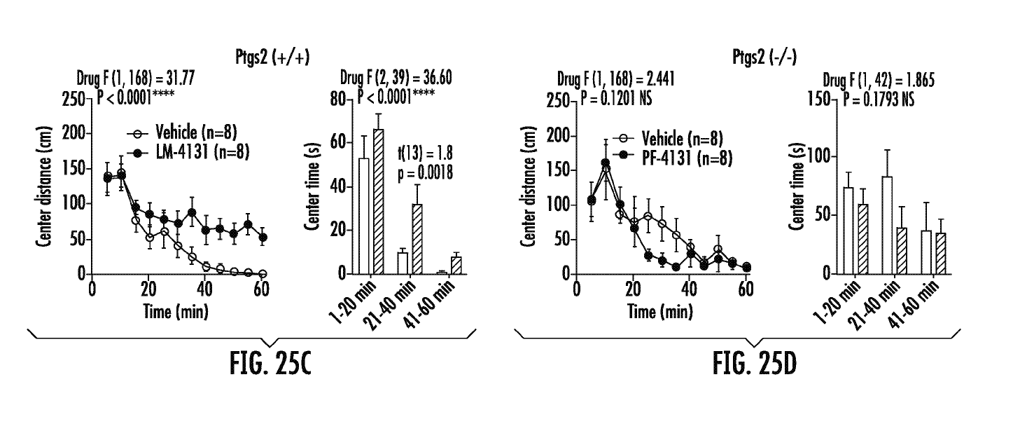

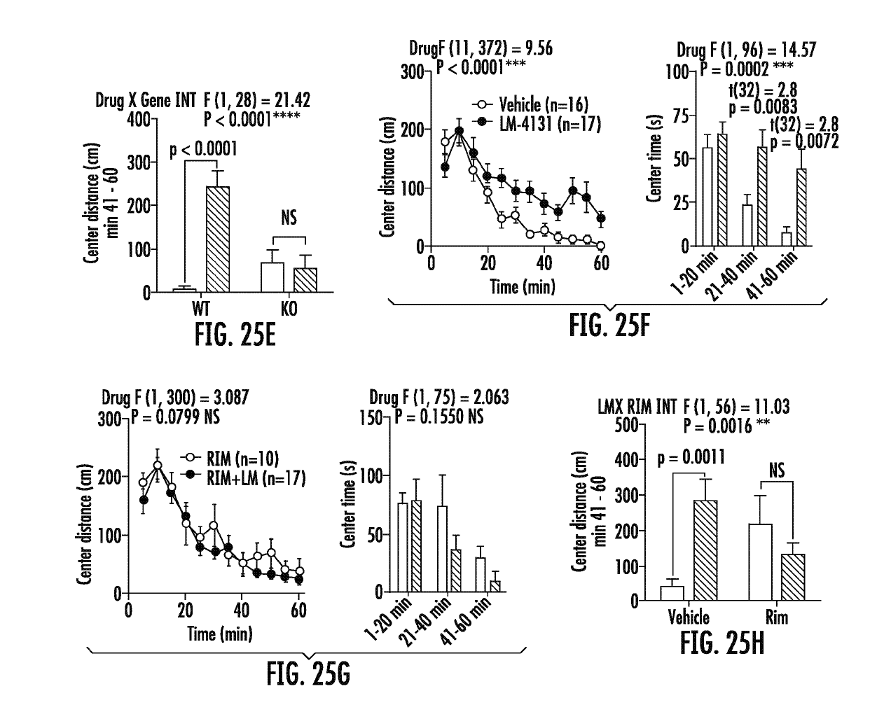

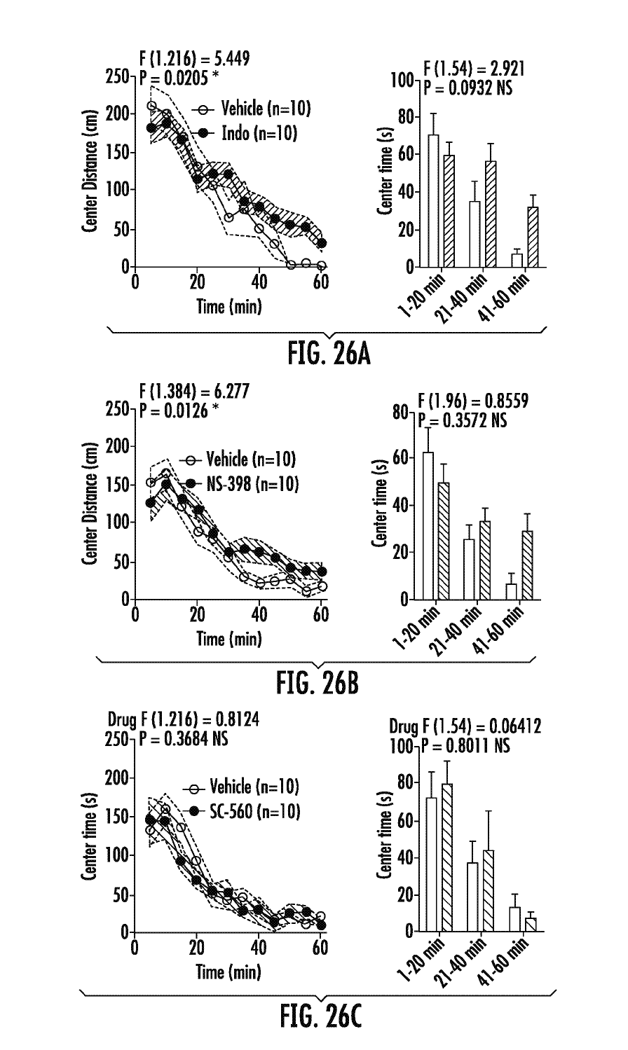

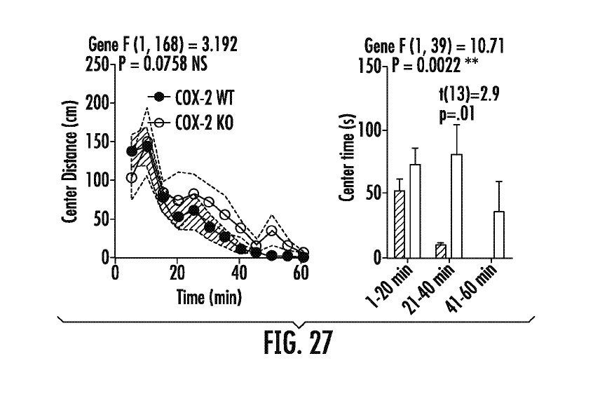

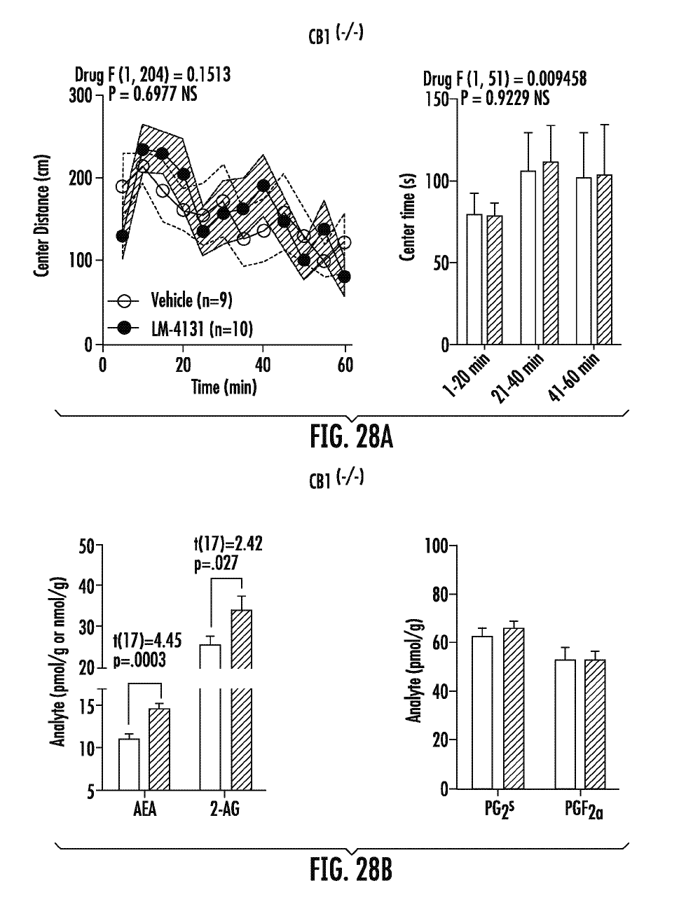

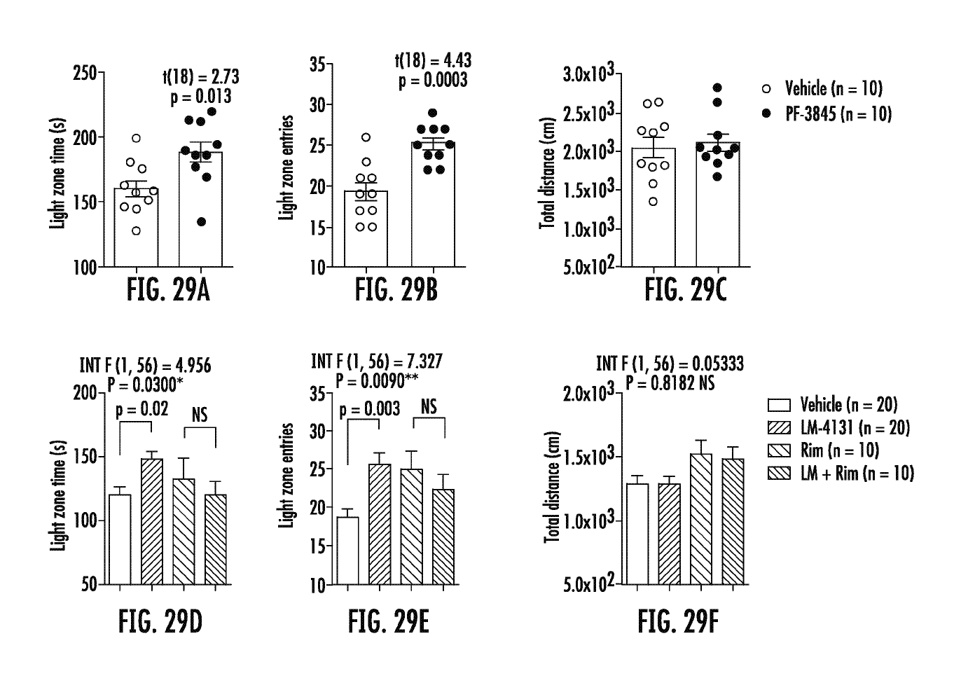

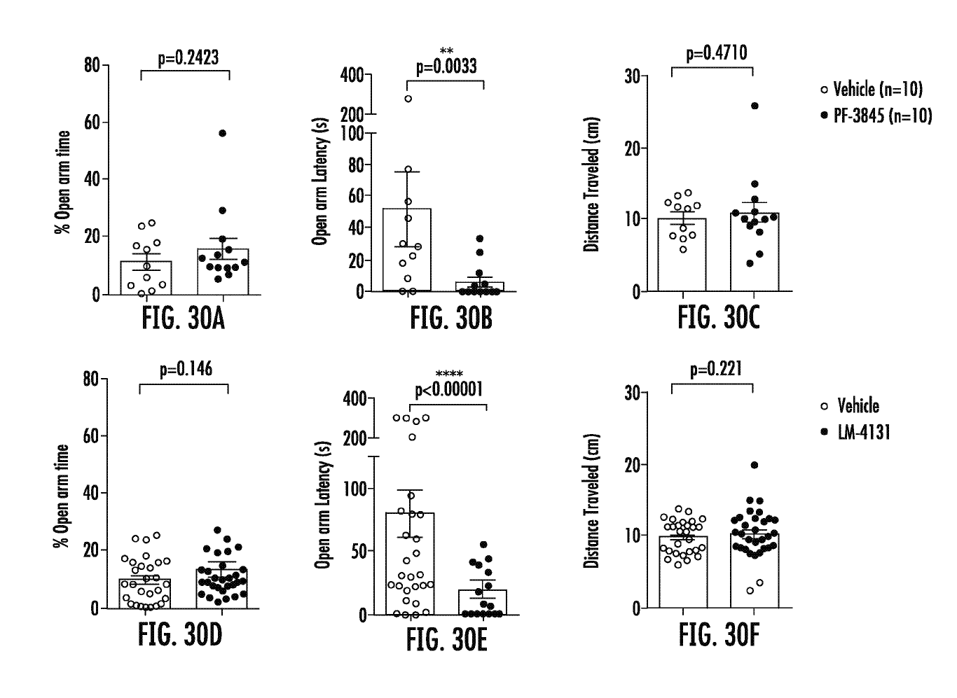

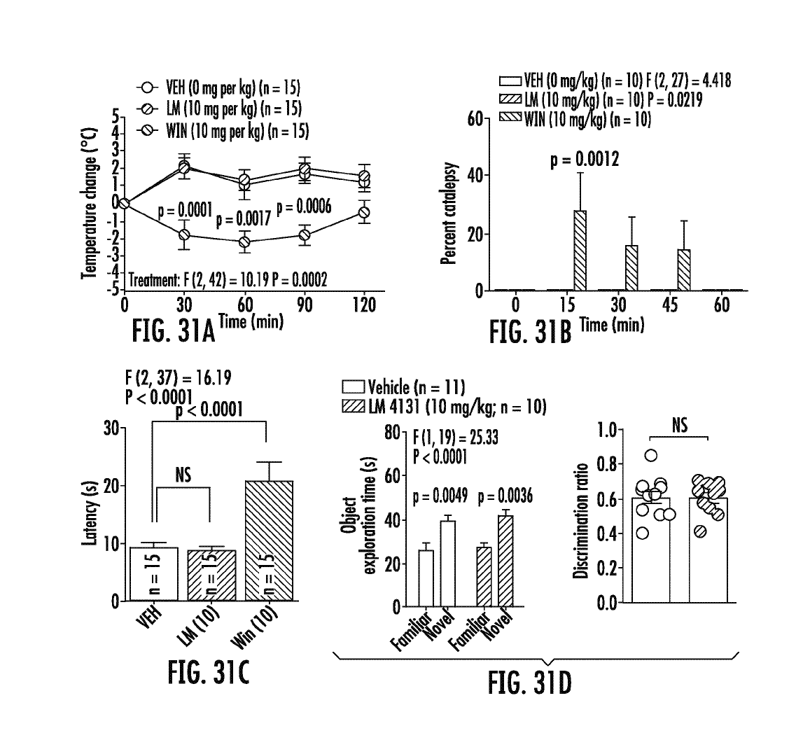

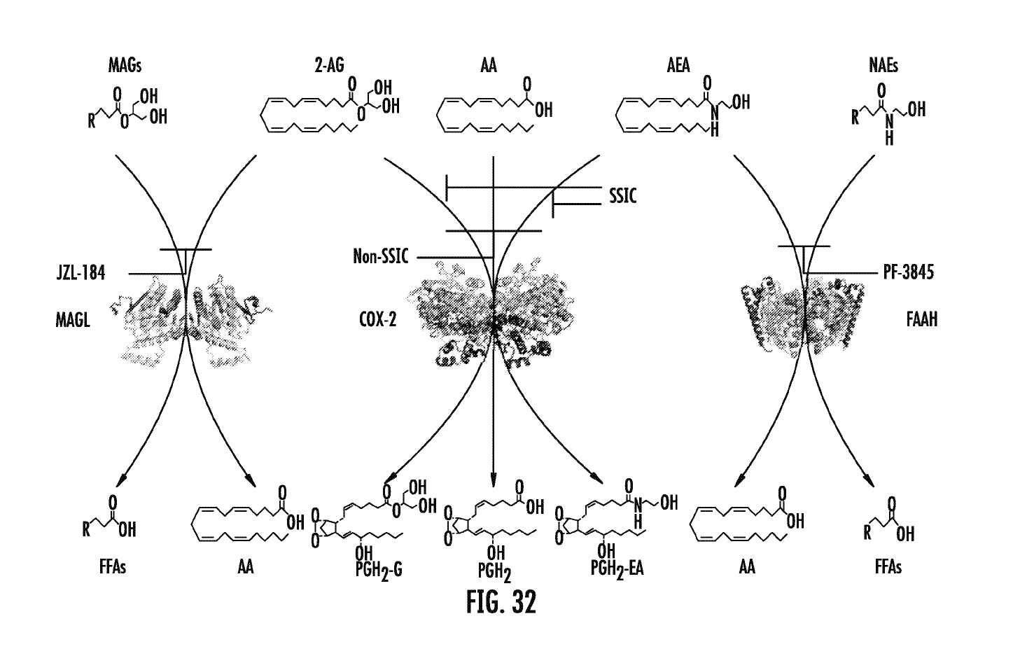

PG levels as a % of corresponding vehicle group. FIGS. 21k-21n are a series of bar graphs showing the effects of Compound A on brain AEA (FIG. 21k); 2-AG (FIG. 21l); AA (FIG. 21m); and PG (FIG. 21n) in WT and COX-2 KO mice.

F and p values shown for one-way ANOVA, and p values for Dunnett's post hoc analysis show in FIGS. 21a-21d; t-statistics and p values shown for unpaired two-tailed t-tests in FIGS. 21e and 21f and FIGS. 21g-21j; drug treatment vs. corresponding vehicle group); F and p values for genotype X LM-treatment interaction (G×T int) by two-way ANOVA, and p values for Sidak's multiple comparisons post hoc test shown in FIGS. 21k and 21l. n=number of mice per treatment group indicated in bars. Error bars represent S.E.M. FIG. 22 is a plot showing a representative detection of Compound A in brain extract. Compound A (bottom plot) was detected in brain 2 hours after i.p. injection using SRM LC-MS/MS with a parent ion of 427.1 and a fragment ion of 139.1 at a CID of 25. Indomethacin (top plot) was not detected as measured by a parent ion of 357.9 and a fragment ion of 139.1 at a CID of 15.

FIGS. 23a-23i are a series of bar graphs showing that Compound A selectively increased brain eCBs without affecting related lipids. The effects of Compound A and PF-3845 on brain NAE levels two hours after i.p. injection are shown in FIGS. 23a and 23b, respectively. FIG. 23c shows the effect of PF-3845 alone, or in combination with Compound A, on brain NAE levels. FIG. 23d shows the effects of Compound A and PF-3 845 on liver NAE levels. FIG. 23e shows the effects of Compound A on brain AEA levels in WT and FAAH KO mice. The effects of Compound A and JZL-184 on brain MAG levels is shown in FIGS. 23f and 23g, respectively. FIG. 23h shows the effects of JZL-184 alone or in combination with Compound A on brain MAG levels. FIG. 23i shows the effects of Compound A on brain NAEs in WT and COX-2 KO mice. Multiplicity corrected p values and t-statistics by unpaired two-tailed t-tests with Holm-Sidak multiple comparisons a correction are shown in FIGS. 23a-23d and 23f-23h; F andp values for main effects of two-way ANOVA followed, and p values for Sidak's post hoc multiple comparisons test shown in FIG. 23e; F and p values for genotype X LM treatment interaction by two-way ANOVA, and p values for Sidak's multiple comparisons post hoc test for AEA levels shown in FIG. 23i. n=number of mice per treatment group indicated in bars. Error bars represent S.E.M.

FIGS. 24a-24e are a series of bar graphs showing that Compound A selectively increased AEA in peripheral tissues. The effects of Compound A on NAE, MAG, and PG levels in the stomach, small intestine, heart, kidney, and lung two hours after injection are shown in FIGS. 24a-24e, respectively. For comparison, the effects of indomethacin on PG levels are also shown for each tissue.

Multiplicity corrected p values and t-statistics by unpaired two-tailed t-tests with Holm-Sidak multiple comparisons a correction are given in each Figure. F and p values for one-way ANOVA, followed byp values from Dunnett's post hoc analysis are also given for PG analyses. n=number of mice per treatment group indicated. Error bars represent S.E.M.

FIGS. 25a-25h are a series of graphs showing that Compound A reduces anxiety-like behaviors in the novel open field. The effects of PF-3845 (FIG. 25a) and Compound A (FIG. 25b) on center distance and center time traveled in the open field over time are shown. FIGS. 25c-25e show the effects of Compound A on center distance and center time in the open field in WT and COX-2 KO mice. FIG. 25e is a bar graph summarizing the data for Compound A effects in WT and COX-2 KO mice on total center distance during the last 20 minutes of the assay. The effects of Compound A in vehicle (FIG. 25f) or Rimonabant (Rim) pretreated mice (FIG. 25g) are shown. FIG. 25h is a bar graph summarizing the data for Compound A effects after vehicle or Rim pretreatment on total center distance during the last 20 minutes of the assay. F and p values for drug effects by two-way repeated measures ANOVA given for FIGS. 25a-25d and 25f-25g; multiplicity corrected p values and t-statistics by unpaired two-tailed t-tests with Holm-Sidak multiple comparisons a correction given for center time figures for each significant time epoch in FIGS. 25a-25d and 25f-25g); F and p values for genotype X LM or LM X Rim pretreatment interaction given for FIG. 25e and FIG. 25h, respectively. Multiplicity corrected p values by Sidak's multiple comparisons post hoc test after ANOVA as indicated in FIGS. 25e and 25h). N=number of mice per treatment group. Error bars represent S.E.M.

FIGS. 26a-26c are a series of graphs showing the effects of COX inhibitors in the novel open-field test. FIG. 26a is a graph showing that indomethacin treatment significantly increased center distance. FIG. 26b is a graph showing that NS-398 significantly increased center distance. FIG. 26c is a graph showing that SC-560 had no significant behavioral effects.

FIG. 27 is a series of graphs comparing open-field tests of COX-2^{(-/-)} knockout and wild type mice. COX-2 KO animals spent significantly more time in the center of the open-field than WT littermates.

FIGS. 28a and 28b are a series of graphs showing the effects of Compound A in CB1^{(-/-)} mice. FIG. 28a shows that Compound A did not increase center distance or time in CB1^{(-/-)} mice. FIG. 28b shows that Compound A significantly increased AEA and 2-AG in CB1^{(-/-)} mice without affecting PG production.

FIGS. 29a-29f are a series of bar graphs showing that Compound A reduced anxiety behaviors in the light-dark box. FIGS. 29a-29c show the effects of PF-3845 on light zone time (FIG. 29a), light zone entries (FIG. 29b), and total distance travelled (FIG. 29c) in the light-dark box assay. FIGS. 29d-29f show the effects of Compound A with or without Rim pretreatment on parameters of the light-dark box assay. P values and t-statistics by unpaired two-tailed t-tests are given for FIGS. 29a-29c; F and p values for LM X Rim pretreatment interaction (INT) by twoway ANOVA shown in FIGS. 29d-29f; multiplicity corrected p values by Sidak's multiple comparisons post hoc test after ANOVA indicated in FIGS. 29d-29f. n=number of mice per treatment group indicated in the Figures. Error bars represent S.E.M.

FIGS. 30a-30f are a series of graphs showing the effects of Compound A and PF-3845 in an Elevated Plus Maze (EPM) test. FIGS. 30a-30c show that PF-3845 decreased the open arm latency but did not increase open arm time or total distance travelled in the EPM. FIGS. 30d-30f show that Compound A decreased the open-arm latency but did not increase open arm time or total distance travelled in the EPM.

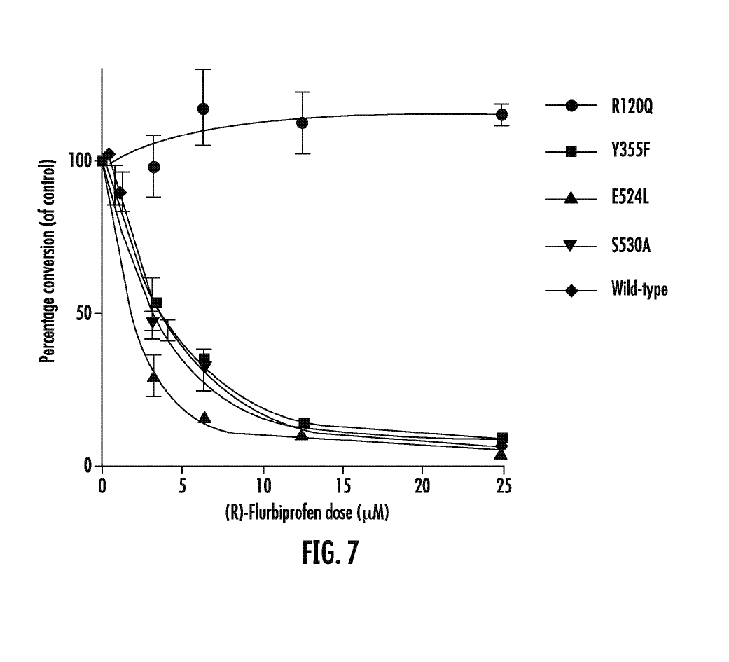

FIGS. 31a-31d are a series of graphs showing that Compound A did not exert overt cannabimimetic effects in vivo. FIG. 31a is a graph showing the effects of vehicle, Compound A, and Win 55212-2 on rectal temperature over time. FIG. 31b is a bar graph showing the effects of vehicle, Compound A, and Win 55212-2 on catalepsy over time. FIG. 31c is a bar graph showing the effects of vehicle, Compound A, and Win 55212-2 on anti-nociception in the hot plate test. FIG. 31d is a bar graph showing the effects of Compound A on novel object recognition. F and p values for drug treatment effects by two-way ANOVA are shown for FIGS. 31a and 31b, and for novelty recognition factor by two-way ANOVA in FIG. 7d; F and p values for drug effect by one-way ANOVA are shown for FIG. 31c. Multiplicity corrected p values by Sidak's multiple comparisons post hoc test (FIGS. 31a, 31b, and 31d) and by Dunnett's post hoc test (FIG. 31c) are shown. n=number of mice per treatment group indicated in figure. Error bars represent S.E.M.

FIG. 32 is a schematic representation of the relative substrate-selectivity of eCB degradation inhibitors. The left panel shows MAGL hydrolyzation of non-eCB MAGs and 2-AG to free fatty acids (FFAs) and AA, respectively. Inhibition of MAGL by JZL-184 increases MAG and 2-AG levels while reducing AA levels. The right panel shows FAAH hydrolyzation of AEA and other NAEs to AA and FFAs, respectively. Inhibition of FAAH by PF-3845 increases levels of AEA and other NAEs. The middle panel showsn COX-2 metabolism of 2-AG, AA, and AEA to PG-Gs, PGs, and PG-EAs, respectively. Inhibition of COX-2 by traditional non-SSICs increases levels of 2-AG (under some conditions), AA, and AEA, while reducing PG production. In contrast, SSIC by Compound A selectively increased eCBs without affecting non-eCB NAEs or MAGs. Importantly Compound A did not affect AA oxygenation to PGs and thus did not alter levels of AA or PG in the brain or periphery. Although not wishing to be bound by any particular theory of operation, this selectivity was presumptively due to the selectivity of COX-2 for AA-containing lipids over other fatty acids, whereas both FAAH and MAGL exhibited broad substrate utilization of a wide range of fatty-acid containing lipids with amide and glycerol-ester headgroups, respectively.

### DETAILED DESCRIPTION

The present subject matter will be now be described more fully hereinafter with reference to the accompanying Examples and Figures, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. Mention of techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, the phrase “a cell” refers to one or more cells, and can thus also refer to a tissue or an organ.

The term “about”, as used herein to refer to a measurable value such as an amount of weight, time, dose (e.g., therapeutic dose), etc., is meant to encompass in some embodiments variations of ±20%, in some embodiments ±10%, in some embodiments ±5%, in some embodiments ±1%, in some embodiments ±0.1%, in some embodiments ±0.5%, and in some embodiments ±0.01% from the specified amount, as such variations are appropriate to perform the disclosed methods.

As used herein, the term “and/or” when used in the context of a list of entities, refers to the entities being present singly or in any possible combination or subcombination. Thus, for example, the phrase “A, B, C, and/or D” includes A, B, C, and D individually, but also includes any and all combinations and subcombinations of A, B, C, and D.

As used herein, the term “COX-2” refers to a cyclooxygenase-2 gene or gene product. It is also referred to as prostaglandin-endoperoxide synthase 2 (PTGS2) and prostaglandin G/H synthase. Generally, COX-2 gene products catalyze the oxygenation of arachidonic acid (AA) and the endocannabinoids, 2-arachidonoylglycerol (2-AG) and arachidonoylethanolamide (AEA), to prostaglandin endoperoxide derivatives. COX-2 gene products have been identified in several species, and biosequences corresponding thereto are present in the GENBANK® database. By way of example and not limitation, in some embodiments a COX-2 gene product comprises, consists essentially of, and/or consists of a sequence as set forth in Table 1.

TABLE 1
GENBANK ® Database Accession Nos. for Exemplary
COX-2 Biosequences

| | Nucleotide | Amino Acid |
|---|---|---|
| Species | Sequence | Sequence |
| Homo sapiens | NM_000963 | NP_000954 |
| Mus musculus | NM_011198 | NP_035328 |
| Rattus norvegicus | NM_017232 | NP_058928 |
| Felis catus | NM_001110449 | NP_001103919 |
| Sus scrofa | NM_214321 | NP_999486 |
| Canis lupus familiaris | NM_001003354 | NP_001003354 |
| Equus caballus | NM_001081775 | NP_001075244 |
| Xenopus laevis | NM_001093477 | NP_001086946 |

As used herein, the term “cell” refers not only to the particular subject cell (e.g., a living biological cell), but also to the progeny or potential progeny of such a cell. Because certain modifications can occur in succeeding generations due to either mutation or environmental influences, such progeny cells might not, in fact, be identical to the parent cell, but are still included within the scope of the term as used herein.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. For example, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a therapeutic method of the presently disclosed subject matter can “consist essentially of” one or more enumerated steps as set forth herein, which means that the one or more enumerated steps produce most or substantially all of the therapeutic benefit intended to be produced by the claimed method. It is noted, however, that additional steps can be encompassed within the scope of such a therapeutic method, provided that the additional steps do not substantially contribute to the therapeutic benefit for which the therapeutic method is intended.

In some embodiments, the pharmaceutical compositions of the presently disclosed subject matter consist essentially of an SSCI and a pharmaceutically acceptable carrier or excipient. In some embodiments, the SSCI consists essentially of a substantially pure (R)-profen or a derivative thereof, optionally wherein the derivative thereof does not stereoisomerize in vivo to an (S)-profen. In some embodiments, the (R)-profen or a derivative thereof is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

With respect to the terms “comprising”, “consisting essentially of”, and “consisting of”, where one of these three terms is used herein, the presently disclosed and claimed subject matter can include the use of either of the other two terms. For example, the presently disclosed subject matter relates in some embodiments to compositions that comprise the SSCIs disclosed herein. It is understood that the presently disclosed subject matter thus also encompasses compositions that in some embodiments consist essentially of the SSCIs disclosed herein, as well as compositions that in some embodiments consist of the SSCIs disclosed herein. Similarly, it is also understood that in some embodiments the methods of the presently disclosed subject matter comprise the steps that are disclosed herein, in some embodiments the methods of the presently disclosed subject matter consist essentially of the steps that are disclosed, and in some embodiments the methods of the presently disclosed subject matter consist of the steps that are disclosed herein.

As used herein, the term “enzyme” refers to a polypeptide that catalyzes a transformation of a substrate into a product at a rate that is substantially higher than occurs in a non-enzymatic reaction.

As used herein the term “gene” refers to a hereditary unit including a sequence of DNA that occupies a specific location on a chromosome and that contains the genetic instruction for a particular characteristic or trait in an organism.

Similarly, the phrase “gene product” refers to biological molecules that are the transcription and/or translation products of genes. Exemplary gene products include, but are not limited to mRNAs and polypeptides that result from translation of mRNAs. As would be understood by those of ordinary skill, gene products can also be manipulated in vivo or in vitro using well known techniques, and the manipulated derivatives can also be gene products. For example, a cDNA is an enzymatically produced derivative of an RNA molecule (e.g., an mRNA), and a cDNA is considered a gene product. Additionally, polypeptide translation products of mRNAs can be enzymatically fragmented using techniques well known to those of skill in the art, and these peptide fragments are also considered gene products.

As used herein, the term “inhibitor” refers to a chemical substance that inactivates or decreases the biological activity of a polypeptide (e.g., an enzymatic activity). In some embodiments, the polypeptide is a cyclooxygnease (COX) polypeptide (e.g., a COX-1 polypeptide or a COX-2 polypeptide). In some embodiments, the biological activity of the COX polypeptide catalyzes the metabolism of arachidonic acid (AA) to prostaglandin H2 (PGH2). In some embodiments, the biological activity of the COX polypeptide catalyzes the oxygenation of the endocannabinoids 2-arachidonoylglycerol (2-AG) and arachidonoylethanolamide (AEA) to prostaglandin endoperoxide derivatives.

As used herein, the term “interact” includes “binding” interactions and “associations” between molecules. Interactions can be, for example, protein-protein, protein-small molecule, protein-nucleic acid, and nucleic acid-nucleic acid in nature.

As used herein, the term “modulate” refers to an increase, decrease, or other alteration of any, or all, chemical and biological activities or properties of a biochemical entity, e.g., a wild type or mutant polypeptide. As such, the term “modulate” can refer to a change in the expression level of a gene (or a level of RNA molecule or equivalent RNA molecules encoding one or more proteins or protein subunits), or of an activity of one or more proteins or protein subunits, such that expression, level, or activity is greater than or less than that observed in the absence of the modulator. For example, the term “modulate” can mean “inhibit” or “suppress”, but the use of the word “modulate” is not limited to this definition.

The term “modulation” as used herein refers to both upregulation (i.e., activation or stimulation) and downregulation (i.e., inhibition or suppression) of a response. Thus, the term “modulation”, when used in reference to a functional property or biological activity or process (e.g., enzyme activity or receptor binding), refers to the capacity to upregulate (e.g., activate or stimulate), downregulate (e.g., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like (naturally occurring or non-naturally occurring) that can be capable of causing modulation. Modulators can be evaluated for potential activity as inhibitors or activators (directly or indirectly) of a functional property, biological activity or process, or a combination thereof, (e.g., agonist, partial antagonist, partial agonist, inverse agonist, antagonist, and the like) by inclusion in assays. In such assays, many modulators can be screened at one time. The activity of a modulator can be known, unknown, or partially known.

Modulators can be either selective or non-selective. As used herein, the term “selective” when used in the context of a modulator (e.g., an inhibitor) refers to a measurable or otherwise biologically relevant difference in the way the modulator interacts with one molecule (e.g., a COX-1 polypeptide) versus another similar but not identical molecule (e.g., a COX-2 polypeptide).

It must be understood that it is not required that the degree to which the interactions differ be completely opposite. Put another way, the term selective modulator encompasses not only those molecules that only bind to a given polypeptide (e.g., COX-2) and not to related family members (e.g., COX-1, or vice versa). The term is also intended to include modulators that are characterized by interactions with polypeptides of interest and from related family members that differ to a lesser degree. For example, selective modulators include modulators for which conditions can be found (such as the nature of the substituents present on the modulator) that would allow a biologically relevant difference in the binding of the modulator to the polypeptide of interest (e.g., COX-2) versus polypeptides derived from different family members (e.g., COX-1).

When a selective modulator is identified, the modulator will bind to one molecule (for example, COX-2) in a manner that is different (for example, stronger) than it binds to another molecule (for example, COX-1). As used herein, the modulator is said to display “selective binding” or “preferential binding” to the molecule to which it binds more strongly.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. For example, a p-value less than or equal to in some embodiments 0.05, in some embodiments less than 0.01, in another example less than 0.005, and in yet another example less than 0.001, are regarded as significant.

As used herein, the term “significant increase” refers to an increase in activity (for example, enzymatic activity) that is larger than the margin of error inherent in the measurement technique, in some embodiments an increase by about 2 fold or greater over a baseline activity (for example, the activity of the wild type enzyme in the presence of an activator), in some embodiments an increase by about fold or greater, and in still some embodiments an increase by about 10 fold or greater.

With respect to the binding of one or more molecules (for example, a modulator) to one or more polypeptides (for example, a COX polypeptide), a significant increase can also refer to: (a) a biologically relevant difference in binding of two or more related compounds to the same polypeptide; and/or (b) a biologically relevant difference in binding of the same compound to two different polypeptides. In this aspect, “significant” is to be thought of in its ordinary meaning: namely, a difference between two occurrences that is important (i.e., biologically or medically relevant). By way of example, a significant increase can also refer to an increase in the amount of a derivative of an NSAID (for example, an NSAID derivative of the presently disclosed subject matter) that interacts with a particular COX polypeptide (for example, a COX-2 polypeptide) per unit dose of the derivative administered as compared to the amount of the non-derivatized NSAID that interacts with the same COX polypeptide per unit dose of the non-derivatized NSAID. In some embodiments, if a derivative binds to a particular COX enzyme less strongly than does the parent NSAID from which is was derived, on a mole-for-mole basis, more of the derivative should be available to interact with other COX polypeptides than would be available if the parent NSAID were administered.

As used herein, the terms “significantly less” and “significantly reduced” refer to a result (for example, an amount of a product of an enzymatic reaction or an extent of binding to a target such as, but not limited to a cyclooxygenase) that is reduced by more than the margin of error inherent in the measurement technique, in some embodiments a decrease by about 2 fold or greater with respect to a baseline activity (for example, the baseline activity of the enzyme in the absence of the inhibitor), in some embodiments, a decrease by about 5 fold or greater, and in still some embodiments a decrease by about 10 fold or greater.

The term “subject” as used herein refers to a member of any invertebrate or vertebrate species. Accordingly, the term “subject” is intended to encompass any member of the Kingdom Animalia including, but not limited to the phylum Chordata (i.e., members of Classes Osteichythyes (bony fish), Amphibia (amphibians), Reptilia (reptiles), Aves (birds), and Mammalia (mammals)), and all Orders and Families encompassed therein.

Similarly, all genes, gene names, and gene products disclosed herein are intended to correspond to orthologs from any species for which the compositions and methods disclosed herein are applicable. Thus, the terms include, but are not limited to genes and gene products from humans and other mammals. It is understood that when a gene or gene product from a particular species is disclosed, this disclosure is intended to be exemplary only, and is not to be interpreted as a limitation unless the context in which it appears clearly indicates. Thus, for example, the genes and/or gene products disclosed herein are intended to encompass homologous genes and gene products from other animals including, but not limited to other mammals, fish, amphibians, reptiles, and birds.

The methods and compositions of the presently disclosed subject matter are particularly useful for warm-blooded vertebrates. Thus, the presently disclosed subject matter concerns mammals (including, but not limited to humans) and birds. More particularly provided is the use of the methods and compositions of the presently disclosed subject matter on mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the application of the methods and compositions of the presently disclosed subject matter to livestock, including but not limited to domesticated swine (pigs and hogs), ruminants, horses, poultry, and the like. As used herein, the phrase “substantially pure” refers to a compound or composition that is in some embodiments greater than 50% pure, in some embodiments greater than 60% pure, in some embodiments greater than 70% pure, in some embodiments greater than 80% pure, in some embodiments greater than 90% pure, in some embodiments greater than 95% pure, in some embodiments greater than 96% pure, in some embodiments greater than 97% pure, in some embodiments greater than 98% pure, and in some embodiments greater than 99% pure. In some embodiments, the purity of an enantiomeric compound in a composition is expressed as compared to the amount of the other enantiomer that is present in the composition. For example, a “substantially pure (R)-profen” is in various embodiments greater than 50%, 60%, 70%, 75%, 80%, 85%, 90%, 95%, 96%, 97%, 98%, or 99% pure with respect to any (S)-profen that might be present in the composition.

As used herein, the phrases “substrate-selective COX-2 inhibitor” (SSCI) and “substrate-selective inhibitor of endocannabinoid oxygenation by COX-2” are used interchangeably to refer to a compound and/or composition that in some embodiments is a weak, competitive inhibitor of arachidonic acid oxygenation by COX-2, but in some embodiments acts as a potent non-competitive inhibitor of 2-AG and AEA oxygenation by COX-2. In some embodiments, an SSCI is selected from the group consisting of a substantially pure (R)-profen or a derivative thereof, optionally wherein the derivative thereof does not stereoisomerize in vivo to an (S)-profen. In some embodiments, the substrate-selective inhibitor of endocannabinoid oxygenation of COX-2 is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), ibuprofen, naproxen, lumiracoxib, derivatives thereof, and anlogues thereof.

### II. COMPOSITIONS

II.A. General Considerations

In some embodiments, the presently disclosed subject matter provides compounds that are SSCIs. In some embodiments, the SSCIs are substantially pure (R)-profens or derivatives thereof, optionally wherein the derivative thereof does not stereoisomerize in vivo to an (S)-profen. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

Throughout the specification, Figures, and claims, some structural formulas are depicted without including certain methyl groups and/or hydrogens. In the structural formulas, solid lines represent bonds between two atoms, and unless otherwise indicated, between carbon atoms. Thus, bonds that have no atom specifically recited on one end and/or the other have a carbon atom at that and/or the other end. For example, a structural formula depicted as “—O—” represents C—O—C. Given that hydrogens are not explicitly placed in all structural formulas, implicit hydrogens are understood to exist in the structural formulas as necessary. Thus, a structural formula depicted as “—O” can represent H₃C—O, as appropriate given the valences of the particular atoms.

As used herein, the term “alkyl” means in some embodiments C₁₋₁₀ inclusive (i.e., carbon chains comprising 1, 2, 3, 4, 5, 6, 7, 8, 9, or 10 carbon atoms); in some embodiments C₁₋₆ inclusive (i.e., carbon chains comprising 1, 2, 3, 4, 5, or 6 carbon atoms); and in some embodiments C₁₋₄ inclusive (i.e., carbon chains comprising 1, 2, 3, or 4, carbon atoms) linear, branched, or cyclic, saturated or unsaturated (i.e., alkenyl and alkynyl) hydrocarbon chains, including for example, methyl, ethyl, propyl, isopropyl, butyl, isobutyl, tert-butyl, pentyl, hexyl, ethenyl, propenyl, butenyl, pentenyl, hexenyl, butadienyl, propynyl, butynyl, pentynyl, hexynyl, and allenyl groups.

The alkyl group can be optionally substituted with one or more alkyl group substituents, which can be the same or different, where “alkyl group substituent” includes alkyl, halo, arylamino, acyl, hydroxy, aryloxy, alkoxyl, alkylthio, arylthio, aralkyloxy, aralkylthio, carboxy, alkoxycarbonyl, oxo, and cycloalkyl. In this case, the alkyl can be referred to as a “substituted alkyl”. Representative substituted alkyls include, for example, benzyl, trifluoromethyl, and the like. There can be optionally inserted along the alkyl chain one or more oxygen, sulfur or substituted or unsubstituted nitrogen atoms, wherein the nitrogen substituent is hydrogen, alkyl (also referred to herein as “alkylaminoalkyl”), or aryl. Thus, the term “alkyl” can also include esters and amides. “Branched” refers to an alkyl group in which an alkyl group, such as methyl, ethyl, or propyl, is attached to a linear alkyl chain.

The term “aryl” is used herein to refer to an aromatic substituent, which can be a single aromatic ring or multiple aromatic rings that are fused together, linked covalently, or linked to a common group such as a methylene or ethylene moiety. The common linking group can also be a carbonyl as in benzophenone or oxygen as in diphenylether or nitrogen in diphenylamine. The aromatic ring(s) can include phenyl, naphthyl, biphenyl, diphenylether, diphenylamine, and benzophenone among others. In particular embodiments, the term “aryl” means a cyclic aromatic comprising about 5 to about 10 carbon atoms, including 5 and 6-membered hydrocarbon and heterocyclic aromatic rings.

An aryl group can be optionally substituted with one or more aryl group substituents which can be the same or different, where “aryl group substituent” includes alkyl, aryl, aralkyl, hydroxy, alkoxyl, aryloxy, aralkoxyl, carboxy, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene and —NR′R″, where R′ and R″ can be each independently hydrogen, alkyl, aryl and aralkyl. In this case, the aryl can be referred to as a “substituted aryl”. Also, the term “aryl” can also include esters and amides related to the underlying aryl group.

Specific examples of aryl groups include but are not limited to cyclopentadienyl, phenyl, furan, thiophene, pyrrole, pyran, pyridine, imidazole, isothiazole, isoxazole, pyrazole, pyrazine, pyrimidine, and the like.

The term “alkoxy” is used herein to refer to the —OZ¹ radical, where Z¹ is selected from the group consisting of alkyl (in some embodiments, C₁ to C₆ alkyl), substituted alkyl, cycloalkyl, substituted cycloalkyl, heterocycloalkyl, substituted heterocycloalkyl, silyl groups, and combinations thereof as described herein. Suitable alkoxy radicals include, for example, methoxy, ethoxy, benzyloxy, t-butoxy, etc. A related term is “aryloxy” where Z¹ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy, and the like.

The term “amino” is used herein to refer to the group —NZ¹Z², where each of Z¹ and Z² is independently selected from the group consisting of hydrogen; alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof. Additionally, the amino group can be represented as N⁺Z¹ Z² Z³, with the previous definitions applying and Z³ being either H or alkyl.

As used herein, the term “acyl” refers to an organic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Aroyl” means an aryl-CO— group wherein aryl is as previously described. Exemplary aroyl groups include benzoyl and 1- and 2-naphthoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, lower alkyl, or aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Aralkyl” refers to an aryl-alkyl-group wherein aryl and alkyl are as previously described. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Dialkylamino” refers to an —NRR′ group wherein each of R and R′ is independently an alkyl group as previously described. Exemplary alkylamino groups include ethylmethylamino, dimethylamino, and diethylamino.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an H₂N—CO— group.

“Alkylcarbamoyl” refers to a R′RN—CO— group wherein one of R and R′ is hydrogen and the other of R and R′ is alkyl as previously described.

“Dialkylcarbamoyl” refers to a R′RN—CO— group wherein each of R and R′ is independently alkyl as previously described.

“Acyloxyl” refers to an acyl-O— group wherein acyl is as previously described.

“Acylamino” refers to an acyl-NH— group wherein acyl is as previously described.

“Aroylamino” refers to an aroyl-NH— group wherein aroyl is as previously described.

The term “amino” refers to the —NH₂ group.

The term “carbonyl” refers to the —(C═O)— group.

The term “carboxyl” refers to the —COOH group.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described herein wherein a carbon or oxygen atom is replaced by a sulfur atom.

The term “sulfate” refers to the —SO₄ group.

In some embodiments, an SSCI of the presently disclosed subject matter is an (R)-profen. As used herein, the term “(R)-profen” refers to a derivative of 2-phenylpropanoic acid that has a chiral center and is capable of binding to and modulating a biological activity of a COX-2 enzyme. Exemplary, non-limiting (R)-profens are disclosed herein.

In some embodiments, the (R)-profen or (R)-profen derivative has a structure:

Ar—Z—CO₂H,

wherein:

Ar is aryl, optionally substituted by one or more aryl group substituents; and

Z is methylene, mono-substituted methylene (i.e., —CH(Z′)—, wherein Z′ is an alkyl group substituent), or di-substituted methylene (i.e., —C(Z′)₂—, wherein each Z′ is an alkyl group substituent or both Z′ groups together can form an alkylene group).

In some embodiments, the carboxylic acid group of the profen or profen derivative can be replaced by an ester or amide (e.g., that can be hydrolyzed in vivo or in vitro to provide the carboxylic acid group).

In some embodiments, Z is selected from methylene, alkyl-substituted methylene (e.g., —CH(CH₃)—), dialkyl-substituted methylene (e.g., —C(CH₃)₂—), and

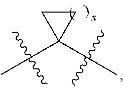

wherein x is an integer between 1 and 10. In some embodiments, x is an integer between 1 and 5 (i.e., 1, 2, 3, 4, or 5). In some embodiments, x is 1.

In some embodiments, Z is

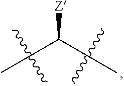

wherein Z′ is alkyl. In some embodiments, Z′ is methyl.

In some embodiments, Ar is phenyl or napthyl, optionally substituted with one or more aryl group substituents (e.g., halo, aryl, alkyl, alkoxyl, aryloxy (e.g., phenoxy), and acyl (e.g., aroyl)). In some embodiments, Ar is diphenyl (e.g., phenyl-substituted phenyl, optionally substituted with one or more additional aryl group substituents). In some embodiments, Ar is selected from the group comprising:

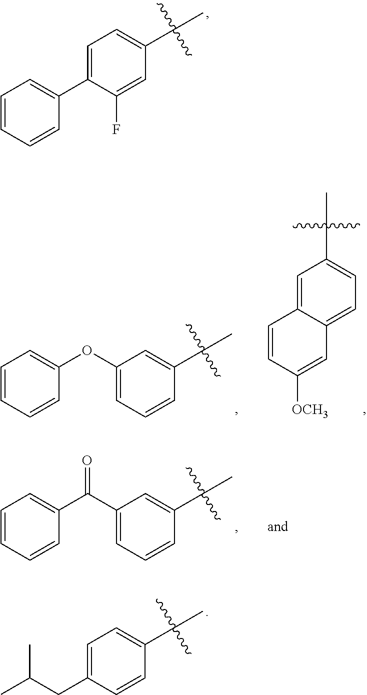 [SMILES: *c1ccc(-c2ccccc2)c(F)c1 ; *c1cccc(Oc2ccccc2)c1 ; *c1ccc2cc(OC)ccc2c1 ; *c1cccc(C(=O)c2ccccc2)c1 ; *c1ccc(CC(C)C)cc1]

In some embodiments, an SSCI is an indomethacin analog. In some embodiments, the indomethancin analog has the structure:

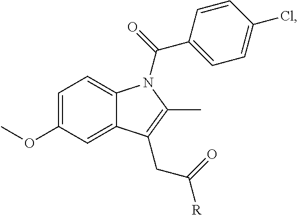

wherein R is selected from —NH₂, —NHR′, and —N(R″)₂, wherein R′ can be selected from alkyl, substituted alkyl (e.g., hydroxyl substituted alkyl), aralkyl, aryl, and substituted aryl; and each R″ can be independently selected from alkyl, substituted alkyl, aralkyl, aryl, or substituted aryl or wherein the two R″ groups together form an alkylene group, wherein said alkylene group can be optionally substituted or include a heteroatom (e.g., O or S) in the alkylene chain in place of a carbon atom).

In some embodiments, R is —N(R″)₂.

In some embodiments, an SSCI is a Lumiracoxib or a derivative thereof. In some embodiments, the Lumiracoxib or derivative thereof has the structure:

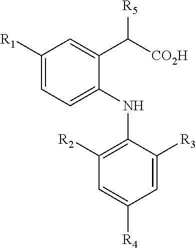

wherein:

each of R₁, R₂, R₃, and R₄ is selected from the group comprising H, alkyl, aryl, aralkyl, alkoxyl, aryloxy, aralkoxyl, carboxy, acyl, halo (i.e., fluoro, chloro, bromo or iodo), mercapto, hydroxyl, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, amino, alkylamino and dialkylamino; and Rs is selected from H, alkyl, and halo. In some embodiments, the carboxylic acid group of the Lumiracoxib or derivative thereof can be replaced by an ester or amide (e.g., that can be hydrolyzed in vivo or in vitro to provide the carboxylic acid group).

In some embodiments, R₅ is H and the Lumiracoxib or derivative thereof has the structure:

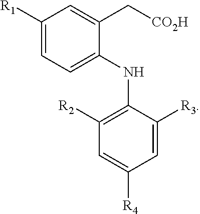

In some embodiments, R₁ is selected from H, alkyl, and halo. In some embodiments, R₁ is selected from H and alkyl. In some embodiments, R₁ is H or methyl. In some embodiments, R₁ is methyl.

In some embodiments, each of R₂, R₃, and R₄ is selected from H, alkyl, and halo. In some embodiments, each of R₂, R₃, and R₄ is selected from H, methyl, and halo.

In some embodiments, at least one of R₂, R₃, and R₄ is halo. In some embodiments, at least two of R₂, R₃, and R₄ are halo. In some embodiments, at least R₂ and R₃ are halo.

In some embodiments, R₁ is alkyl (e.g., methyl) and at least one of R₂, R₃, and R₄ is halo.

II.B. Development and Evaluation of Achiral Profen Probes

Cyclooxygenase-2 (COX-2) oxygenates arachidonic acid and the endocannabinoids, 2-arachidonoylglycerol (2-AG) and arachidonoylethanolamide (AEA). (R)-profens selectively inhibit endocannabinoid oxygenation but not arachidonic acid oxygenation. This was evaluated by synthesizing achiral derivatives of five profen scaffolds and evaluating them for substrate-selective inhibition using in vitro and cellular assays. The size of the substituents dictated the inhibitory strength of the analogs, with smaller substituents enabling greater potency but less selectivity. Inhibitors based on the flurbiprofen scaffold possessed the greatest potency and selectivity, with desmethylflurbiprofen (Compound 3a) exhibiting an IC₅₀ of 0.11 μM for inhibition of 2-AG oxygenation. The crystal structure of desmethylflurbiprofen complexed to mCOX-2 demonstrated a similar binding mode to other profens. Desmethylflurbiprofen exhibited a half-life in mice comparable to that of ibuprofen. The data presented suggest that achiral profens can act as lead molecules toward in vivo probes of substrate-selective COX-2 inhibition.

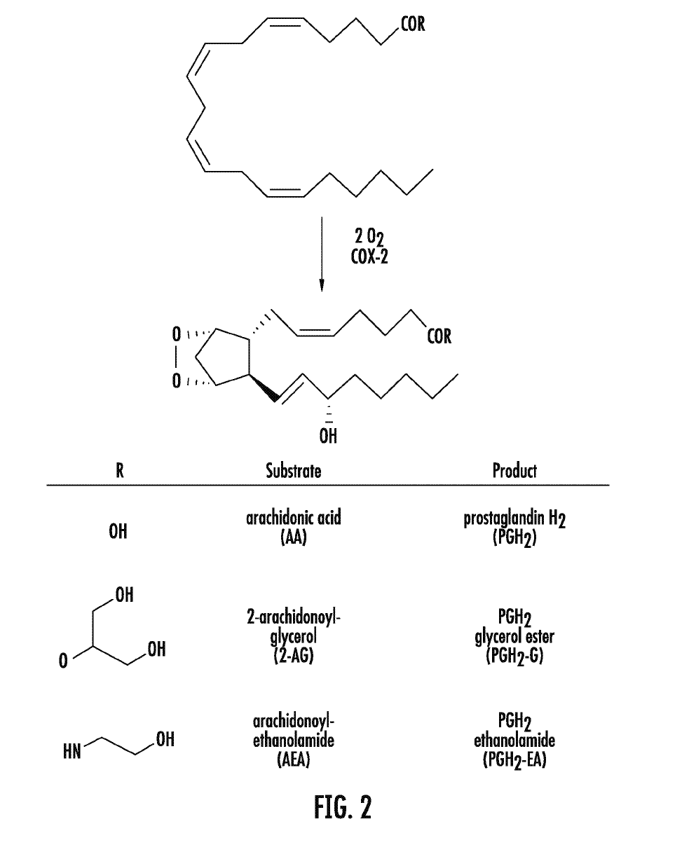

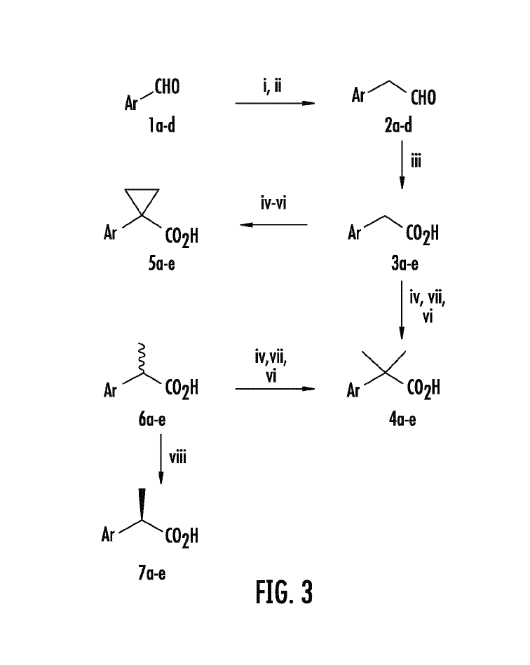

Cyclooxygenase-2 (COX-2) is a molecular target for non-steroidal anti-inflammatory drugs (NSAIDs). It generates prostaglandin-H₂ (PGH₂), PGH₂-glyceryl ester (PGH₂-G) and PGH₂-ethanolamide (PGH₂-EA) from arachidonic acid (AA) and the endocannabinoids, 2-arachidonoylglycerol (2-AG) and arachidonoylethanolamide (AEA), respectively (see FIG. 2; Turini et al, 2002; Rouzer et al., 2011). Metabolism of PGH₂ generates PG's that function in inflammation, vascular homeostasis and gastric cytoprotection (Funk et al., 2001; Rouzer et al., 2009). The biological functions of the PG derivatives of PGH₂-Gs and PGH₂-EAs are largely unknown, but they have been implicated in unique roles in macrophages, tumor cells and neurons (Sang et al., 2007; Woodward et al., 2008). In addition to serving as precursors to PG esters and amides, 2-AG and AEA act as agonists of the cannabinoid (CB1 and CB2) receptors (Svizenska et al., 2008). CB1 receptors have primarily been studied for the analgesic, locomotor, and temperature regulatory effects. Additionally, CB1 and CB2 receptors are involved in neuroprotection, modulation of inflammation, and carcinogenesis (Wang et al., 2008; Fowler et al., 2010; Nomura et al., 2011). Thus, COX-2 oxygenation of 2-AG and AEA might lower cannabinoid tone by reducing the concentrations of endocannabinoids and might generate new bioactive lipids by increasing the concentrations of PG esters and amides (Kozak et al., 2001; Ueda et al., 2011).

Rapid, reversible inhibitors of COX-2, such as ibuprofen and mefenamic acid, are significantly more potent inhibitors of 2-AG and AEA oxygenation than AA oxygenation. These “substrate-selective” inhibitors bind in one active site of the COX-2 homodimer and alter the structure of the second active site so that 2-AG and AEA oxygenation is inhibited, but AA oxygenation is not. Inhibition of AA oxygenation requires rapid, reversible inhibitors to bind in both active sites. Inhibitor binding in the second active site requires much higher concentrations than binding in the first active site, which gives rise to the phenomenon of substrate-selective inhibition. (R)-enantiomers of the arylpropionic acid (profen) class of NSAIDs, which were considered to be inactive as COX inhibitors, have been shown to be substrate-selective.

In some embodiments, the presently disclosed subject matter provides in vivo probes that can be employed to determine the effects of blocking 2-AG and AEA, but not AA, oxygenation by COX-2.

### III. EXEMPLARY SYNTHESIS METHODS

In some embodiments, the presently disclosed subject matter provides methods for producing SSCIs. It is noted that any suitable synthesis scheme can be employed for producing the presently disclosed SSCIs, and one of ordinary skill in the art will understand what synthesis schemes can be employed based on the exemplary compounds disclosed herein.

Representative synthesis schemes are discussed in more detail herein below in the EXAMPLES and the Figures. It is understood that the representative schemes are non-limiting, and further that the scheme depicted in Scheme 1 as being applicable for synthesizing achiral profens can also be employed with modifications that would be apparent to one of ordinary skill in the art after review of the instant specification for synthesizing additional derivatives that fall within the scope of the instant disclosure.

### IV. METHODS FOR MODULATING COX POLYPEPTIDE BIOLOGICAL ACTIVITIES

Also provided herein are methods for using the disclosed SSCIs to modulate COX-2 polypeptide biological activities. In some embodiments, the methods comprise contacting a COX-2 polypeptide with an effective amount of a compound as disclosed herein including, but not limited to those disclosed herein.

The presently disclosed subject matter also provides methods for selectively inhibiting endocannabinoid oxygenation but not arachidonic acid oxygenation via COX-2. In some embodiments, the methods comprising contacting a COX-2 polypeptide with an effective amount of an SSCI as disclosed herein. Exemplary, non-limiting SSCIs include substantially pure (R)-profens or derivatives thereof, optionally wherein the derivatives thereof do not stereoisomerize in vivo to (S)-profens. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and combinations thereof. The presently disclosed subject matter also provides methods for elevating local endogenous cannabinoid concentrations in tissues, cells, organs, and/or structures in a subject. In some embodiments, the methods comprise contacting a COX-2 polypeptide present in the subject with an effective amount of an SSCI as disclosed herein. Exemplary, non-limiting SSCIs include substantially pure (R)-profens or derivatives thereof, optionally wherein the derivatives thereof do not stereoisomerize in vivo to (S)-profens. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and combinations thereof.

In some embodiments, the presently disclosed subject matter also provides methods for reducing depletion of endogenous cannabinoids in tissues, cells, organs, and/or structures in subjects. In some embodiments, the methods comprise contacting a COX-2 polypeptide present in a subject with an effective amount of an SSCI as disclosed herein. In some embodiments, the COX-2 polypeptide is present in a tissue, cell, organ, and/or structure in a subject and/or is present in a distant location in the subject that under normal conditions provides an endogenous cannabinoid to the tissue, cell, organ, and/or structure in the subject. Exemplary, non-limiting SSCIs include substantially pure (R)-profens or derivatives thereof, optionally wherein the derivatives thereof do not stereoisomerize in vivo to (S)-profens. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and combinations thereof. In some embodiments, the COX-2 polypeptide is present in a region of inflammation in the subject.

The presently disclosed subject matter also provides methods for inducing analgesia in subjects. In some embodiments, the methods comprise contacting a COX-2 polypeptide present in the subject with an effective amount of a substrate-selective COX-2 inhibitor. In some embodiments, the SSCI comprises a substantially pure (R)-profen or a derivative thereof, optionally wherein the derivative thereof does not stereoisomerize in vivo to an (S)-profen. Exemplary, non-limiting SSCIs include substantially pure (R)-profens or derivatives thereof, optionally wherein the derivatives thereof do not stereoisomerize in vivo to (S)-profens. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and combinations thereof. In some embodiments, the COX-2 polypeptide is present in a region of inflammation in the subject. In some embodiments, the COX-2 polypeptide is present in a region of inflammation in the subject.

The presently disclosed subject matter also provides methods for providing an anxiolytic therapy, antidepressant therapy, or both to subjects. In some embodiments, the methods comprise contacting a COX-2 polypeptide present in the subject with an effective amount of an SSCI as disclosed herein. In some embodiments, the SSCI comprises a substantially pure (R)-profen or a derivative thereof, optionally wherein the derivative thereof does not stereoisomerize in vivo to an (S)-profen. Exemplary, non-limiting SSCIs include substantially pure (R)-profens or derivatives thereof, optionally wherein the derivatives thereof do not stereoisomerize in vivo to (S)-profens. In some embodiments, the SSCI is selected from the group consisting of Compounds A-G, Compounds 3a-3e, Compounds 4a-4e, Compounds 5a-5e, Compounds 7a-7e, Compounds 101-116, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and combinations thereof. In some embodiments, the COX-2 polypeptide is present in a region of inflammation in the subject.

The methods disclosed herein for using the disclosed SSCIs to modulate, optionally inhibit, COX-2 polypeptide biological activities or subsets thereof including, but not limited to endocannabinoid oxygenation, can be used for in vivo, ex vivo, and/or in vitro modulation, optionally inhibition, of COX-2 polypeptide biological activities and/or subsets thereof. As such, in some embodiments the COX-2 polypeptide is present within a subject, optionally wherein the subject is a mammal, including but not limited to a human.

IV.A. Formulations

A composition that comprises, consists essentially of, or consists of an SSCI as described herein comprises in some embodiments a composition that includes a pharmaceutically acceptable carrier. Suitable formulations include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents.

The compositions used in the methods can take such forms as suspensions, solutions, or emulsions in oily or aqueous vehicles, and can contain formulatory agents such as suspending, stabilizing, and/or dispersing agents. The compositions used in the methods can take forms including, but not limited to peroral, intravenous, intraperitoneal, inhalation, intraprostatic, and intratumoral formulations. Alternatively or in addition, the active ingredient can be in powder form for constitution with a suitable vehicle (e.g., sterile pyrogen-free water) before use.

The formulations can be presented in unit-dose or multi-dose containers, for example sealed ampules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulfate). The tablets can be coated by methods known in the art. For example, a neuroactive steroid can be formulated in combination with hydrochlorothiazide, and as a pH stabilized core having an enteric or delayed-release coating which protects the neuroactive steroid until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring, and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

In some embodiments, the presently disclosed subject matter employs a COX inhibitor composition that is pharmaceutically acceptable for use in humans. One of ordinary skill in the art understands the nature of those components that can be present in a COX inhibitor composition that is pharmaceutically acceptable for use in humans and also what components should be excluded from a COX inhibitor composition that is pharmaceutically acceptable for use in humans.

IV.B. Doses

As used herein, the phrases “treatment effective amount”, “therapeutically effective amount”, “treatment amount”, and “effective amount” are used interchangeably and refer to an amount of a therapeutic composition sufficient to produce a measurable response (e.g., a biologically or clinically relevant response in a subject being treated). Actual dosage levels of active ingredients in the pharmaceutical compositions of the presently disclosed subject matter can be varied so as to administer an amount of the active compound(s) that is effective to achieve the desired therapeutic response for a particular subject. The selected dosage level can depend upon the activity of the therapeutic composition, the route of administration, combination with other drugs or treatments, the severity of the condition being treated, the condition and prior medical history of the subject being treated, etc. However, it is within the skill of the art to start doses of the compound at levels lower than required to achieve the desired therapeutic effect and to gradually increase the dosage until the desired effect is achieved.

The potency of a therapeutic composition can vary, and therefore a “therapeutically effective amount” can vary. However, one skilled in the art can readily assess the potency and efficacy of a candidate modulator of the presently disclosed subject matter and adjust the therapeutic regimen accordingly.

After review of the disclosure herein of the presently disclosed subject matter, one of ordinary skill in the art can tailor the dosages to an individual subject, taking into account the particular formulation, method of administration to be used with the composition, and other factors. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

Thus, in some embodiments the term “effective amount” is used herein to refer to an amount of an SSCI, a pharmaceutically acceptable salt thereof, a derivative thereof, or a combination thereof sufficient to produce a measurable an amelioration of a symptom and/or consequence associated with undesirable COX (e.g., COX-2) biological activity. Actual dosage levels of active ingredients in an SSCI composition of the presently disclosed subject matter can be varied so as to administer an amount of the active compound(s) that is effective to achieve the desired response for a particular subject and/or application. The selected dosage level can depend upon a variety of factors including the activity of the SSCI composition, formulation, route of administration, combination with other drugs or treatments, severity of the condition being treated, and physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are known to those of ordinary skill in the art.

For administration of an SSCI composition as disclosed herein, conventional methods of extrapolating human dosage based on doses administered to a murine animal model can be carried out using techniques known to one of ordinary skill in the art. Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al., 1966. Briefly, to express a mg/kg dose in any given species as the equivalent mg/m² dose, multiply the dose by the appropriate km factor. In an adult human, 100 mg/kg is equivalent to 100 mg/kg 37 kg/m 2=3700 mg/m².

For additional guidance regarding formulations and doses, see U.S. Pat. Nos. 5,326,902; 5,234,933; PCT International Publication No. WO 93/25521; Remington et al., 1975; Goodman et al., 1996; Berkow et al., 1997; Speight et al., 1997; Ebadi, 1998; Duch et al., 1998; Katzung, 2001; Gerbino, 2005.

IV.C. Routes of Administration

The presently disclosed compositions can be administered to a subject in any form and/or by any route of administration. In some embodiments, the formulation is a sustained release formulation, a controlled release formulation, or a formulation designed for both sustained and controlled release. As used herein, the term “sustained release” refers to release of an active agent such that an approximately constant amount of an active agent becomes available to the subject over time. The phrase “controlled release” is broader, referring to release of an active agent over time that might or might not be at a constant level. Particularly, “controlled release” encompasses situations and formulations where the active ingredient is not necessarily released at a constant rate, but can include increasing release over time, decreasing release over time, and/or constant release with one or more periods of increased release, decreased release, or combinations thereof. Thus, while “sustained release” is a form of “controlled release”, the latter also includes delivery modalities that employ changes in the amount of an active agent (e.g., an SSCI composition) that are delivered at different times.

In some embodiments, the sustained release formulation, the controlled release formulation, or the combination thereof is selected from the group consisting of an oral formulation, a peroral formulation, a buccal formulation, an enteral formulation, a pulmonary formulation, a rectal formulation, a vaginal formulation, a nasal formulation, a lingual formulation, a sublingual formulation, an intravenous formulation, an intraarterial formulation, an intracardial formulation, an intramuscular formulation, an intraperitoneal formulation, a transdermal formulation, an intracranial formulation, an intracutaneous formulation, a subcutaneous formulation, an aerosolized formulation, an ocular formulation, an implantable formulation, a depot injection formulation, a transdermal formulation and combinations thereof. In some embodiments, the route of administration is selected from the group consisting of oral, peroral, buccal, enteral, pulmonary, rectal, vaginal, nasal, lingual, sublingual, intravenous, intraarterial, intracardial, intramuscular, intraperitoneal, transdermal, intracranial, intracutaneous, subcutaneous, ocular, via an implant, and via a depot injection. Where applicable, continuous infusion can enhance drug accumulation at a target site (see, e.g., U.S. Pat. No. 6,180,082). See also U.S. Pat. Nos. 3,598,122; 5,016,652; 5,935,975; 6,106,856; 6,162,459; 6,495,605; and 6,582,724; and U.S. Patent Application Publication No. 2006/0188558 for transdermal formulations and methods of delivery of compositions. In some embodiments, the administering is via a route selected from the group consisting of peroral, intravenous, intraperitoneal, inhalation, intraprostatic, and intratumoral.

The particular mode of drug administration used in accordance with the methods of the presently disclosed subject matter depends on various factors, including but not limited to the vector and/or drug carrier employed, the severity of the condition to be treated, and mechanisms for metabolism or removal of the drug following administration.

IV.D. Combination Therapies

In some embodiments, a method of treatment that comprises administration of an SSCI of the presently disclosed subject matter can also comprise any other therapy known or expected to be of benefit to a subject with a condition, disease, or disorder associated with undesirable COX (e.g., COX-2) biological activity. By way of example and not limitation, anxiety and pain are conditions, diseases, or disorders associated with an undesirable COX-2 biological activity. Any standard therapy that is used to treat anxiety and/or pain or a precursor condition thereof can be employed before, concurrently with, and/or after administration of a composition of the presently disclosed subject matter.

### V. METHODS OF DESIGNING AND/OR IDENTIFYING NEW SSCIS

The presently disclosed subject matter also provides methods for designing and/or identifying new SSCIs. In some embodiments, computer modeling software such as those disclosed herein are employed for designing SSCIs that interact with the COX-2 homodimer as do the SSCIs disclosed herein. For example, the (R)-profens are disclosed herein to

Alternatively or in addition, the presently disclosed subject matter provides for methods of identifying new SSCIs that employ libraries of compounds (e.g., a high-throughput screening library comprising potential SSCIs) that are tested for substrate-selective inhibitory activity. By way of example and not limitation, potential SSCIs can be incubated with a COX-2 enzyme preparation (e.g., a pure preparation or an isolate from a cell, tissue, or organ, or subject) in the presence of arachidonic acid and/or 2-arachidonoylglycerol or arachidonoylethanolamide. In some embodiments, the products are extracted and analyzed by liquid chromatography-mass spectrometry as described in herein to identify SSCIs.

### EXAMPLES

The following Examples provide further illustrative embodiments. In light of the present disclosure and the general level of skill in the art, those of skill will appreciate that the following Example is intended to be exemplary only and that numerous changes, modifications, and alterations can be employed without departing from the scope of the presently disclosed subject matter.

### Materials and Methods for EXAMPLES 1-5

Materials. Along with the wild type protein, mCOX-2 protein with the mutations R120Q, E524L, S530A, or Y355F were expressed in insect cells and purified as described previously in (Rowlinson et al., 1999). With respect to COX-2 amino acid mutations, the amino acid position numbers listed herein are based on the COX-1 sequence, which includes an additional 14 amino acids at the N-terminus. Hence, “R120Q” refers to a change from arginine to glutamine at the position that corresponds to R120 of COX-1. It is noted that this position actually corresponds to amino acid 106 in murine COX-2 as set forth in GENEBANK® database Accession No. NP_{—}035328 (which shows R106, E510, S516, and Y341, for example). Similarly, the gluamtic acid referred to herein as “E524” is actually at amino acid position 510 of mCOX-2, “Y335” is a tyrosine at amino acid position 321 of mCOX-2, etc. Human MAGL was purchased from Cayman Chemical. Humanized rat FAAH was a generous gift of R. Stevens and B. Cravatt (The Scripps Research Institute, La Jolla, Calif., United States of America). Human 15-lipoxygenase-1 was a generous gift of A. Brash (Vanderbilt University School of Medicine, Nashville, Tenn., United States of America).

### Inhibition of COX-2-Mediated Metabolism of Arachidonic Acid and 2-AG

Various concentrations of inhibitor (or DMSO) were incubated with mCOX-2 (200 nM) for 5-15 minutes in 100 mM Tris-HCl with 0.5 mM phenol, pH 8.0. For mutant mCOX-2 experiments, the enzyme concentration was adjusted such that the turnover was approximately equal to wild type enzyme. The preincubation time was determined on the basis of previous reports regarding the time required to achieve maximal inhibition, and preincubation was performed at 25° C. except for the final 3 minutes, which took place at 37° C. (Kalgutkar et al., 1998; Prusakiewicz et al., 2009). After the preincubation of enzyme and inhibitor, arachidonic acid or 2-AG was added for 30 seconds at 37° C. The reaction was quenched with ice-cold ethyl acetate containing 0.5% (v/v) acetic acid and 1 μM PGE₂-d₄ and PGE₂-G-d₅. The solution was then vigorously mixed and cooled on ice. The organic layer was separated and evaporated to near-dryness under nitrogen. For analysis, the samples were reconstituted in 1:1 MeOH:H₂O and chromatographed using a Luna C18(2) column (50 2 mm, 3 μm; Phenomenex Inc., Torrance, Calif., United States of America) with an isocratic elution method requiring 66% (v/v) 5 mM ammonium acetate, pH 3.3 (solvent A) and 34% (v/v) acetonitrile containing 6% (v/v) solvent A (solvent B) at a flow rate of 0.375 ml min⁻¹. MS/MS was conducted on a Quantum triple-quadrupole mass spectrometer operated in positive-ion mode using a selected reaction monitoring method with the following transitions: m/z 370→317 for PGE₂/D₂, m/z 374→321 for PGE₂-d₄, m/z 444→391 for PGE₂/D₂-G, and m/z 449→396 for PGE₂/D2-G-d₅. Peak areas for analytes were normalized to the appropriate internal standard to determine the amount of product formation, and the amount of inhibition was determined by normalization to a DMSO control.

### Crystallization, Data Collection, Structure Determination and Refinement

Protein crystallization was performed as described in (Duggan et al., 2010). Data sets were collected on an ADSC Quantum-315 CCD (Area Detector Systems Corp., Poway, Calif., United States of America) using the synchrotron radiation X-ray source tuned at a wavelength of 0.97929 Å with an operating temperature of 100 K at beamline 24ID-E of the Advance Photon Source at the Argonne National Laboratory (Lemont, Ill., United States of America). Diffraction data were processed with the software program HKL-2000 (HKL Research; Otwinowski & Minor, 1997). Initial phases were determined by molecular replacement using a search model (PDB 3NT1) with MOLREP (Vagin & Teplyakov, 2000). Solutions with two molecules in the asymmetric unit of (R)-naproxen and four molecules in that of (R)-flurbiprofen were obtained. The models were improved with iterative rounds of model building in Coot and with refinement in PHENIX (Adams et al., 2002; Emsley & Cowtan, 2004). Data collection and refinement statistics are reported in Table 2.

TABLE 2
Data Collection and Tefinement Statistics for the (R)-Naproxen-mCOX-
2 and (R)-Flurbiprofen-mCOX-2 Complexes

| Data Collection | (R)-Naproxen | (R)-Flurbiprofen |
|---|---|---|
| Space Group | I222 | P2₁2₁2 |
| Cell Dimensions | | |
| a, b, c, (Å) | 122.74, 133.03, 181.04 | 180.62, 134.55, 122.78 |
| α,β,γ (°) | 90.00, 90.00, 90.00 | 90.00, 90.00, 90.00 |
| R_{merge} (%) | 11.7 (43.4) | 11.8 (54.3) |
| I/σI | 15.7 (3.6) | 11.7 (3.3) |
| Completeness (%) | 99.9 (100.0) | 95.1 (92.2) |
| Redundancy | 5.7 (5.8) | 5.5 (5.3) |
| Refinement | | |
| Resolution (Å) | 2.4 | 2.85 |
| No. reflections | 55701 | 62160 |
| R_{work}/R_{free} (%) | 17.7/23.3 | 19.7/24.4 |
| No. of atoms | | |
| Protein | 8946 | 17896 |
| Ligand/ions | 322/2 | 588/0 |
| Water | 601 | 241 |
| B-factors | | |
| Protein | 2806 | 4708 |
| Ligand/ions | 42.0/33.0 | 59.4/0 |
| Water | 31.7 | 37.9 |
| Estimated | | |
| Coordinate | | |
| Error (Å) | | |
| Luzattoi plot | 0.25 | 0.35 |
| Maximum | 0.29 | 0.36 |
| likelihood | | |
| R.m.s. deviations | | |
| Bond lengths (Å) | 0.008 | 0.009 |
| Bond angles (°) | 1.3 | 1.2 |

Values in pararenthese correspond to the highest resulution shells.
Rmerge=Σₕₖ₁ Σ_{j=1,N}|<Iₕₖ₁>−Iₕₖₗⱼ|/Σₕₖₗ Σ_{j=1,N}|Iₕₖₗⱼ| where the outer sum (hkl) is taken over the unique reflections.
R_{work}=Σₕₖₗ∥F_{o,hkl}−k|F_{c,hkl}∥/Σₕₖₗ|F_{o,hkl}, where |F_{o,hkl}| and |F_{c,hkl}| are the observed and calculated structure factor amplitudes, respectively.
R_{free} is same as for R_{work} for the set of reflections (5% of the total) omitted from the refinement process.
NE-CAT, North Eastern Collaborative Access Team

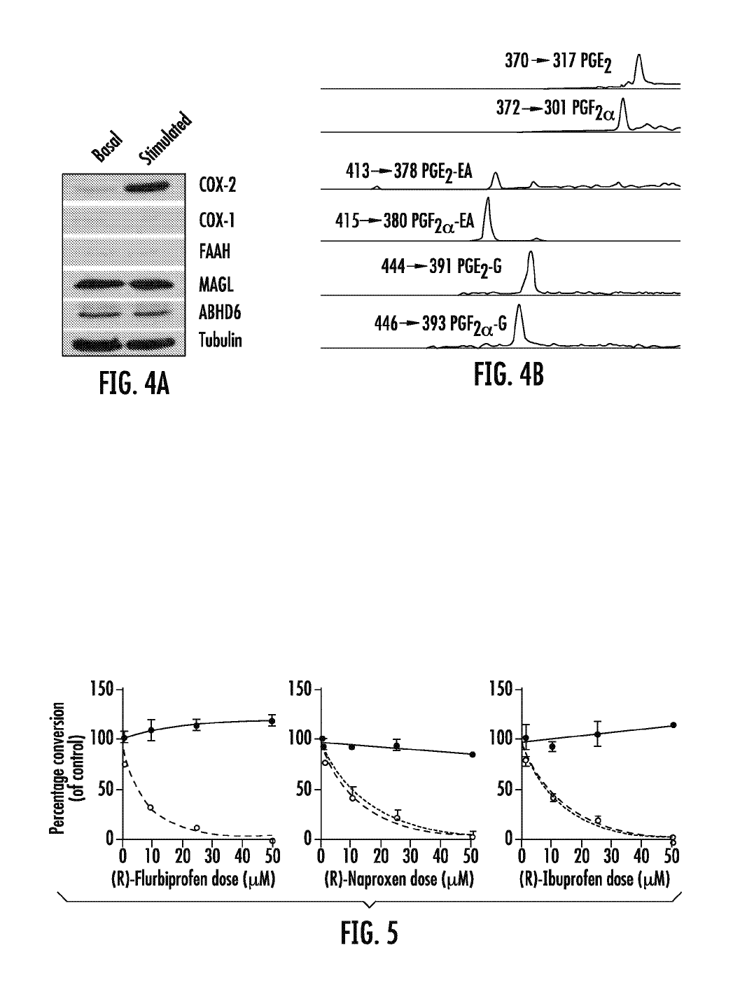

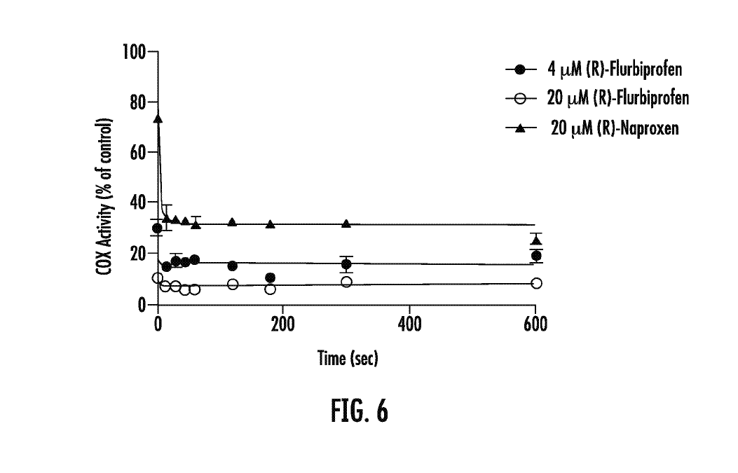

In the Ramachandran plot, 93.7% of all residues are in the most favored region for the (R)-naproxen/mCOX-2 structure, and 94.0% of all residues appear in the most favored region for the (R)-flurbiprofen/mCOX-2 structure. The estimated coordinate errors are 0.25 Å for (R)-naproxenlmCOX-2 and 0.29 Å for (R)-flurbiprofen/mCOX-2. Molecular graphics (see FIGS. 4 and 5) were illustrated with PyMOL (see the website of PyMOL, pymol[dot]org).

DRG Preparation.

DRG culturing and staining for neurons and glia was performed as described in Wu et al., 2009 using a protocol approved by the Vanderbilt University Institutional Animal Care and Use Committee. Staining for COX-2 was performed as described in Uddin et al., 2010. Treatment with inflammatory stimuli was performed as described herein below. Extraction and analysis of prostaglandins, PG-Gs, and PG-EAs was performed as described in Kingsley & Marnett, 2007.

Accession Codes.

Crystal structure coordinates were deposited with the RCSB Protein Data Bank (PDB) under the codes 3Q7D and 3RR3 for (R)-naproxen/mCOX-2 and for (R)-flurbiprofen/mCOX-2, respectively.

### Example 1

### Survey of COX Inhibitors for Substrate Selectivity

To explore the generality of substrate-selective inhibition, the effects of different classes of NSAIDs on the COX-2-dependent oxygenation of arachidonic acid and 2-AG were compared. An assay that measured prostaglandin or PG-G formation by LC-MS/MS following a 15 minute preincubation of enzyme and inhibitor to ensure maximal inhibition of the oxygenation of both substrates was employed. Saturating concentrations of both arachidonic acid and 2-AG (50 μM) were used.

The results are summarized in Table 3. Weak, reversible inhibitors of arachidonic acid oxygenation were strong inhibitors of 2-AG oxygenation, whereas slow, tight-binding inhibitors were potent inhibitors of both 2-AG and arachidonic acid oxidation by COX-2, with comparable half-maximum inhibitory concentration (IC₅₀) values for both substrates (see Table 3). While not wishing to be bound by any particular theory of operation, the ability of slow, tight binding inhibitors to inhibit 2-AG and arachidonic acid oxygenation at comparable concentrations might arise from the capacity of a single molecule of inhibitor to block the oxygenation of both substrates (Kulmacz & Lands, 1985; Prusakiewicz et al., 2009; Rimon, G. et al., 20101 Dong et al., 2011). Within inhibitor classes, no differences in behavior were observed between compounds that were COX-2-selective inhibitors or nonselective inhibitors of both COX enzymes (for example, celecoxib versus diclofenac).

TABLE 3
IC₅₀ Values for the Inhibition of COX-2 Oxygenation of
Arachidonic Acid and 2-AG by NSAIDs

| | Inhibitor | 50 μM AA | 50 μM 2-AG |
|---|---|---|---|
| Reversible | Ibuprofen^{b,c} | 180.0 μM | 210 nM |
| | Mefanamic acid^{b.c} | 7.0 μM | 20 nM |
| | DM-INDO | >25.0 μM | 250 nM |
| | Lumiracoxib | No inhibition | 40 nM |
| | Maproxen^{b} | 4.5 μM | 430 nM |
| | SC-58076 | >4.0 μM | 40 nM |
| Slow, tight binders | Diclofenac | 60 nM | 50 nM |
| | Fluribiprofen | 130 nM | 30 nM |
| | INDO | 180 nM | 30 nM |
| | Celecoxib | 80 nM | 95 nM |
| | Rofecoxib | 520 n> | 85 nM |

^{a}Enzyme and inhibitor were preincubated for 15 minutes before the addition of 50 μM substrate for 30 seconds. Reactions were quenched with organic solvent containing deuterated internal standards. Product formation was analyzed by LC-MS/MS using selected reaction monitoring and was notmalized to DMSO control values.
^{b}Substrate oxygenation was measured using an oxygen electrode.
^{c}Values for ibuprofen and mefenamic acid taken from Prusakiewicz et al., 2009.

### Example 2

### Inhibition by (R)-Profens

Profens showed marked enantiospecificity for inhibition of arachidonic acid oxygenation by COX enzymes. The (S) enantiomers of naproxen, ibuprofen, and flurbiprofen inhibited arachidonic acid oxygenation by COX-1 and COX-2, but the (R) enantiomers inhibited either poorly or not at all.

The ability of the (R) enantiomers of naproxen, ibuprofen, and flurbiprofen to inhibit 2-AG oxygenation by COX-2 was examined. (R)-flurbiprofen (4 or 20 μM) or (R)-naproxen (20 rM) were incubated with mCOX-2 for 0, 15, 30, 45, 60, 120, 180, 300, or 600 seconds prior to the addition of a saturating concentration of 2-AG for 30 seconds. Reactions were quenched and PG-G's were measured by LC-MS/MS as described herein above under the heading “Materials and Methods for the EXAMPLES”.

The results are shown in FIG. 6 and in Table 4 herein below. As set forth therein, the (R) enantiomers inhibited 2-AG oxidation. When concentrations of 2-AG were nearer its Km (5 μM) than its saturation point (50 μM), the inhibitors were more potent. Notably, (R)-naproxen, (R)-ibuprofen, and (R)-flurbiprofen did not inhibit arachidonic acid oxidation by COX-2 at either 5 μM or 50 μM substrate concentrations.

TABLE 4
(R)-Profen Inhibition of 2-AG Oxygenation by COX-2^{a}

| Inhibitor | 50 μM 2-AG | 5 μM 2-AG |
|---|---|---|
| 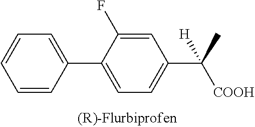 [SMILES: C[C@@H](C(=O)O)c1ccc(-c2ccccc2)c(F)c1] | 3.7 μM | 0.08 μM |
| 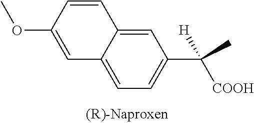 [SMILES: COc1ccc2cc([C@@H](C)C(=O)O)ccc2c1] | 6.7 μM | 3 μM |
| 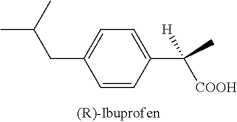 | 18 μM | 10 μM |

^{a}Enzyme and inhibitor were preincubated for 15 min before the addition of substrate for 30 s. Reactions were quenched with organic solvent containing deuterated internal standards. Product formation was analyzed by LC-MS/MS using selected reaction monitoring and was normalized to DMSO control.

### Example 3

### Structure-Function Analysis of (R)-Profen Inhibition

The findings disclosed herein illustrated that the (R) enantiomers of arylpropionic acids bound to COX-2 and inhibited the oxygenation of 2-AG. This was surprising given prior results suggesting that predicted steric clashes with active site residues would be expected to prevent the binding of (R)-arylpropionic acids within the COX active site (Bhattacharyya et al., 1996). Therefore, crystallized complexes of each of the (R) enantiomers bound to mCOX-2 were produced and analyzed to identify their binding sites.

Diffraction-quality crystals were obtained with both (R)-naproxen and (R)-flurbiprofen using a methodology described in (Duggan et al., 2010). An experimental electron density map for (R)-naproxen bound to murine COX-2 (mCOX-2) was produced with a simulated annealing omit map (Fₒ-F_{c}) contoured at 30. The inhibitor was located exclusively within the cyclooxygenase active site; its carboxylate moiety was adjacent to Arg120 and Tyr355 at the mouth of the active site, and its naphthyl ring projected up into the center of the cyclooxygenase channel. This is typical of the orientation of arylpropionic acids in the COX active site as has been reported for (S)-ibuprofen complexed to COX-1, (S)-naproxen complexed to COX-2, and (S)-flurbiprofen complexed to COX-1 or COX-2 (Kurumbail et al., 1996; Selinsky et al., 2001; Duggan et al., 2010).

Previous reports based on site-directed mutagenesis and structure-activity studies had suggested that stable binding of (R)-arylpropionic acids within the active site of COX would be prevented by unfavorable steric interactions between the α-methyl group and Tyr355 at the base of the active site (see Bhattacharyya et al., 1996). Surprisingly, the α-methyl group of (R)-naproxen bound adjacent to Tyr355.

The (S)-naproxen structure was recently reported at 1.7 Å, and an overlay of (R)-naproxen and (S)-naproxen bound in the active site of mCOX-2 was generated (see Duggan et al., 2010). The p-methoxy substituents and the two naphthyl rings were nearly superimposed, and the two enantiomers seemed to participate in many of the same interactions with surrounding protein residues. These included hydrogen bonding with Arg120 and Tyr355 as well as hydrophobic interactions with Ala527, Va1349, Gly526, Trp387, Tyr385, and Leu352.

However, (R)-naproxen did appear to participate in some interactions distinct from those of (S)-naproxen. The α-methyl group of (R)-naproxen participated in van der Waals interactions with Ser530 and Ser353, but the α-methyl substituent of (S)-naproxen did not. The principal difference in protein structure between the two complexes was the repositioning of Arg120 and Tyr355 to accommodate the α-methyl group in the binding of (R)-naproxen (r.m.s. deviations of 0.47 Å and 0.45 Å, respectively). This increase in the hydrogen-bond distance between Tyr355 and the carboxylate of (R)-naproxen to 3.05 Å, which was much further compared to the distance of 2.44 Å for (S)-naproxen, could reduce the binding energy of the (R)-naproxen/COX-2 complex.

Like the naproxen enantiomers, the (R) and (S) enantiomers of flurbiprofen bound in a similar fashion within the mCOX-2 active site (see FIG. 7). (R)-Flurbiprofen interacted with Arg120 and Tyr355 at the base of the active site and with Ala527, Va1349, Gly526, Tyr385, Leu359, and Ser530 in the hydrophobic channel.

To probe the importance of individual residues in the inhibition of 2-AG oxygenation, the inhibitory activity of (R)-flurbiprofen against a series of active site mutants was measured. The R120Q mutation, which eliminated the ability of that residue to participate in ion-pairing interactions, abolished (R)-flurbiprofen inhibition (see FIG. 7). In contrast, the Y355F, E524L, or S530A mutations did not have measurable effects on the IC₅₀ values of (R)-flurbiprofen compared to those of wild type mCOX-2 (see FIG. 7). These data were consistent with the binding mode of (R)-flurbiprofen observed in the crystal structure and suggested that ion pairing between the carboxylate and Arg120 was a critical determinant of binding.

### Example 4

### COX-2 Action in DRGs

(R)-Flurbiprofen has analgesic activity in humans and inhibits neuropathic pain in rodents (see Lo\ddot{t}sch et al., 1995; Bishay et al., 2010). It is inefficiently converted to (S)-flurbiprofen in vivo and does not show gastrointestinal toxicity, which is typically observed with compounds that inhibit COX-dependent prostaglandin synthesis (see Jamali et al., 1988; Brune et al., 1992). Notably, (R)-flurbiprofen has been reported to elevate AEA in the dorsal horn of rats surgically treated to induce nerve injury (see Bishay et al., 2010). The mode of action by which (R)-flurbiprofen causes analgesia and AEA elevation is uncertain, although its weak inhibition of FAAH (IC₅₀>1 mM in vitro) has been suggested as a possible explanation (see Bishay et al., 2010).

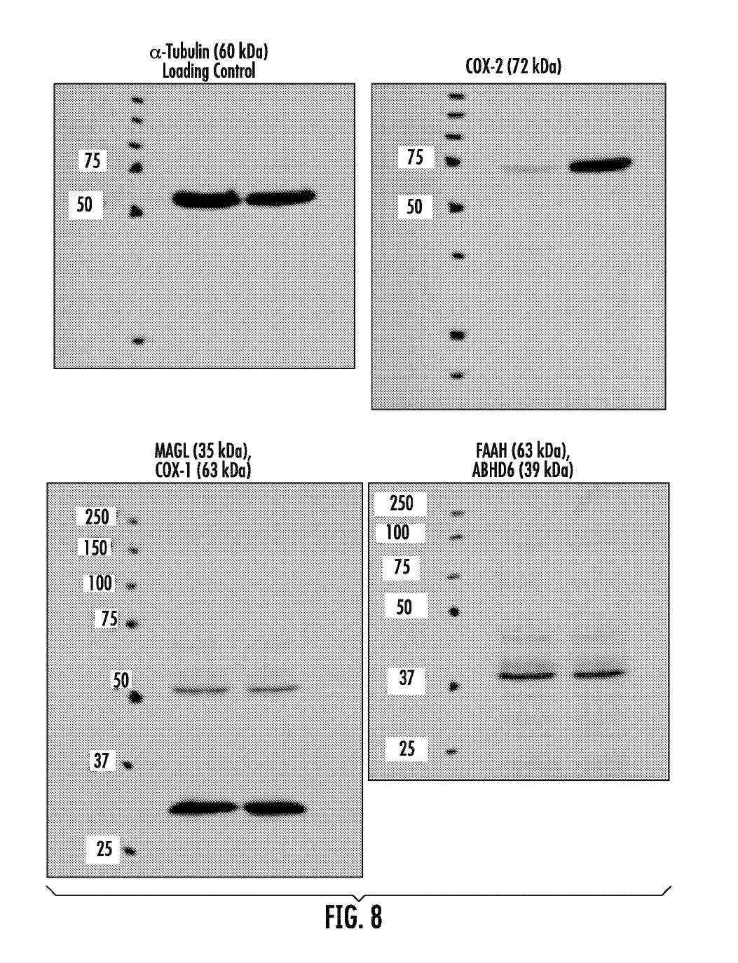

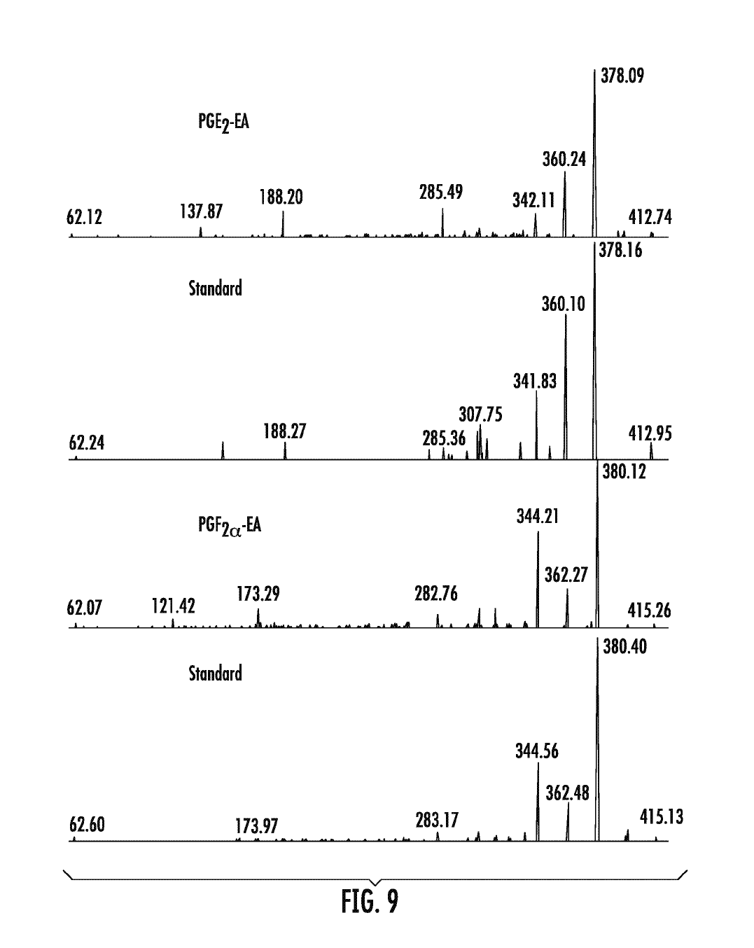

The analgesic activity of (R)-flurbiprofen could also be explained by its inhibition of the COX-2-selective metabolism of endocannabinoids. To evaluate this possibility and to test whether substrate-selective inhibition can be detected in intact cells stimulated to release physiological amounts of 2-AG and AEA, cellular experiments were performed with DRGs. DRGs were harvested from E14 mouse embryos and plated onto collagen-coated dishes. After being cultured for 3-5 days, they were treated overnight with granulocyte-macrophage colony-stimulating factor followed by lipopolysaccharide, interferon γ, and 10 μM 15(S)-hydroxy-5,8,11,13-eicosatetraenoic acid for 6 hours. This resulted in a strong induction of COX-2 but not COX-1 in the DRGs and no increase in the amounts of MAGL, ABHD6, or FAAH (see FIG. 8). The presence of COX-2 in the DRGs, where it was located mainly in neuronal cell bodies, was verified by the uptake of a COX-2-selective fluorescent imaging agent. The design and synthesis of this compound, fluorocoxib A, was recently described along with studies validating its selective binding to COX-2 in cultured cells in vitro and inflammatory lesions and tumors in vivo (see Uddin et al., 2010).

DRGs, activated as above for 3 hours were treated with ionomycin for an additional 3 hours to stimulate substrate release. The substrates and products of COX-2-mediated oxygenation were extracted and identified by LC-MS/MS. Peaks were detected that co-eluted with prostaglandin, PG-G, and PG-EA standards. In all cases, the major products were PGF_{2α} and PGE₂, their glyceryl esters, and ethanolamide derivatives.

It is noteworthy that the stimulation of DRGs resulted in the generation of PG-EAs. This was the first time that these oxygenated metabolites of AEA had been detected in intact cells stimulated to release endogenous COX-2 substrates. The identity of the PG-EAs was verified by collision-induced dissociation and analysis of the fragment ions (see FIG. 9). Thus, DRGs released arachidonic acid, 2-AG, and AEA and oxygenated them to form PGF_{2α} and PGE₂ derivatives following stimulation with proinflammatory mediators.

### Example 5

### Substrate-Selective Inhibition in DRGs

The amounts of prostaglandin, PG-G, and PG-EA were quantified using stable-isotope dilution methods with labeled internal standards. The addition of increasing concentrations of (R)-flurbiprofen, (R)-ibuprofen, or (R)-naproxen 1 hour before the addition of ionomycin inhibited the synthesis of endocannabinoid-derived eicosanoids, PG-Gs, and PG-EAs at concentrations that did not inhibit the synthesis of prostaglandins. The concentration-dependence values for inhibition of 2-AG oxygenation and AEA oxygenation were similar (see FIG. 5).

Thus, the substrate-selective inhibition of endocannabinoid oxygenation observed with purified COX-2 was also observed in intact DRGs stimulated with physiological agonists and endogenous substrates. The IC₅₀ values for 2-AG and AEA oxidation in DRGs by the (R)-profens ((R)-flurbiprofen-2-AG, 5.8±2.9 μM, (R)-flurbiprofen-AEA, 6.0±2.7 μM; (R)-naproxen-2-AG, 8.9±3.2 μM, (R)-naproxen-AEA, 11.8±4.1 μM; (R)-ibuprofen-2-AG, 10.1±4.7 μM, (R)-ibuprofen-AEA, 9.4 Å-4.3 μM) were closer to those for pure COX-2 at 50 μM 2-AG than at 5 μM 2-AG (see Table 4 above). A closer correspondence to values at low substrate concentrations in vitro might have been expected, but there were many differences between the in vitro and cellular assays, including different incubation times (30 seconds versus 4 hours), the presence of serum in the cell culture medium, and the presence of multiple competing fatty acids and other lipids in the activated DRGs. These might have had the effect of increasing the IC₅₀ values of the (R)-profens in intact DRGs relative to those in purified enzyme.

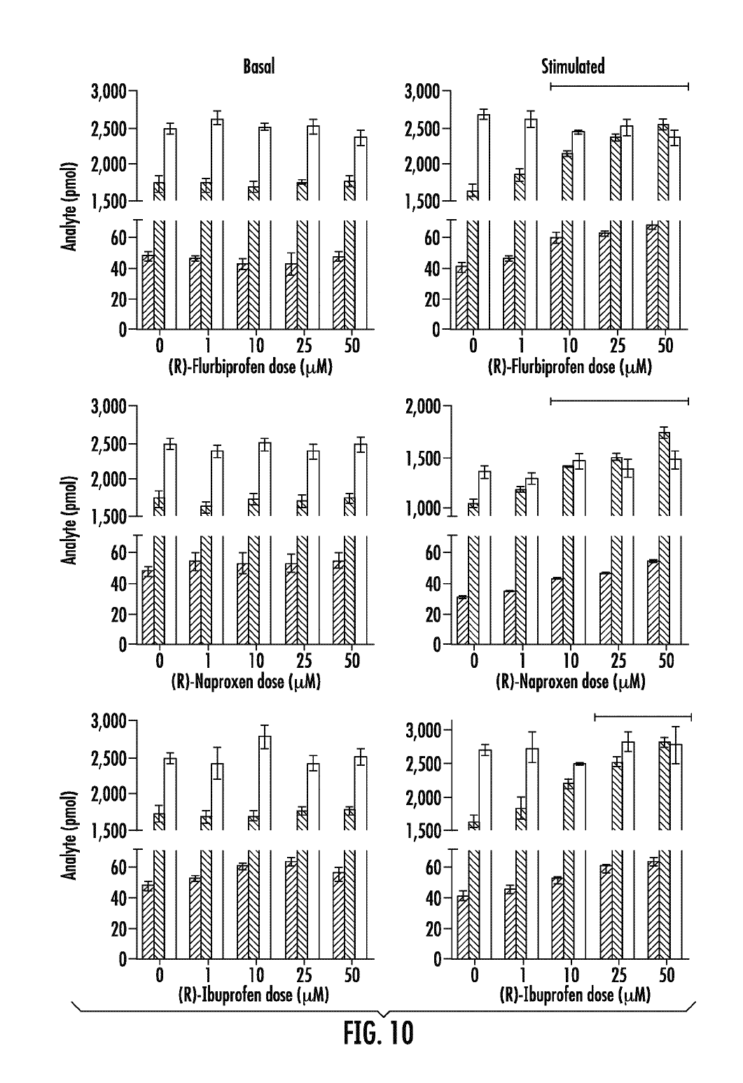

Concomitant with the inhibition of PG-G and PG-EA formation, treatment of stimulated DRGs with (R)-flurbiprofen, (R)-ibuprofen, and (R)-naproxen increased the amounts of AEA and 2-AG measured in cell extracts but did not increase the amounts of arachidonic acid (see FIG. 10). Notably, treatment of DRGs that were not stimulated with proinflammatory agonists did not increase the concentrations of 2-AG or AEA, which suggested that they did not inhibit the catalytic activity of MAGL or the monoacylglycerol lipase ABHD6, which appeared to be present in the cells.

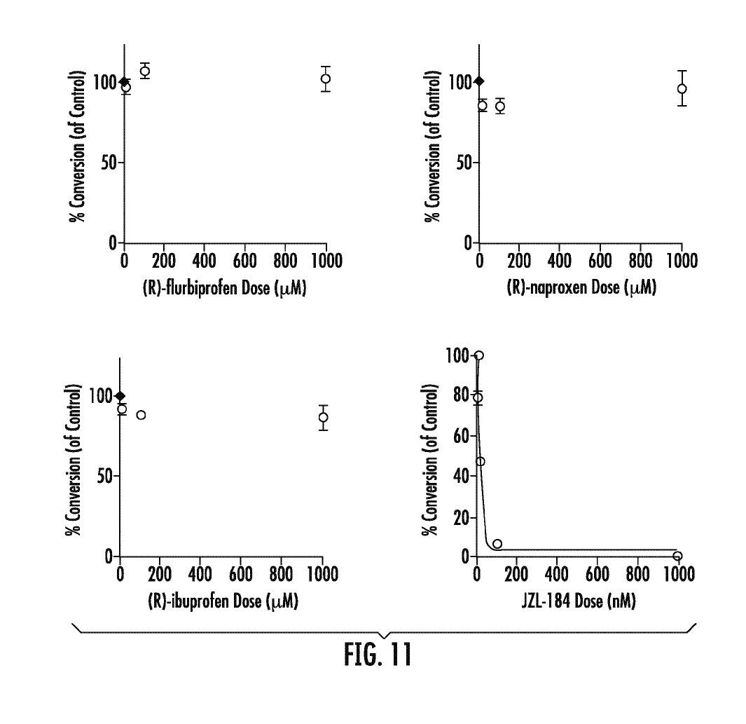

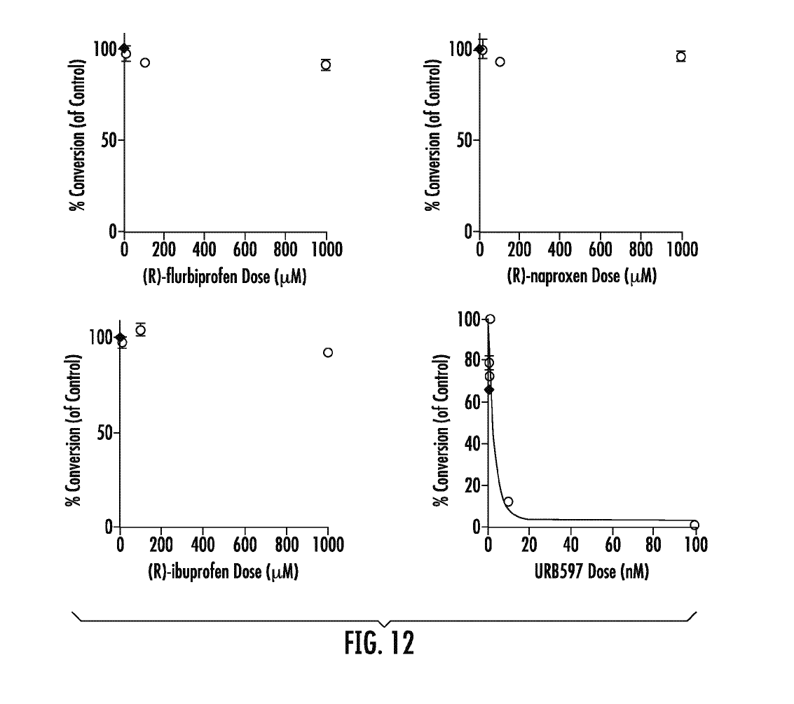

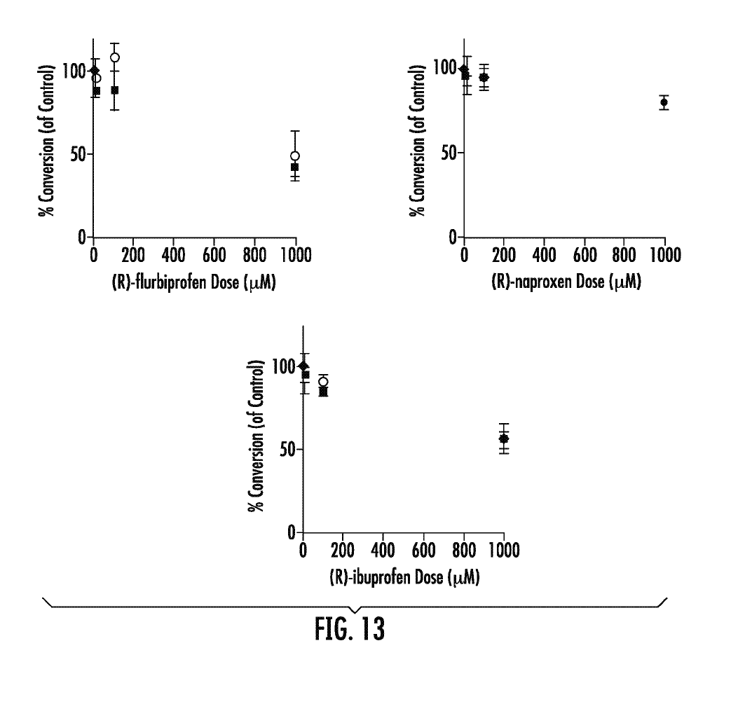

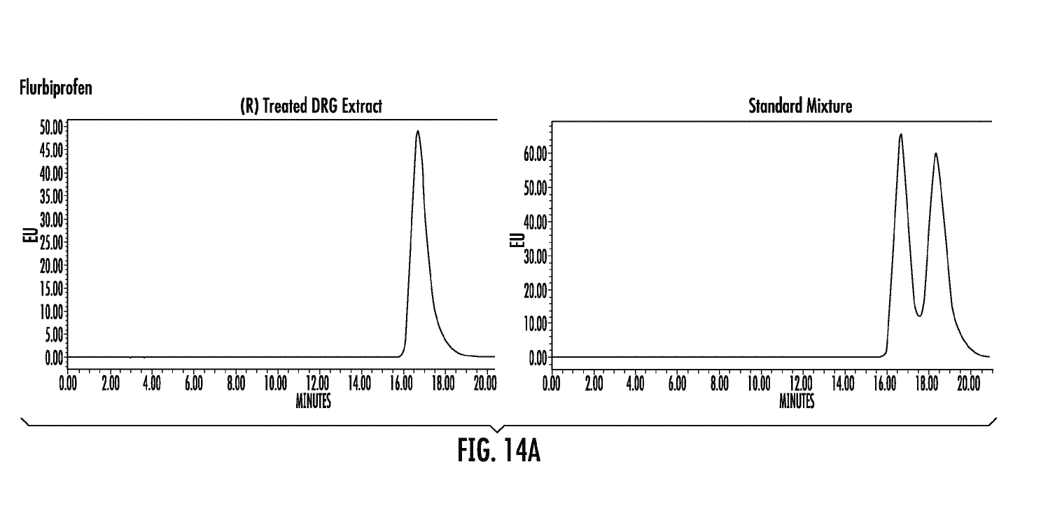

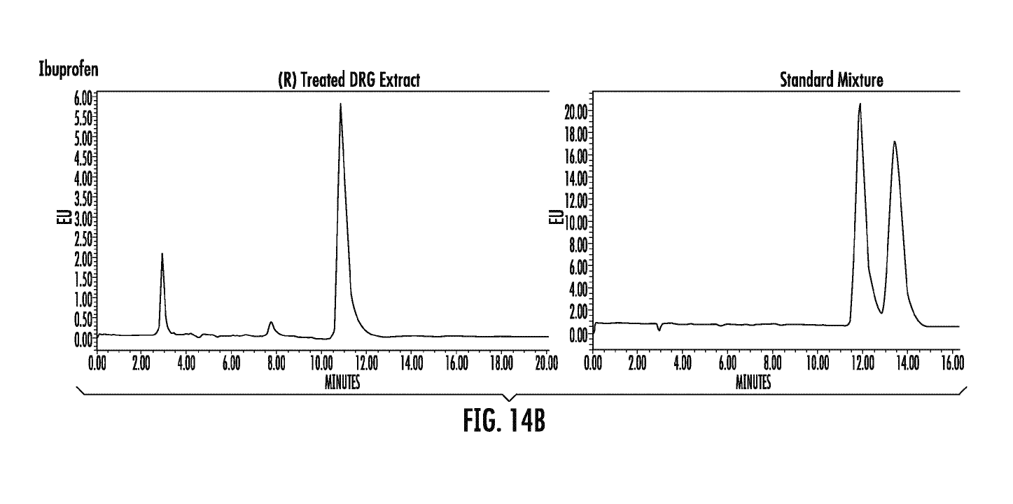

Notably, FAAH did not seem to be present in the DRGs. Indeed, incubation of increasing concentrations of the three (R)-profens with purified MAGL or FAAH in vitro caused no inhibition of their catalytic activities at concentrations up to 1 mM (see FIGS. 11 and 12). Similar control experiments indicated that (R)-profens did not inhibit 15-lipoxygenase-1 oxygenation of arachidonic acid or 2-AG at concentrations used in DRGs (see FIG. 13).

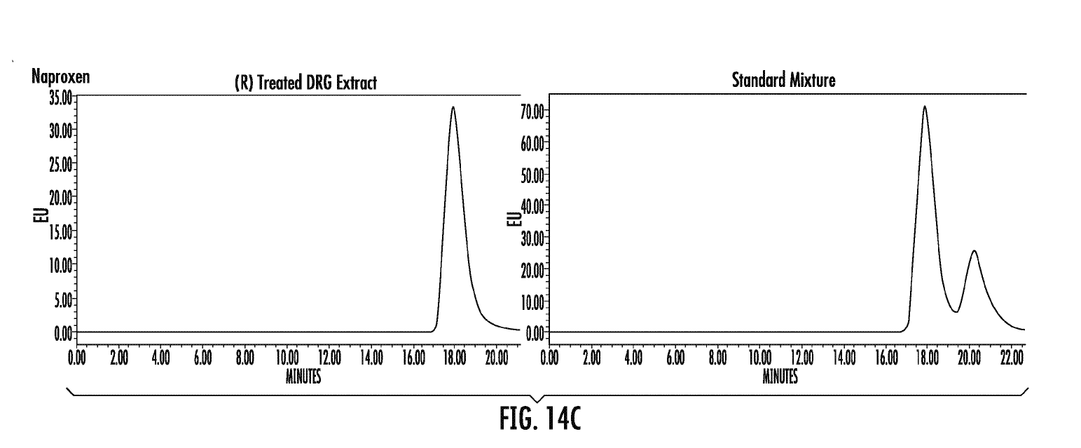

Finally, the enantiomeric composition of the (R)-profens that were recovered after a 4 hour incubation with DRGs was evaluated by chiral chromatography and was shown to be >99% (R) (see FIG. 14). Thus, no racemization occurred during incubation with the cells, leading to the conclusion that the substrate-selective inhibition of endocannabinoid oxygenation observed with the various profens was due to the (R) enantiomers.

### Discussion of EXAMPLES 1-5

The results presented herein above expand the range of compounds capable of substrate-selective inhibition of endocannabinoid oxygenation and indicated that such inhibition was limited to compounds characterized as rapid, reversible inhibitors of COX-2. The results also illustrated that (R) enantiomers of arylpropionic acid inhibitors, which were previously not considered COX inhibitors because of their inability to inhibit arachidonic acid oxygenation, actually bound to the enzyme and potently inhibited endocannabinoid oxygenation. Crystallographic studies indicated that the binding sites of (R)-naproxen and (R)-flurbiprofen were exclusively within the COX-2 active site, and functional studies indicated that ion pairing to Arg120 was critical for (R)-flurbiprofen binding.

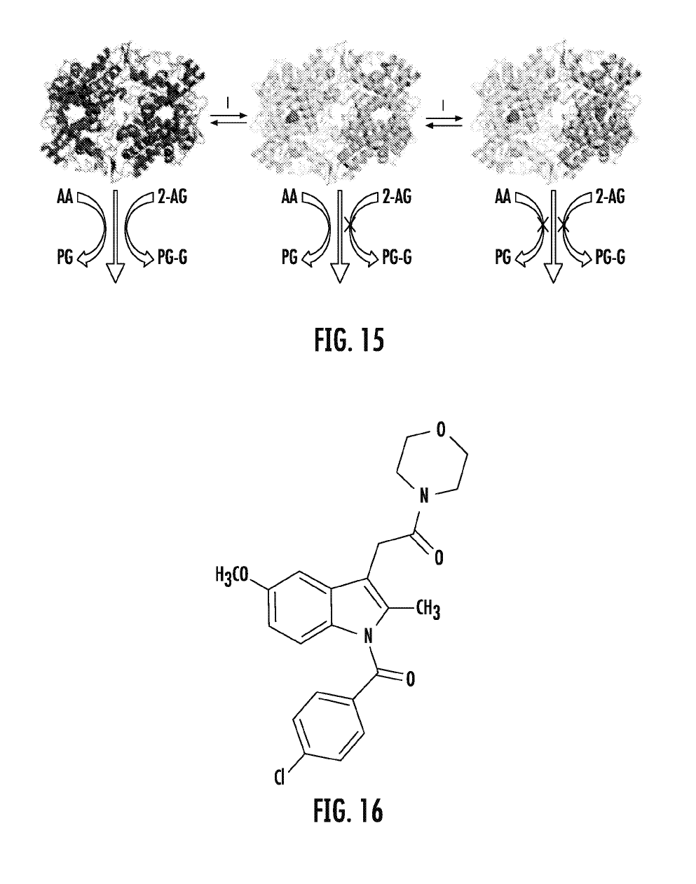

The potency of (R)-profen inhibition of endocannabinoid oxygenation dramatically illustrates the negative cooperativity between the two subunits of the COX-2 homodimer that results from binding of an inhibitor molecule to a single subunit (see FIG. 15; see also Prusakiewicz et al., 2009; Rimon et al., 2010; Dong et al., 2011). Although COX-2 and COX-1 are structural homodimers, they behave as functional heterodimers with an allosteric site and a catalytic site (see Dong et al., 2011). Kinetic analysis of substrate-selective inhibition of 2-AG oxygenation by (S)-ibuprofen suggested that it is a noncompetitive inhibitor that binds in the allosteric site (see Prusakiewicz et al., 2009). Thus, binding of (R)-profens likely occured in the allosteric site and induced a conformational change that prevented oxygenation of endocannabinoids, but not arachidonic acid, in the catalytic site (see FIG. 15).

COX-2 oxygenation of 2-AG and AEA in intact cells produces PG-G and PG-EA derivatives that have a range of biological activities (see Nirodi et al., 2004; Rouzer & Marnett, 2005; Yang & Chen, 2008; Woodward et al., 2008; Richie-Jannetta et al., 2010). The receptors responsible for these effects have not been identified, but they seem to be distinct from classic prostaglandin receptors (see Nirodi et al., 2004; Liang, et al., 2008). Thus, COX-2-dependent endocannabinoid oxygenation might represent a new mechanism for generating lipid signaling molecules dependent on different sets of agonists and phospholipases that are responsible for COX-2- (or COX-1-) dependent prostaglandin formation.

Testing this hypothesis in cellular systems or animal models has been difficult because of the lack of specific reagents that can differentiate COX-2-dependent 2-AG and AEA oxygenation from arachidonic acid oxygenation. The high degree of substrate-selective inhibition shown by (R)-profens suggested that they could be valuable probes for dissecting the specific contributions of COX-2 to cellular physiology or pathophysiology of endocannabinoid oxygenation from those of arachidonic acid oxygenation.

The metabolism of endocannabinoids and their relationship to signaling involves a complex set of enzymes and receptors (see Piomelli; 2003; Di Marzo, 2009). Following their biosyntheses from phospholipid precursors, AEA and 2-AG bind, respectively, to the CB1 receptor and TRPV1 and to the CB1 and CB2 receptors to stimulate cellular responses. The concentrations of AEA and 2-AG are primarily controlled through hydrolysis by FAAH and MAGL, respectively, although other enzymes will also hydrolyze 2-AG (for example, ABHD6, ABHD12 and carboxyl esterase 1; see Blankman et al., 2007; Xie et al., 2010). AEA and 2-AG are also oxygenated by COX-2, lipoxygenases, and cytochromes in the P450 family, and it is conceivable that, under certain conditions, oxygenation sufficient to further lower endocannabinoid concentrations could occur.

COX-2 is a particularly attractive candidate for modulation of endocannabinoid concentrations because it is highly induced by a range of agents, including proinflammatory stimuli. COX-2 production was induced in DRGs stimulated with proinflammatory agents; in contrast, the amounts of MAGL, FAAH, and ABHD6 were not increased (see FIG. 4). Stimulation with proinflammatory agents resulted in substantial COX-2-mediated oxygenation of arachidonic acid, 2-AG, and AEA, but without pretreatment with proinflammatory stimuli, no oxygenation was observed. Incubation of DRGs with (R)-profens selectively inhibited oxygenation of 2-AG and AEA but not oxygenation of arachidonic acid (see FIG. 5). (R)-Profens also increased the amounts of 2-AG and AEA but not the amount of arachidonic acid (see FIG. 10). Interestingly, (R)-profens did not increase 2-AG and AEA in DRGs that were not pretreated with proinflammatory stimuli (see FIG. 10). This observation was consistent with COX-2 reducing endocannabinoid concentrations by oxygenation of PG-Gs and PG-EAs selectively inhibiting their oxygenation.

These findings uncover a potential mechanism for the analgesic activity of (R)-flurbiprofen. The ability of (R)-flurbiprofen to selectively inhibit AEA and 2-AG oxygenation in DRGs correlates to its ability to elevate AEA concentrations at sites of neuroinflammation in the spinal cord (see Bishay et al., 2010). Although FAAH and MAGL are most likely responsible for the basal turnover of endocannabinoids in non-inflamed tissue, diurnal fluctuations lead to increases in COX-2 in regions of the brain, and induction of inflammation in the peripheral or central nervous system by nerve injury results in elevations of COX-2 concentrations in the inflamed tissue (see Guay et al., 2004; Glaser & Kaczocha, 2010). COX-2 induction might contribute to the depletion of AEA and 2-AG, and blockage of this depletion by substrate-selective inhibition of COX-2 by (R)-flurbiprofen could spare endocannabinoid concentrations and induce analgesia. Consistent with this mechanism, the analgesic effect of (R)-flurbiprofen is prevented by CB1-receptor antagonists despite the fact that (R)-flurbiprofen does not activate the CB 1 receptor (see Bishay et al., 2010). This highlights the importance of maintaining endocannabinoid tone in the analgesic action of (R)-flurbiprofen.

### Example 6

### Development of a Substrate-Selective Inhibitor of COX-2

Described herein above are a series of experiments that demonstrated that rapid-reversible inhibitors of cyclooxygenase-2 (COX-2) are substrate-selective inhibitors, while slow, tight-binding inhibitors of COX-2 are non-substrate-selective inhibitors. In particular, it was determined that the (R)-profens, which had previously been classified as inactive toward COX-2, are actually substrate-selective inhibitors of COX-2. It was also determined that (R)-profens increased the levels of anandamide (AEA) and 2-arachidonoyl glycerol (2-AG) while decreasing the levels of prostaglandin ethanolamides (PG-EAs) and prostaglandin glycerol esters (PG-Gs) in primary murine dorsal root ganglia cells stimulated to express COX-2. In vivo, however, (R)-profens undergo a one way stereoisomerization to (S)-profens, which are non-substrate-selective inhibitors of COX-2.

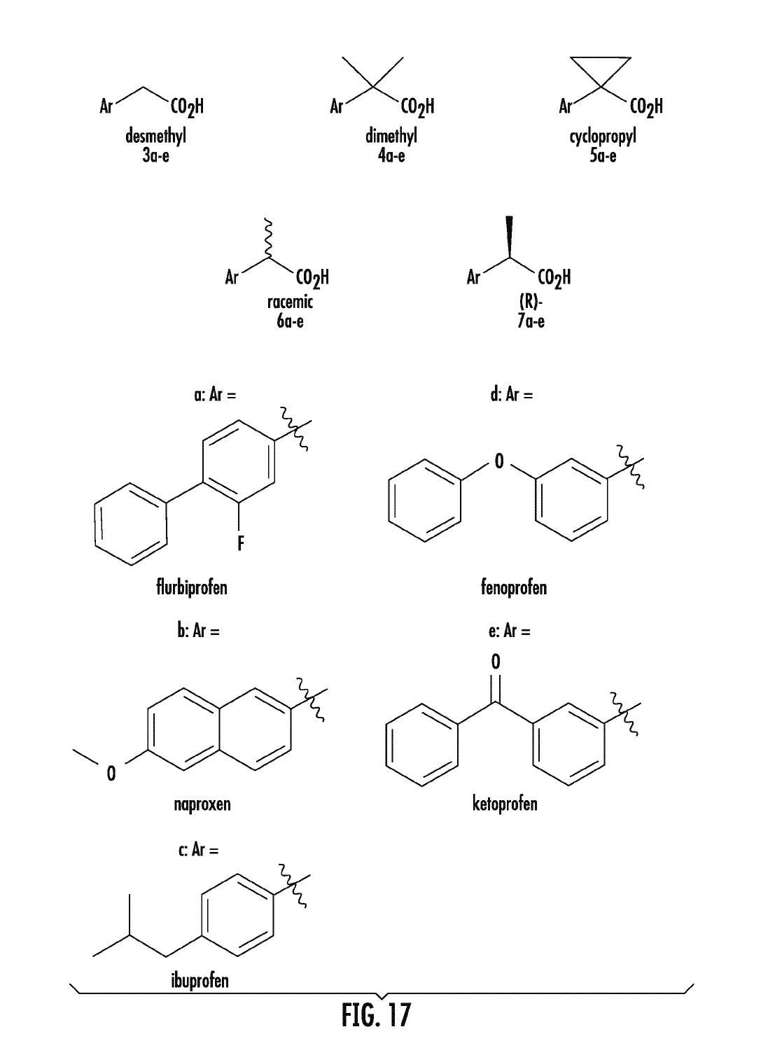

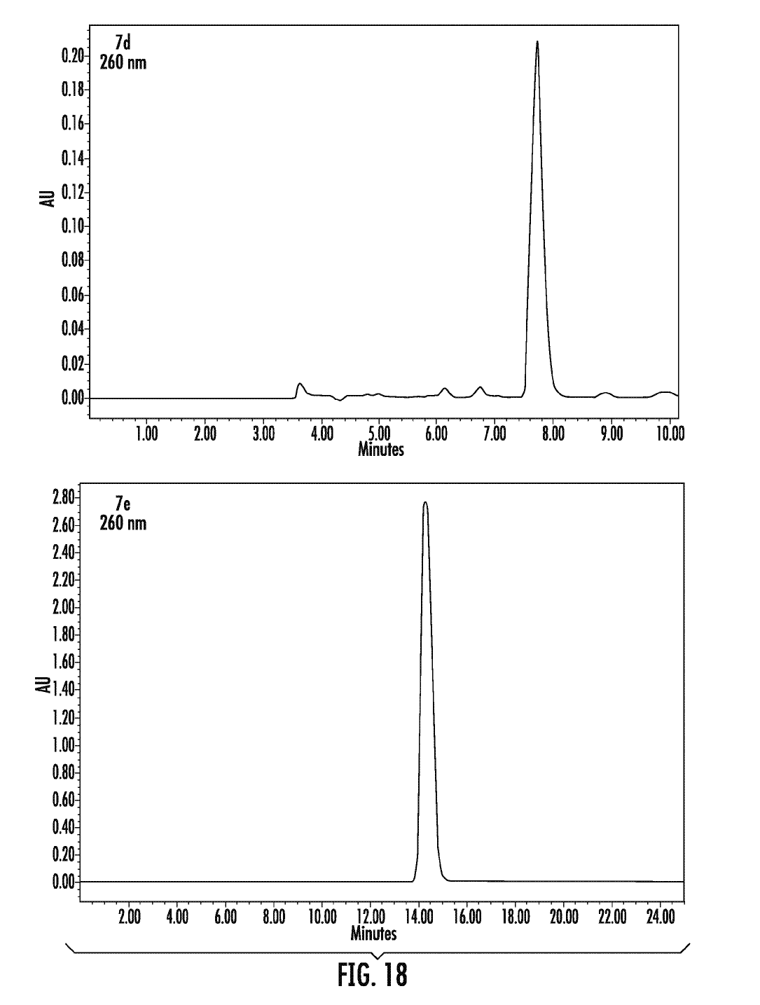

Disclosed herein are additional SSCIs that in some embodiments can be employed in vivo. Through the use of site-directed mutagenesis it was determined that disruption of the hydrogen bonding and ion pairing network can cause slow, tight-binding, non-substrate-selective inhibitors to become rapid-reversible, substrate-selective inhibitors. Based on the site-directed mutagenesis data, it was determined that tertiary amides of indomethacin are potent substrate-selective inhibitors of COX-2. In particular, the morpholino amide of indomethacin, 2-[1-(4-Chloro-benzoyl)-5-methoxy-2-methyl-1H-indol-3-yl]-1-morpholin-4-yl-ethanone (referred to herein as “Compound A”; see FIG. 16), was a potent substrate-selective inhibitor with an IC₅₀ of 620 nM for inhibition of 2-AG oxygenation by COX-2. Compound A also inhibited PG-G production in stimulated RAW 264.7 macrophages with an IC₅₀ of 660 nM while increasing the levels of 2-AG. It was also determined that Compound A did not inhibit fatty acid amide hydrolase (FAAH) in vitro.

### Example 7

### Acetaminophen and its Metabolites, 4-Aminophenol and AM-404, are SSCIs

Cyclooxygenase-2 (COX-2) catalyzes the oxygenation of arachidonic acid (AA) and the endocannabinoids, 2-arachidonoylglycerol (2-AG) and arachidonoylethanolamide (AEA), to prostaglandin endoperoxide derivatives. Non-steroidal anti-inflammatory drugs such as ibuprofen, naproxen, and lumiracoxib, which are weak, competitive inhibitors of AA oxygenation, are potent non-competitive inhibitors of 2-AG and AEA oxygenation. These compounds are as much as 1,000-fold more potent at inhibiting endocannabinoid oxygenation than AA oxygenation. The instant co-inventors have termed this phenomenon “substrate-selective inhibition” (SSI-COX-2) and propose that it contributes to the pharmacological effects of certain NSAIDs by preventing oxidative endocannabinoid metabolism at sites of COX-2 induction.

The analgesic agent, acetaminophen (APAP), and its in vivo metabolites 4-aminophenol and N-(4-hydroxyphenyl)arachidonoylamide (AM-404), exhibit SSCI activity with IC₅₀'s of 233 μM, 71 μM, and 97 nM, respectively, for inhibition of 2-AG oxygenation. No inhibition of AA oxygenation was observed up to 1 mM for APAP or 4-aminophenol. AM-404 exhibited 700-fold selectivity for inhibition of 2-AG and AEA oxygenation compared to AA oxygenation.

APAP and AM404 selectively inhibited 2-AG oxygenation by lipopolysaccharide-activated RAW264.7 cells with IC₅₀'s of 97 μM and 46 nM, respectively. Structure-activity analysis of a series of AM404 analogs revealed that the presence of a hydroxyl group at the para-position of the phenylamide ring was important for SSI-COX-2. Dissociation of AM404 from COX-2 was slow in the absence of substrate but was rapid in the presence of AA. Inhibition of endocannabinoid oxygenation was reversed by addition of nanomolar concentrations of AA. The observations were consistent with a model for SSCIs in which APAP, 4-aminophenol, or AM404 binds to the allosteric subunit of COX-2 to inhibit endocannabinoid oxygenation in the catalytic subunit. Substrate-selective inhibition of endocannabinoid oxygenation might therefore contribute to the pharmacological actions of APAP and its metabolites.

### Example 8

### Substrate Selective Inhibition of COX-2 as a Novel Strategy for In Vivo Endocannabinoid Augmentation

Development of endocannabinoid (eCB) degradation inhibitors has significantly advanced the therapeutic potential of eCB signaling for a variety of pathological conditions including mood and anxiety disorders. COX-2 degrades both AEA and 2-AG, and activation of eCB signaling contributes to the analgesic effects of COX-2 inhibitors. However, therapeutic development of COX-2 inhibitors as modulators of eCB signaling is limited by the significant role that COX-2 plays in prostaglandin (PG) synthesis.

Disclosed herein are SSCIs that inhibit COX-2 activity only when 2-AG or AEA, but not arachidonic acid, are used as substrates. Also disclosed herein are tests of the hypothesis that SSI-COX-2 increases brain eCB levels via a COX-2 dependent mechanism without affecting PG formation.

The SSCI Compound A increased brain AEA levels without affecting 2-AG levels 2 hours after i.p administration. Compound A did not affect brain or lung PG levels. Indomethacin also increased brain AEA levels, but profoundly decreased PG levels. The ability of Compound A to increase brain AEA levels was absent in COX-2 knockout (KO) mice, but was present in wild type littermates.

Compound A dose-dependently increased exploratory behavior in the open field, as well as center time exploration, which was most pronounced during minutes 40-60 of the one-hour test. These effects were similar to those seen with the fatty acid amide hydrolase (FAAH) inhibitor PF-3845 (N-3-pyridinyl-4-[[3-[[5-(trifluoromethyl)-2-pyridinyl]oxy]phenyl]methyl]-1-piperidinecarboxamide; CAS Number 1196109-52-0). Importantly, the observed behavioral effects of Compound A were absent in COX-2 KO mice and CB 1 receptor KO mice. Compound A also increased the number to light compartment entries in the light-dark box test. These data indicated that use of SSI-COX-2s is a viable strategy for in vivo augmentation of eCB signaling, and that the SSI-COX-2 Compound A has anxiolytic actions in animal models.

### Example 9

### Synthesis and Characterization of Exemplary Chiral Profens

### Raw Material Supplies and Instrumental:

Silica gel column chromatography was performed using Sorbent silica gel standard grade, porosity 60 Å, particle size 32-63 μm (230 ×450 mesh), surface area 500-600 m2/g, bulk density 0.4 g/mL, pH range 6.5-7.5, purchased from Sorbent Technologies (Atlanta, Ga., United States of America). Desmethylketoprofen Compound 3e was purchased from Toronto Research Chemicals. All other reagents, purchased from the Aldrich Chemical Company (Milwaukee, Wis., United States of America), were used without further purification. ¹H and ¹³C NMR were taken on a Bruker AV-I console operating at 400 MHz. Mass spectrometric analyses were performed on a Thermo Electron Surveyor pump TSQ 7000 instrument in ESI positive or negative ion mode. HPLC was performed with a Waters 2695 Separations Module with detection by a Waters 2487 Dual 2 Absorbance Detector at 260 nm and 285 nm. Light scattering was performed on a Sedex 75.

### General Synthetic Procedures:

A generalized scheme for generating SSCIs is presented in FIG. 17. It is understood, however, that any synthetic method can be employed for producing the presently disclosed SSCIs, and unless otherwise indicated, the method of synthesis is not to be considered limiting. Particular, non-limiting examples of employing the generalized scheme depicted in FIG. 17 are as follows:

General Procedure 1: Synthesis of Benzyl Aldehydes Compounds 2a-2d:

(Methoxymethyl)triphenylphosphonium chloride (4.0 eq) was suspended in dry THF (0.2 M) and cooled to 0° C. under an argon atmosphere. A solution of potassium tert-butoxide in THF (4.0 eq) was added slowly to the suspension and allowed to stir for 45 minutes at 0° C. The desired aryl aldehyde (e.g., Compound 1a; 3.0 mmol, 1.0 eq) was added dropwise and the solution was allowed to stir at room temperature for one hour. The reaction was quenched with saturated NH₄Cl and extracted 3× with EtOAc. The organic phase was dried with MgSO₄, filtered, and concentrated to yield an oil. The oil was then dissolved in a 5:2 THF:5 N HCl solution (0.2 M) and refluxed for one hour. The solution was cooled to room temperature, quenched with saturated NaHCO₃ and extracted 3× with EtOAc. The organic layer was dried with MgSO₄, filtered, and concentrated to yield an oil that was purified via silica gel column chromatography eluting with 30:1 hexanes:EtOAc.

General Procedure 2: Synthesis of Desmethylprofens Compounds 3a-3d:

The desired benzyl aldehyde (e.g., Compound 2a; 2.5 mmol, 1 eq) was dissolved in a 1:1 solution of H₂O:t-BuOH (0.1 M). In order, 2,3-dimethyl-2-butene (20 eq), KH₂PO₄ (2.0 eq) and sodium chlorite (2.5 eq) were added to the solution and allowed to stir for 40 minutes at room temperature. The reaction was extracted 3× with EtOAc and the combined organic layers were washed with a saturated NaCl solution, dried with MgSO₄ and concentrated to yield an off-white solid. The crude product was purified via silica gel column chromatography eluting with 9:1 hexanes:EtOAc to give a white solid.

General Procedure 3: Synthesis of Dimethyl Profen Methyl Esters:

A racemic profen (e.g., Compound 6a, 0.5 mmol, 1 eq) was dissolved in MeOH (0.2 M). A drop of concentrated H₂SO₄ was added and the solution was refluxed for two hours. The reaction was quenched with sat. NaHCO₃, extracted 3× with EtOAc, dried with MgSO₄ and concentrated to yield the methyl-ester protected racemic profen as an oil. This oil was dissolved in dry THF (0.2 M) and cooled to −78° C. under argon. A solution of LDA in THF (1.5 eq) was added slowly and allowed to stir at −78° C. for 30 minutes. The solution was warmed to 0° C. and HMPA (1.4 eq) was added slowly and allowed to stir at 0° C. for 30 minutes. Finally, 1.9 eq. of Mel was added dropwise to the solution and allowed to stir for 30 minutes at 0° C. and then thirty minutes at room temperature. The reaction was quenched with NH₄Cl, extracted 3× with EtOAc, dried with MgSO₄ and concentrated to yield an orange oil. The crude product was purified via silica gel column chromatography eluting with 9:1 hexanes:EtOAc to give an oil. Note: Compounds 3a-3e can be used as the starting material with this procedure as long as the equivalents of LDA, HMPA, and Mel are doubled from those described above.

General Procedure 4: Synthesis of Cyclopropyl Profen Methyl Esters:

A desmethylprofen (e.g., Compound 3a, 0.5 mmol, 1 eq) was dissolved in MeOH (0.2 M). A drop of concentrated H₂SO₄ was added and the solution was refluxed for two hours. The reaction was quenched with sat. NaHCO₃, extracted 3× with EtOAc, dried with MgSO₄ and concentrated to yield the methyl-ester protected profen as an oil. This oil was dissolved in dry THF (0.2 M) and cooled to −78° C. under argon. A solution of LDA in THF (2.5 eq) was added slowly and allowed to stir at −78° C. for 30 minutes. The solution was warmed to 0° C. and HMPA (2.0 eq) was added slowly and allowed to stir at 0° C. for 30 minutes. Finally, 1.5 eq. of 1,2-dibromoethane was added dropwise to the solution and allowed to stir for 30 minutes at 0° C. and then thirty minutes at room temperature. The reaction was quenched with NH₄Cl, extracted 3× with EtOAc, dried with MgSO₄ and concentrated to yield an orange oil. The crude product was purified via silica gel column chromatography eluting with 9:1 hexanes:EtOAc to give an oil.

General Procedure 5: Synthesis of Dimethyl (Compounds 4a-4e) and Cyclopropyl (Compounds 5a-5e) Profens:

A methyl-ester protected dimethyl profen or a methyl-ester protected cyclopropyl profen (0.2 mmol, 1.0 eq) was dissolved in dry THF (0.2 M). KOTMS (2.0 eq) was added to the reaction flask and refluxed for two hours. The slurry was quenched at room temperature with NH₄Cl, extracted 3× with EtOAc, dried with MgSO₄ and concentrated to yield an off-white solid. The crude product was purified via silica gel column chromatography eluting with 30:1 DCM:MeOH to give a dimethyl profen (e.g., Compound 4a) or a cyclopropyl profen (e.g., Compound 5a), respectively, as a white solid.

### Characterization of Compounds:

### 2-(2-fluoro-[1,1′-biphenyl]-4-yl)acetaldehyde (Compound 2a)

Compound 2a was prepared via general procedure 1 as a clear oil (56% yield). ¹H NMR (400 MHz, CDCl₃) δ 9.82 (t, 1H), 7.59 (m, 2H), 7.4-7.52 (m, 4H), 7.09 (m, 2H), 3.77 (d, 2H). MS m/z (ESI): calc. for C₁₄H₁₁FO [M-H]⁻ 213.08. found 213.4.

### 2-(6-methoxynaphthalen-2-yl)acetaldehyde (Compound 2b)

Compound 2b was prepared via general procedure 1 as a clear oil (48% yield). ¹H NMR (400 MHz, CDCl₃) δ 9.84 (t, 1H), 7.72-7.78 (m, 2H), 7.64 (s, 1H), 7.28-7.32 (m, 1H), 7.15-7.20 (m, 2H), 3.95 (s, 3H), 3.83 (d, 2H). MS m/z (ESI): calc. for C₁₃H₁₂O₂ [M-H]⁻ 199.08. found 199.4.

### 2-(4-isobutylphenyl)acetaldehyde (Compound 2c)

Compound 2c was prepared via general procedure 1 as a clear oil (77% yield). Note: intermediate and product are volatile when heated under reduced pressure. ¹H NMR (400 MHz, CDCl₃) δ 9.76 (t, 1H), 7.16 (d, 2H), 7.10 (d, 2H), 3.65 (d, 2H), 2.47 (d, 2H), 1.84 (m, 1H), 0.89 (d, 6H). MS m/z (ESI): calc. for C₁₂H₁₆O [M-H]⁻ 175.08. found 175.4.

### 2-(3-phenoxyphenyl)acetaldehyde (Compound 2d)

Compound 2d was prepared via general procedure 1 as a clear oil (75% yield). ¹H NMR (400 MHz, CDCl₃) δ 9.75 (t, 1H), 7.32 (m, 3H), 7.12 (m, 1H), 6.88-7.09 (m, 4H), 3.66 (d, 2H). MS m/z (ESI): calc. for C₁₄H₁₂O₂ [M-H]⁻ 211.07. found 211.27.

### 2-(2-fluoro-[1,1′-biphenyl]-4-yl)acetic acid (Compound 3a)

Compound 3a was prepared via general procedure 2 as a white solid (58% yield). ¹H NMR (400 MHz, CDCl₃) δ 7.55 (d, 2H), 7.35-7.5 (m, 4H), 7.1-7.2 (m, 2H), 3.7 (s, 3H). ¹³C NMR (400 MHz, CDCl₃) δ 176.1, 160.8, 159.2, 135.6, 134.7, 131.0, 129.1, 128.6, 128.2, 127.8, 125.5, 117.3, 117.1, 40.4. HRMS m/z (ESI): calc. for C₁₄H₁₁FO₂ [2M+Na]⁻ 481.1233. found 481.1245.

### 2-(6-methoxynaphthalen-2-yl)acetic acid (Compound 3b)

Compound 3b was prepared via general procedure 2 as a white solid (94% yield). ¹H NMR (400 MHz, CD₃OD) δ 7.75-7.65 (m, 3H), 7.38-7.32 (d, 1H), 7.3 (s, 1H), 7.11-7.08 (d, 1H), 3.9 (s, 3H), 3.72 (s, 2H). MS m/z (ESI): calc. for C₁₃H₁₂O₃ [M-H]⁻ 215.08. found 215.23.

### 2-(4-isobutylphenyl)acetic acid (Compound 3c)

Compound 3c was prepared via general procedure 2 as a white solid (89% yield). ¹H NMR (400 MHz, CDCl₃) δ 7.2 (d, 2H), 7.15 (d, 2H), 3.64 (s, 2H), 2.5 (d, 2H), 1.93-1.82 (m, 1H), 0.94-0.92 (d, 6H). MS rz/z (ESI): calc. for C₁₂H₁₆O₂ [M-H]⁻ 191.12. found 191.19.

### 2-(3-phenoxyphenyl)acetic acid (Compound 3d)

Compound 3d was prepared via general procedure 2 as a white solid (99% yield). ¹H NMR (400 MHz, CDCl₃) δ 7.35-7.23 (m, 3H), 7.12-7.07 (m, 1H), 7.0 (m, 3H), 6.98-6.88 (m, 2H), 3.62 (s, 3H). MS m/z (ESI): calc. for C₁₄H₁₂O₃ [M-H]⁻ 227.08. found 227.07.

### Methyl-2-(2-fluoro-[1,1′-biphenyl]-4-yl)-2-methylpropanoate

The methyl-ester protected form of Compound 4a was prepared via general procedure 3 as an oil (57% yield). ¹H NMR (400 MHz, CDCl₃) δ 7.54-7.50 (m, 2H), 7.47-7.3 (m, 4H), 7.17-7.1 (m, 2H), 3.7 (s, 3H), 1.61 (s, 6H). MS m/z (ESI): calc. for C₁₇H₁₇FO₂ [M+Na]⁺295.12. found 295.0.

### Methyl-2-(6-methoxynaphthalen-2-yl)-2-methylpropanoate

The methyl-ester protected form of Compound 4b was prepared via general procedure 3 as an oil (58% yield). ¹H NMR (400 MHz, CDCl₃) δ 7.54-7.50 (m, 2H), 7.47-7.3 (m, 41H), 7.17-7.1 (m, 2H), 3.7 (s, 3H), 1.61 (s, 6H). MS m/z (ESI): calc. for C₁₆H₁₈O₃ [M+Na]⁺281.13. found 281.07.

### Methyl-2-(4-isobutylphenyl)-2-methylpropanoate

The methyl-ester protected form of 4c was prepared via general procedure 3 as an oil (85% yield). ¹H NMR (400 MHz, CDCl₃) δ 7.25-7.2 (d, 2H), 7.1 (d, 2H), 3.68 (s, 3H), 2.47 (d, 2H), 1.9-1.8 (m, 1H), 1.6 (s, 6H), 0.93 (d, 6H). MS m/z (ESI): calc. for C₁₅H₂₂O² [M+Na]⁺257.16. found 257.07.

### Methyl-2-methyl-2-(3-phenoxyphenyl)propanoate

The methyl-ester protected form of Compound 4d was prepared via general procedure 3 as an oil (48% yield). ¹H NMR (400 MHz, CDCl₃) δ 7.35-7.25 (m, 3H), 7.13-6.97 (m, 5H), 6.87-6.82 (m, 1H), 3.70 (s, 3H), 1.58 (s, 6H). MS m/z (ESI): calc. for C₁₇H₁₈O₃ [M+Na]⁺ 293.13. found 293.0.

### Methyl-2-(3-benzoylphenyl)-2-methylpropanoate

The methyl-ester protected form of Compound 4e was prepared via general procedure 3 as an oil (64% yield). ¹H NMR (400 MHz, CDCl₃) δ 7.8 (m, 3H), 7.68-7.4 (m, 6H), 3.7 (s, 3H), 1.64 (s, 6H). MS m/z (ESI): calc. for C₁₈H₁₈O₃ [M+Na]⁺ 305.13. found 305.20.

Methyl-1-(2-fluoro-[1,1′-biphenyl]-4-yl)cyclopropanecarboxylate: The methyl-ester protected form of Compound 5a was prepared via general procedure 4 as an oil (28% yield). ¹H NMR (400 MHz, CDCl₃) δ 7.57-7.49 (m, 2H), 7.45-7.33 (m, 4H), 7.2-7.12 (m, 2H), 3.66 (s, 3H), 1.66-1.62 (dd, 2H), 1.25-1.20 (dd, 2H). MS m/z (ESI): calc. for C₁₇H₁₅FO₂ [M+Na]⁺ 293.11. found 293.10.

### Methyl-1-(6-methoxynaphthalen-2-yl)cyclopropanecarboxylate

The methyl-ester protected form of Compound 5b was prepared via general procedure 4 as an oil (22% yield). ¹H NMR (400 MHz, CDCl₃) δ 7.70-7.68 (m, 3H), 7.46-7.44 (m, 1H), 7.15-7.12 (m, 2H), 3.92 (s, 3H), 3.62 (s, 3H), 1.67-1.64 (dd, 2H), 1.28-1.24 (dd, 2H). MS m/z (ESI): calc. for C₁₆H₁₆O₃ [M⁺ Na]279.11. found 279.09.

### Methyl-1-(4-isobutylphenyl)cyclopropanecarboxylate

The methyl-ester protected form of Compound 5c was prepared via general procedure 4 as an oil. Note: the product could not be isolated as a pure compound after column chromatography, but the impurities did not affect the next reaction (41% crude yield). ¹H NMR (400 MHz, CDCl₃) δ 7.27-7.2 (d, 2H), 7.18-7.13 (d, 2H), 3.68 (s, 3H), 2.44-2.4 (d, 2H), 1.88-1.78 (m, 1H), 1.6-1.55 (dd, 211), 1.28-1.24 (dd, 2H). MS m/z (ESI): calc. for C₁₅H₂₀O₂ [M+Na]⁺ 255.15. found 255.12.

### Methyl-1-(3-phenoxyphenyl)cyclopropanecarboxylate

The methyl-ester protected form of Compound 5d was prepared via general procedure 4 as an oil (17% yield). ¹H NMR (400 MHz, CDCl₃) δ 7.36-7.21 (m, 3H), 7.12-7.0 (m, 5H), 6.88-6.84 (m, 1H), 3.63 (s, 3H), 1.59-1.57 (dd, 2H), 1.19-1.16 (dd, 2H). MS m/z (ESI): calc. for C₁₇H₁₆O₃ [M+Na]⁺291.11. found 291.09.

### Methyl-1-(3-benzoylphenyl)cyclopropanecarboxylate

The methyl-ester protected form of Compound 5e was prepared via general procedure 4 as an oil (18% yield). ¹H NMR (400 MHz, CDCl₃) δ 7.82-7.78 (m, 3H), 7.7-7.66 (m, 1H), 7.61-7.54 (m, 2H), 7.52-7.39 (m, 3H), 3.64 (s, 3H), 1.67-1.63 (d, 2H), 1.25-1.21 (dd, 2H). MS m/z (ESI): calc. for C₁₇H₁₆O₃ [M+Na]⁺303.11. found 303.15.

### 2-(2-fluoro-[1,1′-biphenyl]-4-yl)-2-methylpropanoic acid (Compound 4a)

Compound 4a was prepared via general procedure 5 as a white solid (84% yield). ¹H NMR (400 MHz, CDCl₃) δ 7.56-7.5 (m, 2H), 7.45-7.36 (m, 4H), 7.17-7.08 (m, 2H), 1.65 (s, 6H). ¹³C NMR (400 MHz, CDCl₃) δ 182, 161, 158, 146, 135, 131, 129, 128.5, 128, 127.8, 122, 114.3, 114, 46, 27 (two carbons). HRMS m/z (ESI): calc. for C₁₆H₁₅FO₂ [2M+Na]-537.1859. found 537.1871.

### 2-(6-methoxynaphthalen-2-yl)-2-methylpropanoic acid (Compound 4b)

Compound 4b was prepared via general procedure 5 as a white solid (92% yield). ¹H NMR (400 MHz, CDCl₃) δ 7.74-7.71 (m, 3H), 7.51-7.49 (m, 1H), 7.17-7.12 (2H), 3.93 (s, 3H), 1.70 (s, 6H). MS m/z (ESI): calc. for C₁₅H₁₆O₃ [M-H]⁻ 243.110 found 243.31.

### 2-(4-isobutylphenyl)-2-methylpropanoic acid (Compound 4c)

Compound 4c was prepared via general procedure 5 as a white solid (92% yield). ¹H NMR (400 MHz, CDCl₃) δ 7.33-7.3 (d, 2H), 7.11-7.07 (d, 2H), 2.58-2.53 (d, 2H), 1.91-1.80 (m, 1H), 1.60 (s, 2H), 0.92-0.89 (d, 6H). MS m/z (ESI): calc. for C₁₄H₂₀O₂ [M-H]⁻ 219.15. found 219.13.

### 2-methyl-2-(3-phenoxyphenyl)propanoic acid (Compound 4d)

Compound 4d was prepared via general procedure 5 as a white solid (95% yield). ¹H NMR (400 MHz, CDCl₃) δ 7.36-7.24 (m, 3H), 7.13-6.98 (m, 5H), 6.85-6.80 (m, 1H), 1.58 (s, 6H). MS m/z (ESI): calc. for C₁₆H₁₆O₃ [M-H]⁻ 255.11. found 255.07.

### 2-(3-benzoylphenyl)-2-methylpropanoic acid (Compound 4e)

Compound 4e was prepared via general procedure 5 as a white solid (89% yield). ¹H NMR (400 MHz, CDCl₃) δ 7.88-7.76 (m, 3H), 7.68-7.53 (m, 3H), 7.47-7.39 (m, 3H), 1.64 (s, 6H). MS m/z (ESI): calc. for C₁₇H₁₆O₃ [M-H]⁻ 267.11. found 267.13.

### 1-(2-fluoro-[1,1′-biphenyl]-4-yl)cyclopropanecarboxylic acid (Compound 5a)

Compound 5a was prepared via general procedure 5 as a white solid (77% yield). ¹H NMR (400 MHz, CDCl₃) δ 7.54-7.52 (m, 2H), 7.45-7.36 (m, 4H), 7.23-7.15 (m, 2H), 1.74-1.71 (dd, 2H), 1.34-1.31 (dd, 2H). ¹³C NMR (400 MHz, CDCl₃) δ 181, 161, 158, 140, 135.5, 131, 129, 128.5, 128.3, 127.7, 126.4, 118.4, 118.2, 28, 18 (two carbons). HRMS m/z (ESI): calc. for C₁₆H₁₃FO₂ [M-H]⁻ 255.0827, found 255.0835.

### 1-(6-methoxynaphthalen-2-yl)cyclopropanecarboxylic acid (Compound 5b)

Compound 5b was prepared via general procedure 5 as a white solid (78% yield). ¹H NMR (400 MHz, CDCl₃) δ 7.7-7.68 (m, 3H), 7.48 (m, 1H), 7.14-7.11 (m, 2H), 3.91 (s, 3H), 1.74-1.71 (dd, 2H), 1.36-1.34 (dd, 2H). MS m/z (ESI): calc. for C₁₅H₁₄O₃ [M-H]⁻ 241.09. found 241.2.

### 1-(4-isobutylphenyl)cyclopropanecarboxylic acid (Compound 5c)

Compound 5c was prepared via general procedure 5 as a white solid. ¹H NMR (400 MHz, CDCl₃) δ 7.27-7.24 (d, 2H), 7.07-7.03 (d, 2H), 2.47-2.41 (d, 2H), 1.89-1.8 (m, 1H), 1.66-1.62 (dd, 2H), 1.25-1.21 (dd, 2H), 0.91-0.86 (d, 6H). MS m/z (ESI): calc. for C₁₄H₁₈O₂ [M-H]⁻ 217.13. found 217.30.

### 1-(3-phenoxyphenol)cyclopropanecarboxylic acid (Compound 5d)

Compound 5d was prepared via general procedure 5 as a white solid (59% yield). ¹H NMR (400 MHz, CDCl₃) δ 7.35-7.19 (m, 3H), 7.09-6.96 (m, 5H), 6.87-6.83 (m, 1H), 1.7-1.67 (dd, 2H), 1.3-1.28 (dd, 2H). MS m/z (ESI): calc. for C₁₆H₁₄O₃ [M-H]⁻ 253.09. found 253.27.

### 1-(3-benzoylphenyl)cyclopropanecarboxylic acid (Compound 5e)

Compound 5e was prepared via general procedure 5 as a white solid (59% yield). ¹H NMR (400 MHz, CDCl₃) δ 7.83-7.81 (m, 3H), 7.73-7.68 (m, 1H), 7.61-7.56 (m, 2H), 7.49-7.39 (m, 3H), 1.74-1.71 (dd, 2H), 1.33-1.30 (dd, 2H). MS m/z (ESI): calc. for C₁₇H₄O₃ [M-H]⁻265.09. found 265.29.

Separation of Compounds 7d and 7e:

A racemic profen (e.g., Compound 6d) was dissolved in a 90:10 solution of hexanes:IPA with 0.1% TFA for a final concentration of 10 mg/mL. An aliquot of 30 L was injected onto a CHIRALCEL® AD column using an isocratic gradient of 90:10 hexane:IPA with 0.1% TFA. Compound 7d eluted at 7.7 minutes and Compound 7e eluted at 14.2 minutes. Fractions were collected manually. The identities of the enantiomers were identified after separation via optical rotation (see e.g., Brebion et al., 2003; Monti et al., 2005). Enantiomeric excess was determined by re-injecting the separated enantiomers (e.g., Compound 7d) and integrating each peak (FIG. 18). Profens Compounds 7d and 7e were isolated at 98% ee and 99% ee, respectively.

### Example 10

### Development and Evaluation of Achiral Profen Probes

Disclosed herein are investigations into developing an in vivo probe to determine the effects of blocking 2-AG and AEA, but not AA, oxygenation by COX-2. The (R)-profens are active in vitro and in intact cells, but undergo uni-directional inversion to the (S)-enantiomers (which inhibit AA oxygenation) in vivo.

This enantiomerization is an enzymatic process proceeding through an acyl coenzyme A thioester intermediate (Woodman et al., 2011). One approach to eliminate this complication is to synthesize profen derivatives that are unable to invert in vivo. Described herein is the synthesis and in vitro evaluation of achiral NSAIDs based on five profen scaffolds that exhibit substrate-selective behavior (i.e., flurbiprofen, naproxen, ketoprofen, fenoprofen, and ibuprofen), with the most potent and substrate-selective inhibitors being further examined in activated RAW264.7 cells and evaluated for stability in animal models (Davie et al., 1092; Ossipov et al., 2000; Duggan et al., 2011).

Three achiral derivatives of each profen scaffold were synthesized by altering the substitution pattern at the methyl-bearing stereocenter to contain a desmethyl (two hydrogens), dimethyl (two methyls), or cyclopropyl (two methylenes) group. The synthetic route is outlined in FIG. 17 and described in detail in the Supporting Information.

Compounds 3a-e, 4a-e, and 5a-e were tested for their ability to inhibit the COX-2-dependent oxygenation of 2-AG and AA in vitro using the method described in Duggan et al., 2011. The IC₅₀ value for 2-AG oxygenation was determined for each compound. Since none of the compounds potently inhibited AA oxygenation, percent inhibition was determined at the highest concentration employed. The data are presented in Table 5.

TABLE 5
Inhibition of mCOX-2 Dependent Oxygenation of 2-AG and
AA by Achiral Profens In Vitro^{a}

| Compound | 2-AG IC₅₀ (μM)^{b} | AA % Inhibition^{c} |
|---|---|---|
| 3a | 0.11 +/− 0.02 | 7 |
| 3b | 1.4 +/− 0.4 | 16 |
| 3c | 5.5 +/− 0.8 | 14 |
| 3d | 6.1 +/− 0.5 (68%) | 24 |
| 3e | 0.5 +/− 0.1 | 10 |
| 4a | 1.1 +/− 0.4 | 5 |
| 4b | — (12%) | 7 |
| 4c | — (5%) | 5 |
| 4d | 7.3 +/− 0.5 (65%) | 17 |
| 4e | 3.0 +/− 1.2 | 32 |
| 5a | 2.3 +/− 1.2 (87%) | 0 |
| 5b | 3.4 +/− 1.2 (77%) | 6 |
| 5c | — (45%) | 10 |
| 5d | 5.0 +/− 0.8 | 15 |
| 5e | 4.1 +/− 1.4 (75%) | 10 |

^{a}IC₅₀ values were determined by incubating five concentrations of inhibitor and a solvent control in DMSO with purified murine COX-2 (40 nM) for three min followed by addition of 2-AG or AA (5 μM) at 37° C. for 30 s.
^{b}Mean ± standard deviation (n = 6); dash (—) indicates <50% inhibition of 2-AG oxygenation at 10 μM inhibitor. Numbers in parentheses indicate maximum inhibition (when not equal to 100%) at 10 μM inhibitor.
^{c}% inhibition of AA oxygenation measured at 10 μM inhibitor.

Several interesting observations were made. First, it appeared that inhibition of 2-AG oxygenation was dependent on the size of the substituents on the α-carbon, with smaller groups exhibiting higher potency. In general, desmethylprofen Compounds 3a-e had lower 2-AG IC₅₀ values than the dimethyl and cyclopropyl profens, Compounds 4a-e and 5a-e, respectively. Profens, 4a-e and 5a-e, possessed approximately equivalent IC₅₀ values against 2-AG, reflecting their similar steric bulk.

Second, regardless of the substitution on the α-carbon, flurbiprofen derivatives exhibited the lowest 2-AG IC₅₀ values compared to the other profen scaffolds in the same class. Flurbiprofen derivative Compound 3a has a 2-AG IC₅₀ value of 0.11 μM, significantly lower than the next best achiral inhibitor, Compound 3e (0.5 μM). The ketoprofen scaffold was the next most potent, followed by the naproxen and then fenoprofen scaffolds. The achiral ibuprofen derivatives were the weakest inhibitors of 2-AG oxygenation, with the best ibuprofen-based inhibitor, Compound 3c, possessing a relatively weak 2-AG IC₅₀ value of 5.5 μM.

Third, the flurbiprofen scaffold offers the best substrate selectivity of the scaffolds evaluated. Each flurbiprofen derivative had lower or equal inhibition of AA oxygenation than the other derivatives in each class while also having a lower IC₅₀ for 2-AG.

Finally, while every achiral compound besides Compound 5a showed some inhibition of AA oxygenation, the desmethyl class of compounds was the only class where each inhibitor displayed greater than 5% AA inhibition. Within this class it appears that the gains in potency of 2-AG inhibition come at a cost of substrate selectivity.

The enantiomers of racemic fenoprofen and ketoprofen were also resolved to determine their ability to act as substrate-selective inhibitors in an in vitro assay. (R)-Fenoprofen (Compound 7d) and (R)-ketoprofen (Compound 7e) possessed weak inhibitory effects on both 2-AG and AA oxygenation (Table 6). (S)-Fenoprofen and (S)-ketoprofen exhibited 2-AG IC₅₀ and percent AA inhibition values similar to those of racemic fenoprofen (Compound 6d) and ketoprofen (Compound 6e), respectively. These data suggest that the activity of the racemates originate from their (S)-enantiomers and demonstrate that a (R)-profen scaffold does not guarantee substrate selectivity.

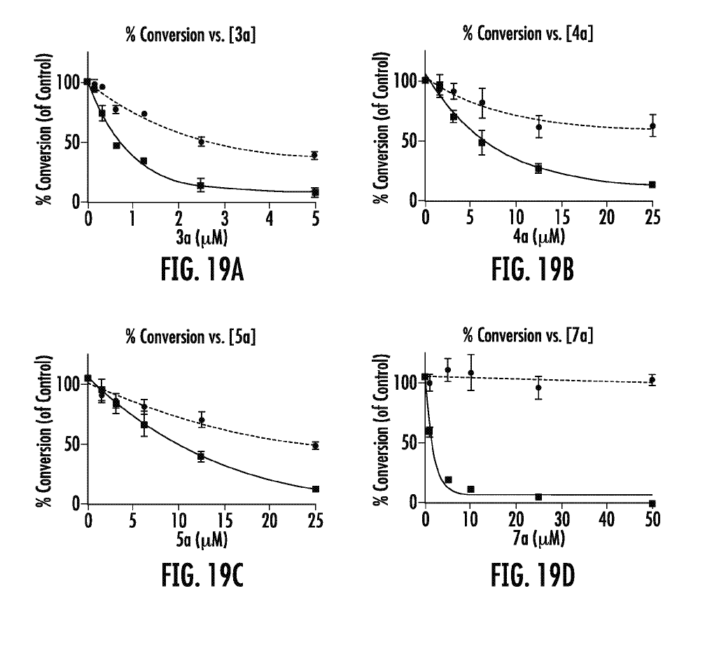

Flurbiprofen derivatives, Compounds 3a, 4a, 5a, and 7a were evaluated for their ability to selectively inhibit COX-2 dependent oxygenation of 2-AG over AA in intact cells (FIG. 19). RAW 264.7 macrophages were stimulated with lipopolysaccharide and interferon γ to generate endogenous sources of AA and 2-AG. Two hours after stimulation, varying doses of inhibitor were added to the cell media and 6 hr after stimulation the media were collected and analyzed by LC-MS-MS for prostaglandin levels. The 2-AG IC₅₀ values in RAW cells for each achiral compound were 5-fold higher than the values found in the in vitro studies, while Compound 7a displayed a 20-fold increase. Although <10% inhibition of AA oxygenation was observed for each achiral flurbiprofen derivative in vitro, significant inhibition was observed in RAW cells (60% AA inhibition for Compound 3a, 40% for Compound 4a, 55% for Compound 5a). Of particular interest was the observation of AA inhibition by Compound 5a, the only achiral derivative not to exhibit AA inhibition with purified COX-2.

The pharmacokinetic properties of the two most potent and selective profens screened to date, Compounds 3a and 7a, were also evaluated in C57BL/6 mice. Animals (n=4) received intraperitoneal (i.p.) injections of 10 mg/kg Compound 3a or Compound 7a in 4% DMSO/2% Tween 80 in saline at 0, 8, 24, 32, 48 and 56 hr, with blood collected at 72 hr. Plasma was analyzed for the dosed compound and, in the case of Compound 7a, its stereoinversion product (S-flurbiprofen). When compared to Compound 7a, Compound 3a appeared to exhibit less metabolic stability. Sixteen hr after the last injection, Compound 3a was not detected in mouse plasma, whereas Compound 7a gave an average plasma concentration of 20 μM. The concentration of the stereoinversion product was 31 μM.

Mice underwent acute dosing of Compound 3a at shorter time points to better estimate the compound's half-life in mouse plasma. Blood samples were collected at 1, 2 and 4 hr after a single injection. Analysis of the resultant plasma levels revealed that the 1 hr time point had an average Compound 3a plasma concentration of 68 μM (n=3) followed by concentrations of 50 (n=2) and 6 μM (n=3) at the 2 and 4 hr time points, respectively. These data suggested that Compound 3a possessed a half-life similar to ibuprofen (t_{1/2≈}2-3 hr).

X-ray crystallography was used to study the interaction between achiral profens and COX-2. A complex of mCOX-2 and Compound 3a, using a previously described crystallization procedure, diffracted to 2.81 Å. The carboxylic acid of the ligand forms salt bridges with Arg-120 and Tyr-355. The biphenyl moiety of Compound 3a is deeply buried into the hydrophobic channel and interacts with several residues, such as Val-349, Phe-381, Trp-387, Phe-518, Met-522, Val-523, and Leu-531.

When the crystal structures of (R)-flurbiprofen and Compound 3a bound to mCOX-2 are overlaid, the two profens have the same general binding orientation in the mCOX-2 active site (0.24 Å RMSD). This orientation allows for the formation of a salt bridge with Arg-120, an interaction crucial for binding. Establishing the salt bridge with Arg-120 requires the carboxylic acid and α-carbon stereocenter of flurbiprofen to bind near the constriction site of the COX-2 binding pocket, a sterically congested region. Relief of this congestion is likely responsible for the increased inhibition of 2-AG oxygenation upon introduction of smaller substituents at the flurbiprofen α-carbon. Yet, there is a trade-off between potency and selectivity. Comparing the RAW cell data of Compounds 3a and 7a suggests that reducing the steric bulk at the α-carbon (i.e., converting the α-methyl group of Compound 7a into the hydrogen present in Compound 3a) increases potency for 2-AG and AA inhibition, resulting in less substrate selectivity. While not wishing to be bound by any particular theory of operation, there appear to be favorable interactions promoting substrate selectivity that the more sterically demanding Compound 7a can utilize, but are unavailable to the smaller Compound 3a.

The data presented here led to several conclusions about achiral profen COX-2 inhibitors. First, achiral profens demonstrated substrate-selective behavior in vitro and in a cellular setting. Second, smaller α-carbon substituents (i.e., hydrogens) resulted in more potent, but less selective inhibitors than more sterically demanding groups (i.e., dimethyl, cyclopropyl). Finally, inhibitor potency and selectivity were dependent on the profen's aryl scaffold, with some scaffolds (e.g. flurbiprofen) offering superior behavior relative to others (e.g., ibuprofen). In addition, the most potent compound, desmethylflurbiprofen (Compound 3a), possessed metabolic stability on par with ibuprofen. These results indicated that achiral profens can act as in vivo probes of substrate-selective COX-2 inhibition.

General Procedure for In Vitro Assay:

Desired concentrations of inhibitor in DMSO or blank (DMSO) were incubated with mCOX-2 (40 nM) for 3 minutes in 100 mM Tris-HCl with 0.5 mM phenol, pH 8.0 at 37° C. After pre-incubation of enzyme and inhibitor, 5 μM of AA or 2-AG was added at 37° C. Thirty seconds later, the reaction was quenched with 200 μL of ice-cold ethyl acetate containing 0.5% acetic acid (v/v) and 1 μM PGE₂-d₄ and PGE₂-G-d₅. The solution was then vortexed and the aqueous layer frozen. The organic layer was removed and evaporated to near-dryness under nitrogen. The samples were reconstituted in 1:1 MeOH:H₂O and chromatographed using a LUNA® C18(2) column (50 2 mm, 3 μm; Phenomenex, Torrance, Calif., United States of America). The elution method was run at a flow rate of 0.4 mL/min and consisted of a linear gradient 20-95% Solvent B over five minutes, followed by a one minute linear gradient 95-20% Solvent B and a thirty second isocratic gradient at 20% Solvent B. Solvent A contained 5 mM ammonium acetate pH=3.5 and Solvent B was 6% Solvent A in ACN. MS/MS was conducted on a Quantum triple quadrupole mass spectrometer operated in positive ion mode utilizing a selected reaction monitoring method with the following transitions: m/z 370-+317 for PGE₂/D₂, m/z 374-321 for PGE₂-d₄, m/z 444-+391 for PGE₂/D₂-G and m/z 449-396 for PGE2/D₂-G-d₅. Analyte peak areas were normalized to the appropriate internal standard to determine the amount of product formation, and inhibition was determined by normalization to a DMSO control. Results are presented in Table 6.

TABLE 6
Inhibition of mCOX-2 Dependent Oxygenation of 2-AG
and AA by Chiral Profens In Vitro^{a}

| | IC_{50 (μM)}^{b} | % Inhibition^{c} |
|---|---|---|
| Compound | 2-AG | AA |
| 7a^{d} | 0.08 | 0 |
| 7b^{d} | 3 | 0 |
| 7c^{d} | 10 | 0 |
| 7d | — (25%) | 10 |
| 7e | 7.3 +/− 0.4 (56%) | 20 |
| (S)-fenoprofen | 0.9 +/− 0.5 | 30 |
| (S)-ketoprofen | 0.2 +/− 0.04 | 55 |
| 6d^{e} | 0.9 +/− 0.2 | 20 |
| 6e^{e} | 0.3 +/− 0.04 | 50 |

^{a}IC₅₀ values were determined by incubating five concentrations of inhibitor and a solvent control in DMSO with purified murine COX-2 (40 nM) for three min followed by addition of 2-AG or AA (5 μM) at 37° C. for 30 seconds.
^{b}Mean ± standard deviation (n = 6); dash (—) indicates <50% inhibition of 2-AG oxygenation at 10 μM inhibitor. Numbers in parentheses indicate maximum inhibition (when not equal to 100%) at 10 μM inhibitor.
^{c}% inhibition of AA oxygenation measured at 10 μM inhibitor.
^{d}Data taken from reference 10.
^{e}n = 3.

General Procedure for RAW Cells Assay:

RAW 264.7 macrophages were plated onto 60×15 mm collagen coated dishes at 2 million cells per dish in DMEM with GLUTAMAX® tissue culture medium supplement with 10% HI-FBS. The cells were then stimulated overnight with 20 ng/ml of granulocyte-macrophage colony-stimulating factor. The media was replaced in the morning with DMEM with GLUTAMAX® and the cells were stimulated with 100 ng/ml lipopolysaccharide and 20 units/ml interferon γ. Two hours after stimulation varying doses of inhibitor were added to the cell media and 6 hours after stimulation the media was collected and extracted in acidified ethyl acetate spiked with deuterated internal standards. The organic layer was dried down under a stream of nitrogen gas and reconstituted in 1:1 water:methanol and analyzed by LC-MS/MS for prostaglandin levels using the same instrumentation, column, solvents and conditions as described for the in vitro assay above.

General Procedure for In Vivo Mouse Assay:

The following animal protocol was approved by the Vanderbilt University Institutional Animal Care and Use Committee, and all procedures were performed in accordance with national guidelines and regulations. Male C57BL/6 mice were dosed by intraperitoneal (i.p.) injection with 10 mg/kg of compound. For Compound 3a at the designated time points of one hour, two hours, and four hours, mice were euthanized. Separately, mice were dosed with either Compound 3a or Compound 7a i.p. with 10 mg/kg at 0, 8, 24, 32, 48 and 56 hours. Blood was collected from the mice at 72 hours. All blood samples were immediately collected into heparinized syringes by cardiac puncture. The 16 hour time points for Compounds 7a, 3a, and (S)-flurbiprofen were extracted from murine plasma as follows: 200 μL plasma was diluted 5:1 (v.v) with 1% acetic acid (aqueous) then 100 μL acetonitrile and 1.2 mL hexane were added. The plasma samples were mixed well and centrifuged to promote phase separation. The upper, organic layer was removed and dried. The samples were reconstituted in 9:1 isopropyl alcohol:hexane (v:v) immediately prior to analysis on an isocratic system where the mobile phase was 98:2 isopropyl alcohol:hexane with 0.1% acetic acid. Chiral separation occurred on a CHIRALCEL® OD column (25×0.46 cm) and fluorescence detection was employed (λₑₓ=248 nm, λₑₘ=312 nm). The one, two and four hr timepoints for Compound 3a were extracted from murine plasma as follows: 200 L murine plasma was spiked with flurbiprofen (internal standard) and diluted with 1 mL 1% acetic acid (aqueous) and loaded onto a pre-conditioned OASIS HLB solid phase extraction column. The OASIS HLB cartridge had been conditioned with 3 mL methanol and 2 mL of 1% acetic acid (aq). The loaded cartridge was washed with 3 mL of 1% acetic acid (aq) and 1.5 mL of 1% acetic acid (aq) with 20% methanol. The cartridge was eluted with 2 mL methanol and the eluant was dried. The samples were reconstituted in 100 μL methanol and 50 μL water immediately prior to analysis on a gradient of 30% B to 75% B over 11 min, where A=water and B=1:1 methanol:acetonitrile (v:v), each with 0.1% acetic acid. Separation occurred on a Zorbax C18 column (15×0.21 cm) and fluorescence detection was employed (λₑₓ=285 nm, λₑₘ=340 nm).

General Procedure for Crystallization, X-Ray Data Collection, Structure Determination and Refinement:

Crystallization was performed as described in Duggan et al., 2011 with modest modification. Purified mCOX-2 (10 mg/mL) was reconstituted with a 2-fold molar excess of Fe³+-protoprophyrin IX. After dialysis against 20 mM sodium phosphate buffer pH 6.7, 100 mM NaCl, 0.01% NaN₃, 0.6% (w/v) β-OG, β-OG concentration was adjusted to 1.2% and 10-fold molar excess of Compound 3a was added prior to setup. Crystallization was performed using hanging drop vapor diffusion method by combining 3.5 μL of the protein solution with 3.5 μL 50 mM EPPS pH 8.0, 80-120 mM MgCl₂, 20-25% (v/v) PEG MME-550 equilibrating over reservoir solution containing 0.5 mL of 50 mM EPPS pH 8.0, 100-120 mM MgCl₂, 20-25% (v/v) PEG MME-550 at 291 K. Crystals were mounted after about 3 weeks growth and transferred to the stabilization solution[50 mM EEPS, 28% (v/v) PEG MME 550, 100 mM MgCl₂] for about 10 seconds and flash frozen for crystal transportation.

Data sets were collected on an ADSC Quantum-315 CCD using the synchrotron radiation X-ray source at a wavelength of 0.97929 Å with 100 K liquid nitrogen streaming at beamline 24-ID-E of the Advance Photon Source at the Argonne National Laboratory. Diffraction data were collected and processed with HKL-2000 (Otwinowski et al., 1997). Initial phases were determined by molecular replacement using a search model (PDB 3NT1) with Phaser software. Solution with four molecules in the asymmetric unit was obtained. The models were improved with several rounds of model building in COOT software (Emsley et al., 2010) and CCP4 suite (Patterton et al., 2003). There were no significant structural differences evident among the monomers. Global non-crystallographic symmetry was applied during the refinement. The ligand was built using SMILE and waters were adding from COOT (Emsley et al., 2010). The final model has R_{work} and R_{free} 0.240 and 0.289, respectively. The potential of phase bias was excluded by simulated annealing using Phenix software (Adams et al., 2010). The values of the Ramachandran plot for the final refinement of the structure were obtained with PROCHECK software (Laskowski et al. 1993). Data collection and refinement statistics are reported in Table 7. The atomic coordinates and structure factor have been deposited in the Protein Data Bank under the access id 4FM5 (see the website of the RCSB PDB on the World Wide Web; www[dot]rcsb[dot]org). The illustrations were prepared using the coordinates of monomer A with Pymol (Schrodinger, LLC).

TABLE 7
Data Collection and Refinement Statistics of mCOX-2:
Compound 3a Complex

| | mCOX-2:3a complex (PDB: 4FM5) |
|---|---|
| Data collection | |
| Space group | P2₁2₁2 |
| Cell dimensions | |
| a, b, c (Å) | 181.63, 135.70, 125.15 |
| α, β, γ (°) | 90, 90, 90 |
| Resolution (Å) | 50.0-2.80 (2.90-2.80)* |
| Total reflections | 303710 |
| Unique reflections | 74531 |
| R_{sym} | 0.094 (0.498) |
| Mean I/σ(I) | 6.92 (2.03) |
| Completeness (%) | 98.46 (81.56) |
| Multiplicity | 4.1 (4.0) |
| Wilson B-factor | 48.97 |
| Refinement | |
| Resolution (Å) | 49.86-2.81 (2.89-2.81)* |
| R_{work}/R_{free} | 0.240/0.289 (0.335/0.367) |
| No. atoms | 18701 |
| Protein | 17896 |
| Ligand | 624 |
| Solvent | 181 |
| B-factors | 45.9 |
| Protein | 45.4 |
| Ligand | 62.7 |
| Solvent | 37.3 |
| RMS (bonds, Å) | 0.016 |
| RMS (angles, °) | 1.87 |
| Ramachandran Plot | |
| Preferred | 95.91% (2110) |
| Allowed | 3.95% (87) |
| Outliers | 0.14% (3) |

The values in parentheses are for the highest resolution shell;

$${R}_{sym} = \frac{{\sum }_{hkl}^{ } {\sum }_{i}^{ } \vert {I}_{i} ( hkl ) - \overline{{I}_{i} ( hkl )} \vert}{{\sum }_{hkl}^{ } {\sum }_{i}^{ } {I}_{i} ( hkl )} \times 100 \% ; R = \frac{{\sum }_{hkl}^{ } ‖ {F}_{o} \vert - \vert {F}_{c} ‖}{{\sum }_{hkl}^{ } \vert {F}_{o} \vert} \times 100 \% ,$$

where Fₒ and Fₑ are the observed and calculated structure factors, R_{free}=test set 5.0%.

This crystal structure has been deposited in the Protein Data Bank (PDB: 4FM5).

### Materials and Methods for EXAMPLEA 11-16

Materials.

Indomethacin was purchased from Sigma Aldrich Chemical (St. Louis, Mo., United States of America). NS-398, SC-560, JZL-184, PF-3845, URB597, PGE₂-d₄, AA-d₅, 2-AG-d₅, and AEA-d₅ were purchased from Cayman Chemical (Ann Arbor, Mich., United States of America). Compound A (2-[1-(4-Chloro-benzoyl)-5-methoxy-2-methyl-1H-indol-3-yl]-1-morpholin-4-yl-ethanone) was synthesized as described in Kalgutkar et al., 2000b.

In Vitro Enzyme Purification and Activity Assays.

Wildtype, R120Q, and Y355F COX-2 were expressed in insect cells and purified as described in Rowlinson et al., 1999. In vitro COX-2 inhibition assays were performed as described in Duggan et al., 2011. The RAW 264.7 macrophage inhibition assay was performed as described in Rouzer & Marnett, 2006. MAGL was purified using BL21(DE3) pLysS E. coli transformed with pET-45b(+) plasmid containing human MGL-His.

Cells were grown at 37° C. to a density of 0.7 OD and then protein expression induced with IPTG (1 mM). Cells were harvested 4 hr later and proteins purified using Ni-NTA Agarose (Qiagen) as described in Blankman et al., 2007. After purification, the protein was dialyzed overnight at 4° C. into buffer containing 0 mM HEPES and 0.01% Triton X-100. MAGL inhibition was assessed as described in Blankman et al., 2007. Humanized rat FAAH was a generous gift of R. Stevens and B. Cravatt (The Scripps Research Institute). FAAH inhibition was assessed as described in Ahn et al., 2009. Human DAGLα in pcDNA3.1D was expressed in HEK293T cells for 24 hours then harvested and membranes prepared as described in Pedicord et al., 2011. DAGLα activity was assessed using 5 μg of membrane protein in a 50 μL reaction of assay buffer containing 50 mM MES (pH 6.5) and 2.5 mM CaCl₂. 1-steroyl-2-arachidonoyl glycerol (SAG) was added directly from a 100% methanol stock for a final concentration of 250 μM (5% final concentration of methanol in reaction). The reaction was terminated after 15 minutes by the addition of 200 μl methanol containing 125 pmol 2-AG-d₈. The samples were spun down at 2000×g and the soluble material injected directly for LC/MS/MS analysis.

Animals.

5-7 week old male ICR mice were used for all experiments with the exception of knockout animals (Harlan, Indianapolis, Ind., United States of America). Mice were housed 5 per age. All behavioral tests were conducted during the light cycle between 0900 and 1700. KO and WT littermate controls for FAAH^{(-/-)} and COX-2^{(-/-)} mice were derived from heterozygote breeding pairs, bred and genotyped as described in Uddin et al., 2011. CB1^{(-/-)} mice were bred from homozygote breeding pairs and genotyped as described in Pan et al., 2008. Mice were group-housed on a 12:12 light-dark cycle (lights on at 06:00), with food and water available ad libitum. All animal studies were approved by the Vanderbilt Institutional Animal Care and Use Committee and conducted in accordance with the Guide for the Care and Use of Laboratory Animals of the United States National Institutes of Health.

Tissue Preparation and Lipid Extraction.

Mice were sacrificed by cervical dislocation and decapitation. The brain, lungs, liver, stomach, small intestines, and kidneys were then rapidly removed and frozen on a metal block in dry ice. The tissue was then placed in a tube and stored at −80° C. until extraction, usually one day after harvesting. For PG and eCB analysis, lipid extraction from tissue was carried out as described in Patel et al., 2009.

Open Field Test.

Animals were tested for open-field activity in a novel environment one hour after i.p. injection of compound as described in Sumislawski et al., 2011. Briefly, one-hour sessions were performed using automated experimental chambers (27.9 27.9 cm; MEDOFA-510; MED Associates, Ga., Vermont, United States of America) under constant illumination within a sound-attenuated room. Analysis of open field activity was performed using Activity Monitor v5.10 (MED Associates).

Light-Dark Box.

Anxiety responses were assessed in a plastic light-dark chamber measuring 20×20 cm. Half of the chamber was opaque with a black Plexiglas insert; the other half remained transparent. Photocells recorded the movement of the mice between compartments. Mice were placed individually into the dark compartment at the beginning of the session. Total time spent in the light and dark compartments, the number of light to dark transitions, and total distance travelled during the 20 minute session were measured.

Elevated Plus-Maze (EPM).

EPM analysis was conducted using ANY-MAZE™ video tracking software as described in Sumislawski et al., 2011.

Rectal Temperature, Catalepsy, and Antinociception.

Mice were treated with either Compound A (10 mg/kg) or (R)-(+)-[2,3-Dihydro-5-methyl-3-(4-morpholinylmethyl)pyrrolo[1,2,3-de]-1,4-benzoxazin-6-yl]-1-naphthalenylmethanone (WIN-55,212-2; 10 mg/kg), or corresponding vehicle by i.p. injection. Every 15 minutes, the rectal temperature of the mice was taken using a lubricated rectal thermometer for a total of 1 hour post drug injection. To test catalepsy every 15 minutes the hindpaws of the mice were rested on a table and the front paws of the mouse were placed on a metal ring attached to a stand elevated 16 cm above the table. The time for the mouse to place its front paws on the table was recorded. For the hot plate antinociception test, mice were placed on a flat surface that was electrically-heated to 55° C. within an open Plexiglas tube, which was cleaned in between testing each mouse with Vimoba, a chlorine dioxide solution. The latency of the mice to respond upon placement on the hot plate apparatus by shaking, hindpaw licking, jumping, or tucking of the forepaws or hindpaws was recorded.

Novel Object Recognition.

Mice were handled 4 days prior to training for at least 1 min per day. During pre-training, mice were placed in an open Plexiglas rectangular chamber for 10 minutes in order for the mice to become familiar with the testing environment. Twenty-four hours later, the mice were placed into the same rectangular chamber with two identical sample objects, yellow rubber ducks, for 10 minutes in order for the mice to become familiar with the objects. During training, the sample objects were placed in opposite corners in the back of the chamber 5 cm from each wall and secured by weight to the floor of the chamber. Twenty-four hours later, the mice were placed into the chamber again with one sample or familiar object and a novel object, a white leaf statue, for 5 minutes. During testing, the objects were placed in opposite corners in the back of the chamber 5 cm from each wall and secured by weight to the floor of the chamber.

Two hours prior to testing, the mice were treated either with vehicle or Compound A (10 mg/kg), by i.p. injection. To determine exploration time with the sample and novel objects, each mouse was timed when interacting with the sample or novel object when the nose of the mouse was in contact with the object or directed toward the object within a 2 cm distance of the object. The time the mouse spent on top of the objects was not included in the exploration time analyses. In addition, a discrimination ratio (e.g., ratio of a mouse's interaction with a novel object to that mouse's total interaction with both sample and novel objects) was determined. If the discrimination ratio was above 0.5, it was considered that the mouse interacted more with the novel object than with the sample or familiar object.

Mass Spectrometry Analysis.

Analytes were quantified using LC-MS/MS on a Quantum triple-quadrupole mass spectrometer in positive-ion mode using selected reaction monitoring. Detection of eicosanoids was performed as described in Kingsley & Marnett, 2007. For fatty acid analysis the mobile phases used were 80 μM AgOAc with 0.1% (v/v) acetic acid in H₂O (solvent A) and 120 μM AgOAc with 0.1% (v/v) acetic acid in MeOH (solvent B). The analytes were eluted using a gradient from 20% A to 99% B over 5 minutes. The transitions used were m/z 300→282 for PEA, m/z 328→310 for SEA, m/z 434→416 for OEA, m/z 456→438 for AEA, m/z 464→446 for AEA-d8, m/z 331→257 for 2-PG, m/z 359→285 for 2-SG, m/z 463→389 for 2-OG, m/z 485→411 for 2-AG, m/z 493→419 for 2-AG-d8, m/z 519→409 for AA, and m/z 527→417 for AA-d8. Peak areas for the analytes were normalized to the appropriate internal standard and then normalized to tissue mass for in vivo samples.

Statistical Analysis.

Statistical analysis was performed using GRAPHPAD PRISM® Version 6.0c. For determining statistical significance between groups a two-tailed t-test, one-way ANOVA, or two-way ANOVA with a Sidak's post-test analysis, or multiple t-tests with Holm-Sidak a correction for multiple comparisons was used throughout. F and P values shown correspond to the value obtained from the test used. Error bars represent S.E.M. throughout. No treatment blinding was conducted. Sample sized were based on previous studies (see Patel et al., 2009). N for each group represents number of mice, i.e., independent biological replicate. Mice were arbitrarily assigned to treatment group in a manner that resulted in approximately equal sample sized per treatment group. Each treatment group was represented at least once per cage of mice.

### Example 11

### Development of Additional In Vivo Bioactive SSCIs

It has been shown that (R)-flurbiprofen, but not (S)-flurbiprofen, selectively inhibits eCB oxygenation by COX-2 in vitro without affecting AA oxygenation to PGs. This phenomenon was termed “substrate-selective COX-2 inhibition” (SSCI). However, (R)-flurbiprofen isomerizes to (S)-flurbiprofen in vivo (see Knihinicki et al., 1990) which limits its utility to probe the in vivo regulation of eCB signaling by SSCI.

To develop novel in vivo biologically active SSCIs, site-directed mutagenesis of COX-2 active site residues was employed to identify the key molecular interactions required for SSCI. Previous studies had established that mutations of Arg-120 and Tyr-355 of COX-2 drastically reduce the ability of the COX inhibitor indomethacin to inhibit AA oxygenation by eliminating its ability to ion-pair and hydrogen bond with COX-2 (Kalgutkar et al., 2000a). As expected, mutation of these key residues dramatically reduced the ability of indomethacin to inhibit AA oxygenation. However, it was determined that indomethacin still potently inhibited eCB oxygenation by the COX-2 R120Q and Y355F mutants. This indicated that, although ion-pairing and hydrogen-bonding with Arg-120 and Tyr-355 were critical for indomethacin inhibition of AA oxidation to PGs, they were much less important for inhibition of eCB oxygenation.

Whether structural modifications that reduced the ability of indomethacin to ion-pair with Arg-120 and Tyr-355 would lead to SSCI was also tested. A small library of tertiary amide derivatives of indomethacin, which are less polar and thus have reduced ion-pairing and hydrogen bonding capacity, was synthesized and screened. Each of the tertiary amides inhibited eCB oxygenation by COX-2 but did not inhibit AA oxygenation (Table 8).

TABLE 8
Analysis of Indomethacin and Primary, Secondary, and Tertiary Analogs

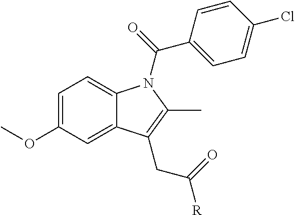

| Compound | R | AA IC₅₀ | | 2-AG IC₅₀ |
|---|---|---|---|---|
| indo- methacin | 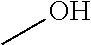 | 180 | nM | 30 nM |
| B | 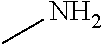 | 610 | nM | 590 nM |
| C | 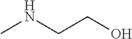 | 262 | nM | 237 nM |
| D | 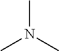 | 19 | μM | 424 nM |
| E | 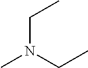 | 23 | μM | 583 nM |
| F | 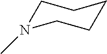 | >25 | μM | 653 nM |
| G | 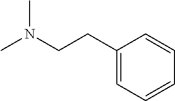 | >25 | μM | 715 nM |
| A | 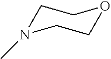 | >25 | μM | 622 nM |

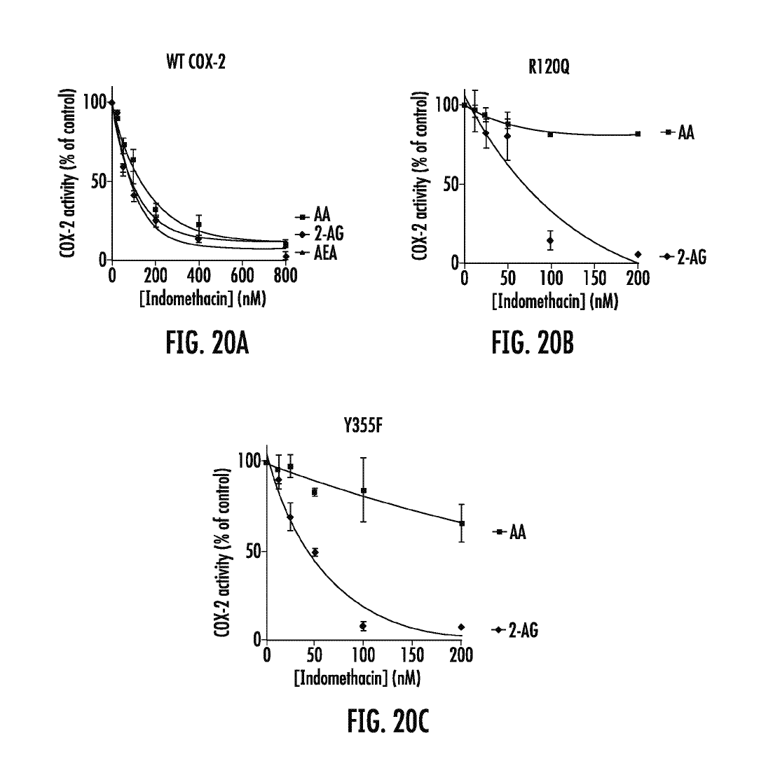

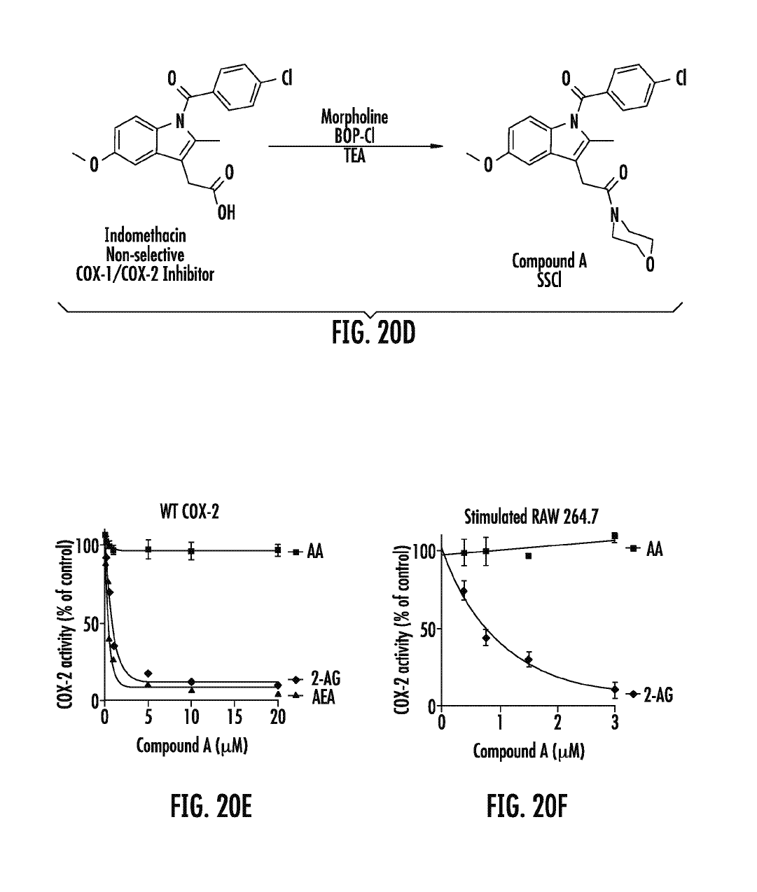

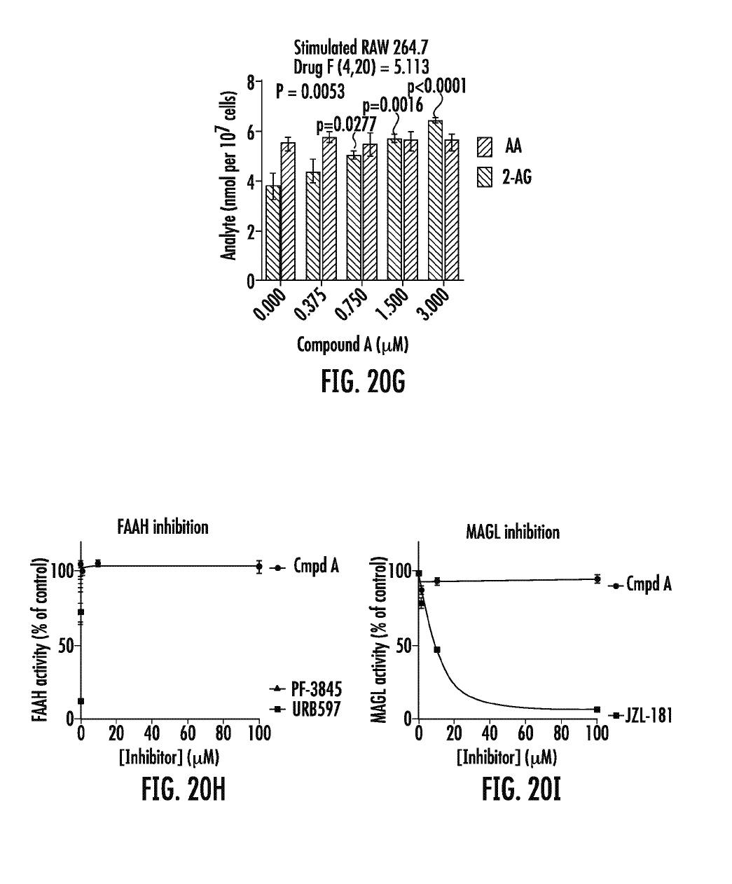

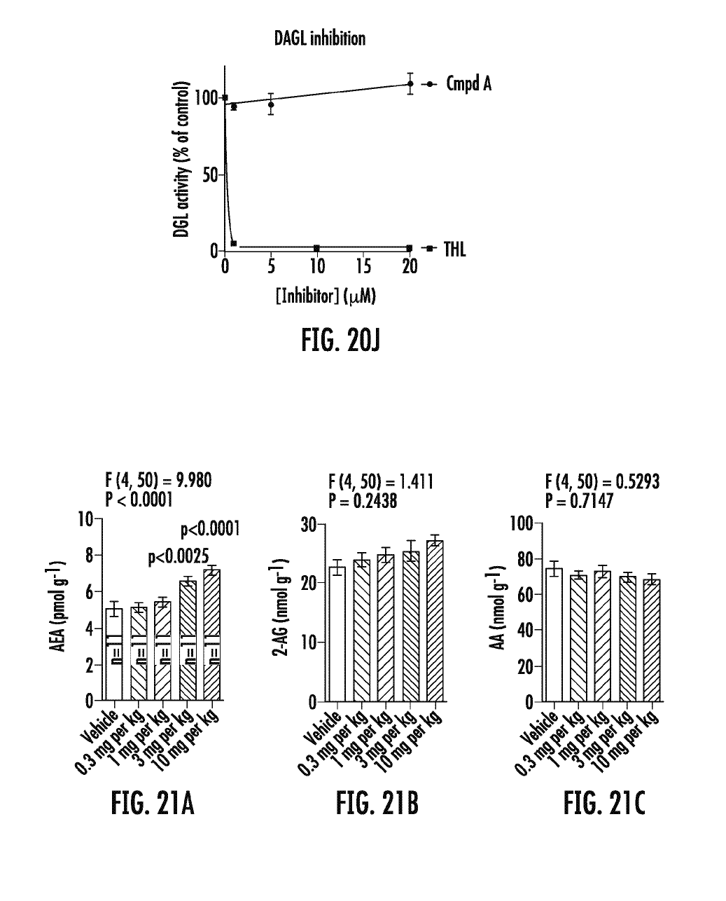

Compound A (FIG. 20d), was effective at inhibiting eCB oxygenation by purified COX-2 and by COX-2 in lipopolysaccharide-activated RAW 264.7 macrophages without inhibiting AA oxygenation (FIGS. 20e and 20f). Moreover, Compound A concentration-dependently increased 2-AG levels in stimulated RAW 264.7 macrophages without increasing AA levels, providing cellular evidence for substrate-selective pharmacology of Compound A (FIG. 20g). Importantly, Compound A did not inhibit other eCB metabolizing/synthetic enzymes including FAAH, MAGL, and DAGLα (FIGS. 20h-20j). Thus, Compound A exhibited multiple properties desirable in a SSCI, and was selected for subsequent in vivo studies.

### Example 12

### In Vivo Augmentation of eCB Levels by Compound A Via SSCI

To assess the ability of Compound A to modulate eCB levels in vivo, Compound A was adminstered to male ICR mice via intraperitoneal (i.p.) injection, and levels of eCBs, PGs, and AA were assayed two hours after administration. It was determined that Compound A significantly increased whole brain AEA levels at 3 mg/kg (p<0.01) and 10 mg/kg (p<0.0001) with a non-significant trend to increase 2-AG levels at the highest dose (FIGS. 21a and 21b). Compound A did not affect AA levels or PG levels at any dose tested (FIGS. 21c and 21d), demonstrating that the substrate-selectivity of Compound A was retained in vivo.

To more reliably quantify the magnitude of the increase in brain eCBs induced by Compound A treatment, a subsequent meta-analysis of data obtained from 12 cohorts of animals was conducted, normalizing eCB levels in each animal to mean eCB levels in the respective vehicle control group. This analysis revealed that, on average, Compound A at 10 mg/kg significantly increased AEA to 139% of vehicle (p<0.0001), and increased 2-AG levels to 109% of vehicle (p<0.01; FIGS. 21e and 21f). Analysis of brain extracts by LC-MS/MS revealed the presence of Compound A, but not indomethacin, after Compound A treatment (FIG. 22). Thus, Compound A selectively increased eCB levels without affecting AA or PG levels, and was present in the brain 2 hours after i.p. injection. To confirm that the in vivo substrate-selective profile of Compound A was unique relative to other COX inhibitors, the ability of indomethacin (10 mg/kg), a non-selective COX-1/COX-2 inhibitor and the parent compound of Compound A, the COX-2 selective inhibitor NS-398 (10 mg/kg), and the COX-1 selective inhibitor SC-560 (10 mg/kg), to modulate eCB, AA, and PG levels in vivo was determined. Compound A (p<0.0001), indomethacin (p<0.0001), NS-398 (p<0.001) and SC-560 (p<0.01) significantly increased AEA levels, while only Compound A (p<0.01) and indomethacin (p<0.01) significantly increased 2-AG levels (FIGS. 21g and 21h). Compound A did not significantly affect AA levels, while indomethacin (p<0.001), NS-398 (p<0.01), and SC-560 (p<0.01) all significantly increased AA levels (FIG. 21i). Indomethacin, NS-398, and SC-560 (p<0.0001 for all), but not Compound A, profoundly decreased brain PG levels (FIG. 21j). These data indicated that in vivo substrate-selective pharmacological profile of Compound A was unique, and not shared by traditional COX inhibitors.

COX-2 was then confirmed as the in vivo molecular target mediating the increase in brain eCBs observed after Compound A treatment using COX-2 knockout (ptgs-2^{(-/-)}; COX-2^{(-/-)}) mice. Compound A (10 mg/kg) significantly increased AEA (p<0.01) and 2-AG (p<0.05) levels in wild type, but not COX-2^{(-/-)} littermates (FIGS. 21k and 21l). Importantly, COX-2^{(-/-)} mice had significantly higher brain AEA levels than WT littermates at baseline (p<0.0001). Lastly, Compound A did not affect AA or PG levels in WT or COX-2^{(-/-)} mice (FIGS. 21m and 21n). These data confirmed that Compound A increased brain eCB levels via a COX-2 dependent mechanism, and COX-2 substantially regulated basal brain AEA levels in vivo.

### Example 13

### Compound a Selectively Increases eCBs Over Structurally-Related Non-eCB Lipids

One major limitation of currently available eCB degradation inhibitors is their lack of selectivity for eCBs over related non-eCB lipids. For example, FAAH inhibition increases AEA levels, but also increases levels of a class of N-acylethanolamides (NAEs; oleoylethanolamide (OEA), palmitoylethanolamide (PEA), and stearoylethanolamide (SEA)). Similarly MAGL inhibition increases 2-AG levels, but also a class of related monoacylglycerols (MAGs) including 2-oleoylglycerol (2-OG), 2-palmitoylglycerol (2-PG), and 2-stearoylglycerol (2-SG).

Given the selectivity of COX-2 for AA-containing lipids, whether Compound A would be more selective for the AA containing NAE, AEA, and the AA containing MAG, 2-AG, relative to other non-eCB members of the NAE and MAG lipid classes was examined. Targeted lipid profiling of NAEs and MAGs after Compound A treatment was employed. As expected, Compound A significantly increased brain AEA (p<0.0001; FIG. 23a), but not any other NAE. In contrast, the FAAH inhibitor PF-3845 (Long et al., 2009a; Kinsey et al., 2010) at 10 mg/kg robustly increased all NAEs including AEA (FIG. 23b). These data strongly suggested that Compound A selectively increased brain AEA over other NAEs, and that its mechanism of action was not via off target FAAH inhibition, since if this were the case one would also have expected to see increases in other NAEs.

To further exclude FAAH inhibition as contributing to the AEA elevating effects of Compound A, several additional experiments were conducted. First, it was determined that Compound A caused a significant additional increase in AEA levels (p<0.01) when combined with the FAAH inhibitor PF-3 845, compared to PF-3845 treatment alone (FIG. 23c), suggesting different mechanisms of action between the FAAH inhibitor and Compound A.

Second, if Compound A increased AEA via FAAH inhibition, one would expect an increase in NAEs in tissues with very high FAAH expression such as the liver. However, it was determined that Compound A did not affect levels of any NAE in the liver, while PF-3845 caused robust increases in levels of all NAEs in this FAAH rich tissue (FIG. 23d).

Lastly, it was determined that Compound A increased brain AEA levels in WT and FAAH KO mice to a similar extent (FIG. 23e). Taken together with the biochemical data presented herein that Compound A does not inhibit FAAH activity in vitro, these converging in vivo data strongly suggested a unique COX-2 mediated mechanism of action of Compound A to increase AEA levels.

The selectivity of Compound A for 2-AG over other MAGs as compared to the MAGL inhibitor JZL-184 (40 mg/kg) was also tested. While Compound A (10 mg/kg) significantly increased brain 2-AG levels (p<0.05), it did not affect levels of any other MAG (FIG. 23f). In contrast, the MAGL inhibitor JZL-184 increased levels of 2-AG and 3 other MAG species (FIG. 23g). Furthermore, Compound A produced an additional significant increase in 2-AG levels after JZL-184 treatment (p<0.05) compared to JZL-184 alone (FIG. 23h). Combined with the in vitro data presented herein that Compound A did not affect MAGL activity, these in vivo data strongly suggested that the ability of Compound A to increase 2-AG levels was not mediated via MAGL inhibition.

Additionally, the selectivity of the regulation of basal brain eCBs by COX-2 over related non-eCB NAEs was also determined. To that end, the effect of Compound A on brain NAEs in WT and COX-2^{(-/-)} mice was examined. It was again found that Compound A significantly increased brain AEA (p<0.001), but no other NAE (FIG. 23h), and importantly, COX-2^{(-/-)} mice had increased levels of only AEA (p<0.001), but no other NAE. These data confirmed a key role for COX-2 in the selective regulation of basal AEA over other non-eCB NAEs.

### Example 14

### Compound A Increased Peripheral eCB Levels

Like other eCB degrading enzymes including FAAH and MAGL, COX-2 is expressed in many tissues. The effects of Compound A on eCB, NAE, MAG, and PG levels in a variety of peripheral tissues was tested. Compound A (10 mg/kg) significantly increased AEA levels in the stomach (p<0.05), small intestine (p<0.05), kidney (p<0.0001), and lung (p<0.001) but not heart (FIGS. 24a-24e) or liver (FIG. 23d). In contrast, Compound A did not affect levels of any other NAE in any tissue. Similarly, Compound A had no effect on 2-AG or any other MAG in any tissue examined (FIGS. 24a-24e).

To confirm that Compound A retained substrate-selective pharmacology outside the CNS, levels of PGs in each tissue after Compound A and indomethacin treatment was tested. As expected, indomethacin robustly decreased PGs in all tissues examined (p<0.0001 for stomach, heart, kidney and lung; p<0.001 for intestine), while Compound A had no effect on PGs levels in any tissue examined (FIGS. 24a-24e). These data indicated that SSCI can selectively augment AEA levels without affecting non-eCB NAEs in most peripheral tissues, and that Compound A retains in vivo substrate-selectivity in peripheral tissues.

### Example 15

### Compound A Reduces Anxiety Via eCB Augmentation

Since eCB augmentation via FAAH inhibition has been suggested to represent a novel approach to the treatment of mood and anxiety disorders (Kathuria et al., 2003; Piomelli, 2005)), the behavioral effects of Compound A in pre-clinical models of anxiety were tested. FAAH inhibitors had been reported to have anxiolytic activity in the novel open-field arena (Rossi et al., 2010), and in agreement with this, it was determined that the FAAH inhibitor PF-3 845 (10 mg/kg) increased center distance travelled and center time in the open-field arena (FIG. 25a). Importantly, Compound A (10 mg/kg) also increased center distance traveled and center time (FIG. 25b), suggesting it had similar behavioral effects as a FAAH inhibitor with respect to reducing anxiety in this assay.

To test the hypothesis that all COX inhibitors that increase brain AEA exert a similar behavioral profile to Compound A, the effect of indomethacin (10 mg/kg), NS-398 (10 mg/kg), and SC-560 (10 mg/kg) were also tested in the open-field arena (Supplemental FIGS. 21a and 21b). Indomethacin and NS-398, which both increased brain AEA levels, both increased center time and center distance traveled in the open-field arena, suggestive of an anxiolytic behavioral effect. However, SC-560, which had only marginal effects on brain AEA levels, did not have any significant behavioral effect in this assay (FIG. 26c).

To determine if the behavioral effects of Compound A were mediated by substrate-selective inhibition of COX-2, the behavioral effects of Compound A were tested in COX-2^{(-/-)} mice and wild type littermates. While exhibiting a normal anxiolytic-like response in wild type littermates (FIGS. 25c-25e), Compound A did not produce any significant behavioral effects in COX-2^{(-/-)} mice (FIG. 25c-25e), in accordance with the lack of change in AEA levels seen after Compound A administration to COX-2^{(-/-)} mice. Interestingly, COX-2^{(-/-)} mice showed a slight anxiolytic phenotype relative to wild type littermates on center time spent in the open field (FIG. 27).

Since the anxiolytic effects of FAAH inhibition are mediated via AEA acting on CB1 cannabinoid receptors (Kathuria et al., 2003; Moreira et al., 2008), the role of CB1 receptors in the anxiolytic-like behavioral effects of Compound A in the open field arena was tested. While again exerting anxiolytic actions in vehicle-pretreated mice, Compound A had no significant behavioral effect in mice treated with the CB1 receptor antagonist Rimonabant (5-(4-Chlorophenyl)-1-(2,4-dichloro-phenyl)-4-methyl-N-(piperidin-1-yl)-1H-pyrazole-3-carboxamide) at 3 mg/kg (FIGS. 5f-5h). Furthermore, Compound A did not produce any behavioral effect in CB1^{(-/-)} mice (FIG. 28a).

To verify that Compound A actually increased eCB levels in CB1^{(-/-)} mice, the eCB levels and PG levels in CB1^{(-/-)} mice after Compound A treatment was analzyed, and it was determined that Compound A increased AEA and 2-AG levels in the brains of the CB1^{(-/-)} mice while again having no effect on PG levels (FIG. 28b). These data suggested that the behavioral effects of Compound A were mediated via SSCI activity, which selectively increased brain eCB levels leading to increased activation of central CB 1 receptors and anxiolytic behavioral effects.

To further assess the anxiolytic-like effects of eCB augmentation by Compound A, the effects of Compound A and the FAAH inhibitor PF-3845 in the light-dark box and the elevated plus-maze were analyzed. In the light-dark box test, it was determined that the FAAH inhibitor PF-3845 (10 mg/kg) and Compound A (10 mg/kg) both significantly increased light-zone time (p<0.05 for both drugs) and the number of light-zone entries (p<0.001 PF-3845, and p<0.01 Compound A) without significantly affecting overall locomotor activity, further validating the anxiolytic-like actions of Compound A (FIGS. 29a-29f). Importantly, the anxiolytic effects of Compound A were again blocked by the CB1 receptor antagonist Rimonabant (3 mg/kg), indicating that the observed behavioral effects were mediated via CB1 receptor activation (FIGS. 29d-29f).

In the elevated plus-maze both PF-3845 (10 mg/kg) and Compound A (10 mg/kg) significantly reduced open arm latency (p<0.01 for PF-3845 and p<0.0001 for Compound A; FIG. 30), but did not affect other parameters including total distance traveled. Overall, the effects of Compound A closely mirrored the behavioral profile of the FAAH inhibitor PF-3845 in all anxiety measures examined, suggesting that SSCI exerted anxiolytic actions via eCB augmentation in a similar manner to FAAH inhibition. These studies provided proof-of-concept validation that SSCIs represent a novel class of COX-2-based anxiolytic agents that exert anxiolytic actions via eCB activation.

### Example 16

### Compound A Lacks Overt Cannabimimetic Activity In Vivo

Finally, the relative cannabimimetic effects of Compound A was examined in vivo. Exogenous activation of CB1 receptors produces a classical “tetrad” of behavioral effects characterized by hypolocomotion, analgesia, catalepsy, and hypothermia (Fride et al., 2006). The open field data indicated that Compound A did not cause hypolocomotion, and additional studies revealed that Compound A did not cause hypothermia, catalepsy, or antinociception in the hot plate test (FIGS. 31a-31c). In contrast, the CB1 agonist Win-55212-2 induced hypothermia, catalepsy and anti-nociception (FIGS. 31a-31c). Compound A also did not induce memory deficits in the novel object recognition assay when administered prior to object memory retrieval (FIG. 31d). Compound A was also shown not to cause gastrointestinal hemorrhage, a primary adverse effect of COX-1/2 inhibitors including indomethacin, the parent drug of Compound A. Taken together, these data indicated that Compound A induced a subset of behavioral effects mediated via eCB activation, but did not cause overt cannabimimetic effects, and also did not cause overt GI toxicity observed with many traditional COX inhibitors including indomethacin.

### Discussion of EXAMPLES 11-16

Pharmacological approaches to augment eCB signaling have thus far focused on inhibition of FAAH and MAGL. Both approaches have been validated to robustly augment eCB levels in vivo (Kathuria et al., 2003; Long et al., 2009b), and exert preclinical therapeutic effects in a variety of pathological conditions. However, both approaches increase non-eCB lipids (NAEs for FAAH inhibition and MAGs for MAGL inhibition) that have biological actions at targets other than cannabinoid receptors (O'Sullivan, 2007). Disclosed herein are investigations that demonstrated that COX-2 is a key regulator of eCB levels in vivo, and that substrate-selective inhibition of COX-2 represents a viable alternative approach to augment eCB levels with a high degree of selectivity (FIG. 32). In contrast to traditional COX inhibitors, the SSCI Compound A increased AEA without affecting central or peripheral PGs.

These data validated the in vivo substrate-selectivity of Compound A and provide a novel and effective pharmacological strategy to selectively augment central eCB signaling via COX-2 inhibition. The larger effects of COX-2 inhibition on AEA over 2-AG might be related to the closer proximity between COX-2 and the site of AEA biosynthesis, and might explain its relative lack of overt cannabimimetic effects (Long et al., 2009a). In support of this notion, FAAH, which is the primary metabolic regulator of AEA, is localized to the same postsynaptic cellular compartment as COX-2 (Cristino et al., 2008).

Furthermore, Compound A did not affect levels of non-eCB NAEs or MAGs in the brain or periphery, providing enhanced selectivity over FAAH and MAGL inhibition for eCB augmentation. That Compound A was able to increase AEA in several peripheral tissues tested suggested that COX-2 plays a widespread role in the regulation of AEA signaling. Although the precise mechanisms regulating tissue specificity of Compound A remain to be determined, they likely relate to relative levels of COX-2 and FAAH, and rates of tonic AEA biosynthesis in each tissue.

Consistent with studies demonstrating that elevating AEA levels via FAAH inhibition exerts anxiolytic-like actions in preclinical models (Kathuria, et al., 2003; Patel & Hillard, 2006; Moreira et al., 2008; Rossi et al., 2010), it was also determined that Compound A decreased anxiety-like behaviors using multiple validated assays. These data suggested that SSCIs could represent a viable approach to the treatment of mood and anxiety disorders. Interestingly, COX-2 inhibition has demonstrated clinical antidepressant efficacy as an adjunct to traditional antidepressants (Muller & Schwarz, 2008). The data presented herein raised the intriguing possibility that these effects could be in part due to augmentation of brain eCB signaling.

In addition to having beneficial effects in the brain, the substrate-selectivity of Compound A could potentially be employed to reduce some common side effects mediated by inhibition of PG synthesis by traditional NSAIDs. Gastrointestinal PG production is essential for stimulation of mucosal bicarbonate and mucus secretion as well as increasing mucosal blood flow (Patrignani et al., 2011). As such, traditional NSAIDs are associated with serious gastrointestinal complications. The data presented herein indicated that an exemplary SSCI, Compound A, did not cause overt gastrointestinal hemorrhage as seen with indomethacin.

Furthermore, cardiovascular toxicity problems of COX inhibitors are well-established, and have been suggested to be mediated by inhibition of PG synthesis (Yu et al., 2012). As such, SSCI might be devoid of such toxicity since they do not affect PG levels in the heart or lung.

Disclosed herein are experiments demonstrating that COX-2 is a key regulator of brain eCB signaling in vivo and that substrate-selective inhibition of COX-2 could represent a novel pharmacological approach to the treatment of anxiety disorders. Moreover, given the numerous pathological processes in which dysregulation of eCB signaling has been demonstrated coupled with the high degree of selectivity of SSCIs for eCBs over related lipids, SSCIs represent a novel class of pharmaceutical agents with broad therapeutic potential.

### Example 17

### Inhibition of mCOX-2-Dependent Oxygenation of 2-AG and AA by Lumiracoxib and Derivatives In Vitro^{x}

Lumiracoxib and certain derivatives thereof were synthesized and tested for their abilities to inhibit mCOX-2-dependent oxygenation of 2-AG and AA in vitro.

IC₅₀ values were determined by incubating five concentrations of each inhibitor and a solvent control in DMSO with purified murine COX-2 (50 nM) for three minutes, followed by addition of 2-AG or AA (5 mM) at 37° C. for 30 seconds. The structures of the derivatives and the IC₅₀ values determined are summarized in Table 9.

TABLE 9
Inhibition of mCOX-2-Dependent Oxygenation of 2-AG and AA by
Lumiracoxib and Derivatives In Vitro^{x}

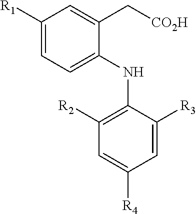

| Inhibitor | R₁ | R₂ | R₃ | R₄ | 2-AG IC₅₀ (mM)^{y} | AA IC₅₀ (mM)^{z} |
|---|---|---|---|---|---|---|
| 101 | CH₃ | F | Cl | H | 0.04 ± 0.01 | -(25%) |
| 102 | CH₃ | H | Cl | H | 0.06 ± 0.01 | -(0%) |
| 103 | CH₃ | F | H | H | 1.8 ± 0.6 | -(0%) |
| 104 | CH₃ | H | H | H | -(0%) | -(10%) |
| 105 | CH₃ | Cl | Cl | H | 0.03 ± 0.01 | 0.2 ± 0.1 (60%) |
| 106 | CH₃ | F | F | H | 1.0 ± 0.3 | -(0%) |
| 107 | CH₃ | F | CH₃ | H | -(0%) | -(0%) |
| 108 | CH₃ | Cl | Cl | Cl | 0.07 ± 0.01 | -(32%) |
| 109 | H | F | Cl | H | 0.04 ± 0.01 | -(15%) |
| 110 | H | H | Cl | H | 3.9 ± 2.0 | -(0%) |
| 111 | H | F | H | H | -(25%) | -(0%) |
| 112 | H | H | H | H | -(8%) | -(15%) |
| 113^{c} | H | Cl | Cl | H | 0.03 ± 0.01 | 0.1 ± 0.01 (85%) |
| 114 | H | F | F | H | 1.2 ± 0.4 | -(10%) |
| 115 | H | F | CH₃ | H | -(15%) | -(0%) |
| 116 | H | Cl | Cl | Br | 0.06 ± 0.02 | 0.14 ± 0.05 (55%) |

^{y}Mean ± standard deviation (n = 6); dash (-) indicates <50% inhibition of 2-AG oxygenation at 10 mM inhibitor. Numbers in parentheses indicate maximum inhibition (when not equal to 100%) at 10 mM inhibitor.
^{z}Inhibitor incubated with enzyme for 15 min before addition of 2-AG or AA.

The data presented in Table 9 demonstrated that in some embodiments, substrate-selective inhibition in the diclofenac/lumiracoxib series required at least one halogen in the anilino ring. For those compounds with only a single halogen in the anilino ring in the diclofenac series that were weak substrate-selective inhibitors, potency could be increased by adding a methyl group in the meta position of the phenylacetic acid ring, thereby making them members of the lumiracoxib series.

### REFERENCES

All references listed in the instant disclosure, including but not limited to all patents, patent applications and publications thereof, scientific journal articles, and database entries (including but not limited to GENBANK® database entries and including all annotations available therein) are incorporated herein by reference in their entireties to the extent that they supplement, explain, provide a background for, and/or teach methodology, techniques, and/or compositions employed herein.
Adams et al. (2002) PHENIX: building new software for automated crystallographic structure determination. Acta Crystallogr D Biol Crystallogr 58:1948-1954.
Adams et al. (2010) PHENIX: a comprehensive Python-based system for macromolecular structure solution. Acta Cryst D66:213-221.
Ahn et al. (2008) Enzymatic pathways that regulate endocannabinoid signaling in the nervous system. Chem Rev 108:1687-1707.
Ahn et al. (2009) Discovery and Characterization of a Highly Selective FAAH Inhibitor that Reduces Inflammatory Pain. Chem Biol 16:411-420.
Allison et al. (1992) Gastrointestinal Damage Associated with the Use of Nonsteroidal Antiinflammatory Drugs. N Engl J Med 327:749-754.
Berkow et al. (1997) The Merck Manual of Medical Information, Home ed., Merck Research Laboratories, Whitehouse Station, New Jersey.
Bhattacharyya et al. (1996) Involvement of arginine 120, glutamate 524, and tyrosine 355 in the binding of arachidonate and 2-phenylpropionic acid inhibitors to the cyclooxygenase active site of ovine prostaglandin endoperoxide H synthase-1. J Biol Chem 271:2179-2184.
Bifulco et al. (2007) Endocannabinoids as emerging suppressors of angiogenesis and tumor invasion (review). Oncol Rep 17:813-816.
Bishay et al. (2010) R-flurbiprofen reduces neuropathic pain in rodents by restoring endogenous cannabinoids. PLoS ONE 5:e10628.
Bisogno & Di Marzo (2010) Cannabinoid receptors and endocannabinoids: role in neuroinflammatory and neurodegenerative disorders. CNS Neurol Disorders Drug Targets 9:564-573.
Blankman et al. (2007) A comprehensive profile of brain enzymes that hydrolyze the endocannabinoid 2-arachidonoylglycerol. Chem Biol 14:1347-1356.
Blobaum & Marnett (2007) Molecular Determinants for the Selective Inhibition of Cyclooxygenase-2 by Lumiracoxib. J Biol Chem 282:16379-16390.
Brebion et al. (2003) Highly Diastereoselective Conjugate Addition to Alkylidene Bis(Sulfoxides): Asymmetric Synthesis of (+)-erythro-Roccellic Acid. Angewandte Chemie Intl Ed 42:5342-5345.
Brune et al. (1992) Pure enantiomers of 2-arylpropionic acids: tools in pain research and improved drugs in rheumatology. J Clin Pharmacol 32:944-952.
Centonze et al. (2007) The endocannabinoid system in targeting inflammatory neurodegenerative diseases. Trends Pharmacol Sci 28:180-187.
Chen et al. (2008) Identification of novel endogenous cytochrome P450 arachidonate metabolites with high affinity for cannabinoid receptors. J Biol Chem 283:24514-24524.
Cravatt & Lichtman (2003) Fatty acid amide hydrolase: an emerging therapeutic target in the endocannabinoid system. Curr Opin Chem Biol 7:469-475.
Cravatt et al. (1996) Molecular characterization of an enzyme that degrades neuromodulatory fatty-acid amides. Nature 384:83-87.
Cristino et al. (2008) Immunohistochemical localization of anabolic and catabolic enzymes for anandamide and other putative endovanilloids in the hippocampus and cerebellar cortex of the mouse brain. Neuroscience 151:955-968.
Davie et al. (1982) A Double, Blind Comparison of Parenteral Morphine, Placebo, and Oral Fenoprofen in Management of Postoperative Pain. Anesthesia & Analgesia 61:1002-1005.
DeWitt & Smith (1988) Primary Structure of Prostaglandin G/H Synthase from Sheep Vesicular Gland Determined from the Complementary DNA Sequence. Proc Natl Acad Sci USA 85:1412-1416.
Di Marzo (2008) Endocannabinoids: synthesis and degradation. Rev Physiolo Biochem Pharmacol 160:1-24.
Di Marzo (2009) The endocannabinoid system: its general strategy of action, tools for its pharmacological manipulation and potential therapeutic exploitation. Pharmaco Res 60:77-84.
Di Marzo et al. (2005) The biosynthesis, fate and pharmacological properties of endocannabinoids. Handb Exp Pharmacol 168:147-185.
Di Marzo et al. (2011) Cannabinoids and endocannabinoids in metabolic disorders with focus on diabetes. Handb Exp Pharmacol 203:75-104.
Dinh et al. (2004) RNA interference suggests a primary role for monoacylglycerol lipase in the degradation of the endocannabinoid 2-arachidonoylglycerol. Mol Pharmacol 66:1260-1264.
Dong et al. (2011) Human cyclooxygenase-2 is a sequence homodimer that functions as a conformational heterodimer. J Biol Chem 286:19035-19046.
Duch et al. (1998) Volatile anesthetics significantly suppress central and peripheral mammalian sodium channels. Toxicol Lett 100-101:255-263.
Duggan et al. (2010) Molecular basis for cyclooxygenase inhibition by the non-steroidal anti-inflammatory drug, naproxen. J Biol Chem 285:34950-34959.
Duggan et al. (2011). (R)-Profens are substrate-selective inhibitors of endocannabinoid oxygenation by COX-2. Nat Chem Biol 7:803-809
Ebadi (1998) CRC Desk Reference of Clinical Pharmacology. CRC Press, Boca Raton, Fla.
Emsley & Cowtan (2004) Coot: model-building tools for molecular graphics. Acta Crystallogr D Biol Crystallogr 60:2126-2132.
Emsley et al. (2010) Features and development of Coot. Acta Cryst D66:486-501.
Fowler et al. (2010) Modulation of the endocannabinoid system: Neuroprotection or neurotoxicity?Exper Neurol 224:37-47.
Freireich et al. (1966) Quantitative comparison of toxicity of anticancer agents in mouse, rat, hamster, dog, monkey, and man. Cancer Chemother Rep 50:219-244.
Fride et al. (2006) Behavioral methods in cannabinoid research. Methods Mol Med 123: 269-290.
Funk (2001) Prostaglandins and Leukotrienes: Advances in Eicosanoid Biology. Science 298:1871-1875.
Gaetani et al. (2009) The endocannabinoid system as a target for novel anxiolytic and antidepressant drugs. Intl Rev Neurobiol 85:57-72.
Garavito & Mulichak (2003) The structure of mammalian cyclooxygenases. Annu Rev Biophys Biomol Struct 32:183-206.
Gerbino (2005) Remington: The Science and Practice of Pharmacy. 21st Edition. Lippincott Williams & Wilkins, Philadelphia, Pa.
Glaser & Kaczocha (2010) Cyclooxygenase-2 mediates anandamide metabolism in the mouse brain. J Pharmacol Exp Ther 335:380-388.
Goodman et al. (1996) Goodman & Gilman's the Pharmacological Basis of Therapeutics, 9th ed., McGraw-Hill Health Professions Division, New York.
Guay et al. (2004) Carrageenan-induced paw edema in rat elicits a predominant prostaglandin E2 (PGE2) response in the central nervous system associated with the induction of microsomal PGE2 synthase-1. J Biol Chem 279:24866-24872.
Guindon & Hohmann (2009) The endocannabinoid system and pain. CNS Neurol Disord Drug Targets 8:403-421.
Hla & Neilson (1992) Human cyclooxygenase-2 cDNA. Proc Natl Acad Sci USA 89:7384-7388.
Hohmann & Suplita (2006) Endocannabinoid mechanisms of pain modulation. AAPS J 8:E693-708.
Hu et al. (2008) Prostaglandin E2 glycerol ester, an endogenous COX-2 metabolite of 2-arachidonoylglycerol, induces hyperalgesia and modulates NFkappaB activity. Br J Pharmacol 153:1538-1549.
Jamali et al. (1988) A.S. Stereoselective pharmacokinetics of flurbiprofen in humans and rats. J Pharm Sci 77:666-669.
Kalgutkar et al. (1998) Covalent modification of cyclooxygenase-2 (COX-2) by 2-(acetoxyphenyl)alkyl sulfides, a new class of selective COX-2 inactivators. J Med Chem 41:4800-4818.
Kalgutkar et al. (2000a) Biochemically based design of cyclooxygenase-2 (COX-2) inhibitors: facile conversion of nonsteroidal antiinflammatory drugs to potent and highly selective COX-2 inhibitors. Proc Natl Acad Sci USA 97:925-930.
Kalgutkar et al. (2000b) Ester and amide derivatives of the nonsteroidal antiinflammatory drug, indomethacin, as selective cyclooxygenase-2 inhibitors. J Med Chem 43:2860-2870.
Kathuria et al. (2003) Modulation of anxiety through blockade of anandamide hydrolysis. Nature Med 9:76-81.
Katzung (2001) Basic & Clinical Pharmacology, 8th ed., Lange Medical Books/McGraw-Hill Medical Pub. Division, New York.
Kim & Alger (2004) Inhibition of cyclooxygenase-2 potentiates retrograde endocannabinoid effects in hippocampus. Nature Neurosci 7:697-698.
Kingsley & Marnett (2007) LC-MS-MS analysis of neutral eicosanoids. Methods Enzymol 433:91-112.
Kinsey et al. (2010) Inhibition of endocannabinoid catabolic enzymes elicits anxiolytic-like effects in the marble burying assay. Pharmacol Biochem Behav 98:21-27.
Knihinicki et al. (1990) Stereoselective disposition of ibuprofen and flurbiprofen in rats. Chirality 2:134-140.
Kogan & Mechoulam (2006) The chemistry of endocannabinoids. J Endocrinol Invest 29:3-14.
Kozak et al. (2000) Oxygenation of the endocannabinoid, 2-arachidonylglycerol, to glyceryl prostaglandins by cyclooxygenase-2. J Biol Chem 275:33744-33749.
Kozak et al. (2001) Metabolism of Prostaglandin Glycerol Esters and Prostaglandin Ethanolamides in Vitro and in Vivo. J Biol Chem 276:36993-36998.
Kujubu et al. (1991) TIS10, A Phorbol Ester Tumor Promoter Inducible mRNA from Swiss 3T3 Cells, Encodes a Novel Prostaglandin Synthase/Cyclooxygenase Homologue. J Biol Chem 266:12866-12872.
Kulmacz & Lands (1985) Stoichiometry and kinetics of the interaction of prostaglandin H synthase with anti-inflammatory agents. J Biol Chem 260:12572-12578
Kunos (2007) Understanding metabolic homeostasis and imbalance: what is the role of the endocannabinoid system? Am J Med 120:S18-24; discussion S24.
Kurumbail et al. (1996) Structural basis for selective inhibition of cyclooxygenase-2 by anti-inflammatory agents. Nature 384:644-648.
Laskowski et al. (1993) PROCHECK: a program to check the stereochemical quality of protein structures, J Appl Cryst 26:283-291.
Lee et al. (1992) Selective Expression of Mitogen-Inducible Cyclooxygenase in Macrophages Stimulated with Lipopolysaccharide. J Biol Chem 267:25934-25938.
Leipold et al. (2004) Bioinversion of R-flurbiprofen to S-flurbiprofen at various dose levels in rat, mouse, and monkey. Chirality 16:379-387.
Liang et al. (2008) Identification and pharmacological characterization of the prostaglandin FP receptor and FP receptor variant complexes. Br J Pharmacol 154:1079-1093.
Lichtman et al. (2002) Endocannabinoids in cognition and dependence. Prostaglandins Leukot Essent Fatty Acids 66:269-285.
Long et al. (2009a) Dual blockade of FAAH and MAGL identifies behavioral processes regulated by endocannabinoid crosstalk in vivo. Proc Natl Acad Sci USA 106:20270-20275.
Long et al. (2009b) Selective blockade of 2-arachidonoylglycerol hydrolysis produces cannabinoid behavioral effects. Nat Chem Biol 5:37-44.
Lopez-Rodriguez et al. (2005) Involvement of cannabinoids in cellular proliferation. Mini Rev Med Chem 5:97-106.
Lötsch et al. (1995) Effects of flurbiprofen enantiomers on pain-related chemo-somatosensory evoked potentials in human subjects. Br J Clin Pharmacol 40:339-346.
Lutz (2009) Endocannabinoid signals in the control of emotion. Curr Opin Pharmacol 9:46-52.
Makriyannis et al. (2005) Therapeutic opportunities through modulation of the endocannabinoid system. Neuropharmacol 48:1068-1071.
Mangieri & Piomelli (2007) Enhancement of endocannabinoid signaling and the pharmacotherapy of depression. Pharmacological research: Off J Italian Pharmacol Soc 56:360-366.
Meade et al. (1993) Differential inhibition of prostaglandin endoperoxide synthase (cyclooxygenase) isozymes by aspirin and other non-steroidal anti-inflammatory drugs. J Biol Chem 268:6610-6614.
Monti et al. (2005) Binding of a chiral drug to a protein: an investigation of the 2-(3-benzoylphenyl)propionic acid/bovine serum albumin system by circular dichroism and fluorescence. Phys Chem Chem Phys 7:4002-4008.
Moreira et al. (2008) Reduced anxiety-like behaviour induced by genetic and pharmacological inhibition of the endocannabinoiddegrading enzyme fatty acid amide hydrolase (FAAH) is mediated by CB1 receptors. Neuropharmacol 54:141-150.
Muller & Schwarz (2008) COX-2 inhibition in schizophrenia and major depression. Curr Pharm Des 14:1452-1465.
Naidu et al. (2007) Evaluation of fatty acid amide hydrolase inhibition in murine models of emotionality. Psychopharmacol 192:61-70.
Nirodi et al. (2004) The glyceryl ester of prostaglandin E2 mobilizes calcium and activates signal transduction in RAW264.7 cells. Proc Natl Acad Sci USA 101:1840-1845.
Nomura et al. (2011) Endocannabinoid Hydrolysis Generates Brain Prostaglandins That Promote Neuroinflammation. Science 334:809-813.
O'Sullivan (2007) Cannabinoids go nuclear: evidence for activation of peroxisome proliferator-activated receptors. Br J Pharmacol 152:576-582.
O'Sullivan et al. (1993) Lipopolysaccharide-lnduced Expression of Prostaglandin H Synthase-2 in Alveolar Macrophages is Inhibited by Dexamethasone by not by Aspirin. Biochem Biophys Res Commun 191:1294-1300.
Ossipov et al. (2000) Differential effects of spinal (R)-ketoprofen and (S)-ketoprofen against signs of neuropathic pain and tonic nociception: evidence for a novel mechanism of action of (R)-ketoprofen against tactile allodynia. Pain 87:193-199.
Otwinowski & Minor (1997) Processing of X-ray diffraction data collected in oscillation mode. Methods Enzymol 276A:307-326.
Pan et al. (2008) D-2 Dopamine Receptor Activation Facilitates Endocannabinoid-Mediated Long-Term Synaptic Depression of GABAergic Synaptic Transmission in Midbrain Dopamine Neurons via cAMP-Protein Kinase A Signaling. J Neurosci 28:14018-14030.
Patel & Hillard (2006) Pharmacological evaluation of cannabinoid receptor ligands in a mouse model of anxiety: further evidence for an anxiolytic role for endogenous cannabinoid signaling. J Pharmacol Exp Ther 318:304-311.
Patel & Hillard (2008) Adaptations in endocannabinoid signaling in response to repeated homotypic stress: a novel mechanism for stress habituation. Eur J Neurosci 27:2821-2829.
Patel et al. (2009) Repeated Homotypic Stress Elevates 2-Arachidonoylglycerol Levels and Enhances Short-Term Endocannabinoid Signaling at Inhibitory Synapses in Basolateral Amygdala. Neuropsychopharmacol 34:2699-2709.
Patrignani et al. (2011) Managing the adverse effects of nonsteroidal anti-inflammatory drugs. Expert Rev Clin Pharmacol 4:605-621.
PCT International Patent Application Publication No. WO 93/25521.
Pedicord et al. (2011) Molecular characterization and identification of surrogate substrates for diacylglycerol lipase alpha. Biochem Biophys Res Comm 411:809-814.
Piomelli (2003) The molecular logic of endocannabinoid signalling. Nat Rev Neurosci 4:873-884.
Piomelli (2005) The endocannabinoid system: a drug discovery perspective. Curr Opin Investig Drugs 6:672-679.
Piomelli et al. (2000) The molecular logic of endocannabinoid signalling. Nature Rev 4:873-884.
Piomelli et al. (2003) The endocannabinoid system as a target for therapeutic drugs. Trends Pharmacol Sci 21:218-224
Potterton et al. (2003) A graphical user interface to the CCP4 program suite. Acta Cryst D59:1131-1137.
Prusakiewicz et al. (2009) Differential sensitivity and mechanism of inhibition of COX-2 oxygenation of arachidonic acid and 2-arachidonoylglycerol by ibuprofen and mefenamic acid. Biochemistry 48:7353-7355.
Rainsford (2009) Ibuprofen: pharmacology, efficacy and safety. Inflammopharmacology 17:275-342.
Remington et al. (1975) Remington's Pharmaceutical Sciences, 15th ed., Mack Pub. Co., Easton, Pa.
Richie-Jannetta et al. (2010) Structural determinants for calcium mobilization by prostaglandin E2 and prostaglandin F2a glyceryl esters in RAW 264.7 cells and H1819 cells. Prostaglandins Other Lipid Mediat 92:19-24.
Rimon et al. (2010) Coxibs interfere with the action of aspirin by binding tightly to one monomer of cyclooxygenase-1. Proc Natl Acad Sci US A 107:28-33.
Rossi et al. (2010) Preservation of striatal cannabinoid CB1 receptor function correlates with the antianxiety effects of fatty acid amide hydrolase inhibition. Mol Pharmacol 78, 260-268.
Rouzer & Marnett (2005) Glyceryl prostaglandin synthesis by resident peritoneal macrophages in response to a zymosan stimulus. J Biol Chem 280:26690-26700.
Rouzer & Marnett (2008) Non-redundant functions of cyclooxygenases:
oxygenation of endocannabinoids. J Biol Chem 283:8065-8069.
Rouzer & Marnett (2009) Cyclooxygenases: Structural and Functional Insights. J Lipid Res 2009, April Supplement, S29-S34.
Rouzer & Mamett (2011) Endocannabinoid Oxygenation by Cyclooxygenases, Lipoxygenases, and Cytochromes P450: Cross-Talk between the Eicosanoid and Endocannabinoid Signaling Pathways. Chem Rev 111:5899-5921.
Rowlinson et al. (1999) The binding of arachidonic acid in the cyclooxygenase active site of mouse prostaglandin endoperoxide synthase-2 (COX-2): a putative L-shaped binding conformation utilizing the top channel region. J Biol Chem 274:23305-23310.
Sang et al. (2006) PGE2 glycerol ester, a COX-2 oxidative metabolite of 2-arachidonoyl glycerol, modulates inhibitory synaptic transmission in mouse hippocampal neurons. J Physiol (Lond) 572:735-745.
Sang et al. (2007) COX-2 oxidative metabolite of endocannabinoid 2-AG enhances excitatory glutamatergic synaptic transmission and induces neurotoxicity. J Neurochem 102:1966-1977.
Sciolino et al. (2011) Enhancement of endocannabinoid signaling with JZL184, an inhibitor of the 2-arachidonoylglycerol hydrolyzing enzyme monoacylglycerol lipase, produces anxiolytic effects under conditions of high environmental aversiveness in rats. Pharmacol Res: Official J Italian Pharmacol Soc.
Selinsky et al. (2001) Structural analysis of NSAID binding by prostaglandin H2 synthase: time-dependent and time-independent inhibitors elicit identical enzyme conformations. Biochemistry 40:5172-5180.
Snider & Hollenberg (2010) Oxidation of the endogenous cannabinoid arachidonoyl ethanolamide by the cytochrome P450 monooxygenases: physiological and pharmacological implications. Pharmacol Rev 62:136-154.
Speight et al. (1997) Avery's Drug Treatment: A Guide to the Properties, Choice, Therapeutic Use and Economic Value of Drugs in Disease Management, 4th ed. Adis International, Auckland/Philadelphia.
Staniaszek et al. (2010) Effects of COX-2 inhibition on spinal nociception: the role of endocannabinoids. Br J Pharmacol 160:669-676.
Sumislawski et al. (2011) Reversible gating of endocannabinoid plasticity in the amygdala by chronic stress: a potential role for monoacylglycerol lipase inhibition in the prevention of stress-induced behavioral adaptation. Neuropsychopharmacol 36:2750-2761.
Svizenska et al. (2008) Cannabinoid Receptors 1 and 2 (CB1 and CB2), their Distribution, Ligands and Functional Involvement in the Nervous System Structures—A Short Review. Pharmacol Biochem Behav. 90:501-511.
Tam et al. (2006) Involvement of neuronal cannabinoid receptor CB1 in regulation of bone mass and bone remodeling. Mol Pharmacol 70:786-792.
Turini et al. (2002) Cyclooxygenase-2: A Therapeutic Target. Ann Rev Med 53:35-57.
Uddin et al. (2010) Selective visualization of cyclooxygenase-2 in inflammation and cancer by targeted fluorescent imaging agents. Cancer Res 70:3618-3627.
Uddin et al. (2011) Fluorinated COX-2 Inhibitors as Agents in PET Imaging of Inflammation and Cancer. Cancer Prev Res 4:1536-1545.
Ueda et al. (1995) Lipoxygenase-catalyzed oxygenation of arachidonylethanolamide, a cannabinoid receptor agonist. Biochim Biophys Acta 1254:127-134.
Ueda et al. (2011) Biosynthesis and degradation of the endocannabinoid 2-arachidonoylglycerol. BioFactors 37:1-7.
U.S. Patent Application Publication No. 2006/0188558.
U.S. Pat. Nos. 3,598,122; 5,016,652; 5,234,933; 5,326,902; 5,935,975; 5,973,191; 6,106,856; 6,162,459; 6,180,082; 6,207,700; 6,284,918; 6,306,890; 6,399,647; 6,495,605; 6,582,724; 6,762,182; 7,189,504; 7,314,709; 7,491,744; 7,628,975; 7,736,624; 8,143,302; 8,168,656.
Vagin & Teplyakov (2000) An approach to multi-copy search in molecular replacement. Acta Crystallogr D Biol Crystallogr 56:1622-1624.
Wang et al. (2008) Loss of Cannabinoid Receptor 1 Accelerates Intestinal Tumor Growth. Cancer Res 68:6468-6476.
Woodward et al. (2008) The pharmacology and therapeutic relevance of endocannabinoid derived cyclo-oxygenase (COX)-2 products. Pharmacol Ther 120:71-80.
Woodman et al. (2011) Chiral inversion of 2-arylpropionyl-CoA esters by human alpha-methylacyl-CoA racemase 1A (P504S)—a potential mechanism for the anti-cancer effects of ibuprofen. Chem Comm 47:7332-7334.
Wu et al. (2009) Glial precursors clear sensory neuron corpses during development via Jedi-1, an engulfment receptor. Nat Neurosci 12:1534-1541.
Xie et al. (2010) Inactivation of lipid glyceryl ester metabolism in human THP1 monocytes/macrophages by activated organophosphorus insecticides: role of carboxylesterases 1 and 2. Chem Res Toxicol 23:1890-1904.
Yang & Chen (2008) Cyclooxygenase-2 in synaptic signaling. Curr Pharm Des 14:1443-1451.
Yokoyama & Tanabe (1989) Cloning of Human Gene Encoding Prostaglandin Endoperoxide Synthase and Primary Structure of the Enzyme. Biochem Biophys Res Commun 165:888-894.
Yu et al. (1997) Synthesis of prostaglandin E2 ethanolamide from anandamide by cyclooxygenase-2. J Biol Chem 272:21181-21186.
Yu et al. (2012) Vascular COX-2 modulates blood pressure and thrombosis in mice. Science Translational Med 4:132ra154.
Yuan et al. (2006) Partnering between monomers of cyclooxygenase-2 homodimers. Proc Natl Acad Sci USA 103:6142-6147.
Yuan et al. (2009) Cyclooxygenase Allosterism, Fatty Acid-mediated Cross-talk between Monomers of Cyclooxygenase Homodimers. J Biol Chem 284:10046-10055.
Zhao et al. (2010) One-Pot Synthesis of Aminoenone via Direct Reaction of the Chloroalkyl Enone with NaN3: Rapid Access to Polycyclic Alkaloids. J Org Chem 75:5289-5295.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A pharmaceutical composition comprising, consisting essentially of, or consisting of a substrate-selective inhibitor of cyclooxygenase-2 (COX-2) and a pharmaceutically acceptable carrier or excipient, optionally wherein the pharmaceutically acceptable carrier or excipient is acceptable for use in a human.

2. The pharmaceutical composition of claim 1, wherein the substrate-selective inhibitor of COX-2 comprises a substantially pure (R)-profen or a derivative thereof, optionally wherein the derivative thereof does not stereoisomerize in vivo to an (S)-profen.

3. The pharmaceutical composition of claim 2, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

4. A method for selectively inhibiting endocannabinoid oxygenation but not arachidonic acid oxygenation, the method comprising contacting a COX-2 polypeptide with an effective amount of a substrate-selective COX-2 inhibitor.

5. The method of claim 4, wherein the substrate-selective inhibitor of COX-2 comprises a substantially pure (R)-profen or a derivative thereof, optionally wherein the derivative thereof does not stereoisomerize in vivo to an (S)-profen.

6. The method of claim 5, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

7. The method of any of claims 4-6, wherein the COX-2 polypeptide is present in a subject, optionally wherein the subject is a human.

8. A method for elevating a local endogenous cannabinoid concentration in a tissue, cell, organ, and/or structure in a subject, the method comprising contacting a COX-2 polypeptide present in the subject with an effective amount of a substrate-selective COX-2 inhibitor.

9. The method of claim 8, wherein the COX-2 polypeptide is present in the tissue, cell, organ, and/or structure in the subject.

10. The method of claim 8, wherein the substrate-selective inhibitor of COX-2 comprises a substantially pure (R)-profen or a derivative thereof, optionally wherein the derivative thereof does not stereoisomerize in vivo to an (S)-profen.

11. The method of claim 9, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

12. The method of any of claims 8-11, wherein the subject is a human.

13. A method of reducing depletion of an endogenous cannabinoid in a tissue, cell, organ, and/or structure in a subject, the method comprising contacting a COX-2 polypeptide present in the subject with an effective amount of a substrate-selective COX-2 inhibitor.

14. The method of claim 13, wherein the COX-2 polypeptide is present in the tissue, cell, organ, and/or structure in the subject and/or is present in a distant location in the subject that under normal conditions provides an endogenous cannabinoid to the tissue, cell, organ, and/or structure in the subject.

15. The method of claim 13, wherein the substrate-selective inhibitor of COX-2 comprises a substantially pure (R)-profen or a derivative thereof, optionally wherein the derivative thereof does not stereoisomerize in vivo to an (S)-profen.

16. The method of claim 15, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

17. The method of any of claims 13-16, wherein the subject is a human.

18. The method of any of claims 13-16, wherein the COX-2 polypeptide is present in a region of inflammation in the subject.

19. A method for inducing analgesia in a subject, the method comprising contacting a COX-2 polypeptide present in the subject with an effective amount of a substrate-selective COX-2 inhibitor.

20. The method of claim 19, wherein the substrate-selective inhibitor of COX-2 comprises a substantially pure (R)-profen or a derivative thereof, optionally wherein the derivative thereof does not stereoisomerize in vivo to an (S)-profen.

21. The method of claim 20, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of —Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

22. The method of any of claims 19-21, wherein the subject is a human.

23. The method of any of claims 19-21, wherein the COX-2 polypeptide is present in a region of inflammation in the subject.

24. A method of providing an anxiolytic therapy, an antidepressant therapy, or both to a subject, the method comprising contacting a COX-2 polypeptide present in the subject with an effective amount of a substrate-selective COX-2 inhibitor.

25. The method of claim 24, wherein the substrate-selective inhibitor of COX-2 comprises a substantially pure (R)-profen or a derivative thereof, optionally wherein the derivative thereof does not stereoisomerize in vivo to an (S)-profen.

26. The method of claim 25, wherein the (R)-profen or a derivative thereof is selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, an (R)-enantiomer of Compound 6e, acetaminophen (APAP), 4-aminophenol, and N-(4-hydroxyphenyl)arachidonoylamide (AM404), and combinations thereof.

27. The method of any of claims 24-26, wherein the subject is a human.

28. The method of any of claims 24-26, wherein the COX-2 polypeptide is present in a region of inflammation in the subject.

29. A compound selected from the group consisting of Compound A, Compounds 3a-3e, Compounds 4a-4e, Compounds 5a-5e, Compounds 7a-7e, an (R)-enantiomer of Compound 6a, an (R)-enantiomer of Compound 6b, an (R)-enantiomer of Compound 6c, an (R)-enantiomer of Compound 6d, and an (R)-enantiomer of Compound 6e.
